(19) United States
(12) Patent Application Publication
(10) Pub. No.: US 2008/0153783 A1
(43) Pub. Date: Jun. 26, 2008

(54) Pyrimidyl Phosphonate Antiviral Compounds and Methods of Use

(75) Inventors: Haolun Jin, Foster City, CA (US); Choung U. Kim, San Carlos, CA (US)

Correspondence Address: GILEAD SCIENCES INC, 333 LAKESIDE DR, FOSTER CITY, CA, 94404, US

(73) Assignee: GILEAD SCIENCES, INC., Foster City, CA (US)

(21) Appl. No.: 10/585,504

(22) PCT Filed: Nov. 1, 2005
(86) PCT No.: PCT/US2005/000815

§ 371 Date: Feb. 14, 2008

Related U.S. Application Data
(60) Provisional application No. 60/536,010, filed on Jan. 12, 2004.

(51) Int. Cl. A61K 31/675; C07F 9/54; A61P 31/12

(52) U.S. Cl. 514/86; 544/243

(57) ABSTRACT

Pyrimidine I and pyrimidinone II phosphonate compounds and methods for viral inhibition are disclosed. The compounds include at least one phosphonate group covalently attached at any site.

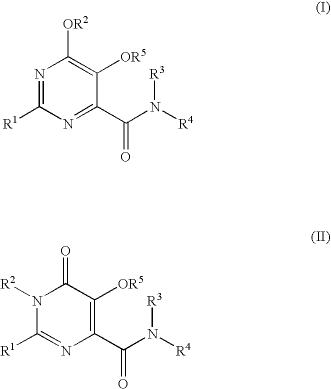

### FIELD OF THE INVENTION

The invention relates generally to compounds with antiviral activity and more specifically with HIV-integrase inhibitory properties.

### BACKGROUND OF THE INVENTION

Human immunodeficiency virus (HIV) infection and related diseases are a major public health problem worldwide. A virally encoded integrase protein mediates specific incorporation and integration of viral DNA into the host genome. Integration is essential for viral replication. Accordingly, inhibition of HIV integrase is an important therapeutic pursuit for treatment of HIV infection of the related diseases.

Human immunodeficiency virus type 1 (HIV-1) encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, etal N. Eng. J. Med. (1998) 338:853-860, Richman, D. D. Nature (2001) 410:995-1001). There is a need for new agents directed against alternate sites in the viral life cycle. Integrase has emerged as an attractive target, because it is necessary for stable infection and homologous enzymes are lacking in the human host (LaFemina, etal J. Virol. (1992) 66:7414-7419). The function of integrase is to catalyze integration of proviral DNA, resulting from the reverse transcription of viral RNA, into the host genome, by a stepwise fashion of endonucleolytic processing of proviral DNA within a cytoplasmic preintegration complex (termed 3′-processing or “3′-P”) with specific DNA sequences at the end of the HIV-1 long terminal repeat (LTR) regions, followed by translocation of the complex into the nuclear compartment where integration of 3′-processed proviral DNA into host DNA occurs in a “strand transfer” (ST) reaction (Hazuda, etal Science (2000) 287:646-650; Katzman, etal Adv. Virus Res. (1999) 52:371-395; Asante-Applah, etal Adv. Virus Res. (1999) 52:351-369). Although numerous agents potently inhibit 3′-P and ST in extracellular assays that employ recombinant integrase and viral long-terminal-repeat oligonucleotide sequences, often such inhibitors lack inhibitory potency when assayed using fully assembled preintegration complexes or fail to show antiviral effects against HIV-infected cells (Pommier, etal Adv. Virus Res. (1999) 52:427-458; Farnet, etal Proc. Natl. Acad. Sci. U.S.A. (1996) 93:9742-9747; Pommier, etal Antiviral Res. (2000) 47:139-148.

Certain HIV integrase inhibitors have been disclosed which block integration in extracellular assays and exhibit good antiviral effects against HIV-infected cells (Anthony, etal WO 02/30426; Anthony, etal WO 02/30930; Anthony, etal WO 02/30931; WO 02/055079; Zhuang, etal WO 02/36734; U.S. Pat. No. 6,395,743; U.S. Pat. No. 6,245,806; U.S. Pat. No. 6,271,402; Fujishita, etal WO 00/039086; Uenaka etal WO 00/075122; Selnick, etal WO 99/62513; Young, etal WO 99/62520; Payne, etal WO 01/00578; Jing, etal Biochemistry (2002) 41:5397-5403; Pais, etal Jour. Med. Chem. (2002) 45:3184-94; Goldgur, etal Proc. Natl. Acad. Sci. U.S.A. (1999) 96:13040-13043; Espeseth, etal Proc. Natl. Acad. Sci. U.S.A. (2000) 97:11244-11249).

HIV integrase inhibitory compounds with improved antiviral and pharmacokinetic properties are desirable, including enhanced activity against development of HIV resistance, improved oral bioavailability, greater potency and extended effective half-life in vivo (Nair, V. “HIV integrase as a target for antiviral chemotherapy” Reviews in Medical Virology (2002) 12(3):179-193; Young (2001) Current Opinion in Drug Discovery & Development, Vol. 4, No. 4, 402-410; Neamati (2002) Expert. Opin. Ther. Patents Vol. 12, No. 5, 709-724). Three-dimensional quantitative structure-activity relationship studies and docking simulations (Buolamwini, etal Jour. Med. Chem. (2002) 45:841-852) of conformationally-restrained cinnamoyl-type integrase inhibitors (Artico, etal Jour. Med. Chem. (1998) 41:3948-3960) have shown a large contribution of hydrogen-bonding interactions to the inhibitory activity differences among the compounds. Conformationally-constrained hydrogen-bonding functionality such as hydroxyl was correlated with inhibitory activity. Compounds with binding functionality in a pre-organized configuration may possess optimized inhibitory properties against HIV integrase. The prior art does not show or suggest compounds with integrase binding functionality in a pre-organized conformation or molecular structure. In addition to therapeutic uses, the value of compounds in diagnostic assays for HIV, for use in the preparation of polymers and for use as surfactants, and in other industrial utilities will be readily apparent to those skilled in the art.

Dihydroxypyrimidine carboxamide (WO 03/03507.6A1) and N-substituted hydroxypyrimidinone carboxamide (WO 03/035077A1) compounds have been reported to have HIV integrase inhibitory properties.

Improving the delivery of drugs and other agents to target cells and tissues has been the focus of considerable research for many years. Though many attempts have been made to develop effective methods for importing biologically active molecules into cells, both in vivo and in vitro, none has proved to be entirely satisfactory. Optimizing the association of the inhibitory drug with its intracellular target, while minimizing intercellular redistribution of the drug, e.g. to neighboring cells, is often difficult or inefficient.

Most agents currently administered to a patient parenterally are not targeted, resulting in systemic delivery of the agent to cells and tissues of the body where it is unnecessary, and often undesirable. This may result in adverse drug side effects, and often limits the dose of a drug (e.g., cytotoxic agents and other anti-cancer or anti-viral drugs) that can be administered. By comparison, although oral administration of drugs is generally recognized as a convenient and economical method of administration, oral administration can result in either (a) uptake of the drug through the cellular and tissue barriers, e.g. blood/brain, epithelial, cell membrane, resulting in undesirable systemic distribution, or (b) temporary residence of the drug within the gastrointestinal tract. Accordingly, a major goal has been to develop methods for specifically targeting agents to cells and tissues. Benefits of such treatment includes avoiding the general physiological effects of inappropriate delivery of such agents to other cells and tissues, such as uninfected cells. Intracellular targeting may be achieved by methods and compositions which allow accumulation or retention of biologically active agents inside cells.

### SUMMARY OF THE INVENTION

The present invention provides compositions and methods for inhibition of viruses, including HIV. Compositions and methods of the present invention inhibit HIV-integrase.

In one aspect, the invention includes 4,5-dihydroxypyrimidine, 6-carboxamide phosphonate compounds having Formula I:

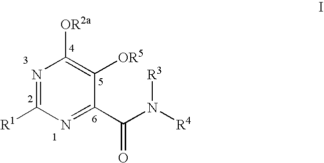

In another aspect, the invention includes 3-N-substituted, 5-hydroxypyrimidinone, 6-carboxamide phosphonate compounds having Formula II:

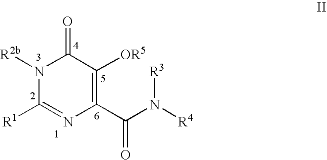

The invention includes pharmaceutically acceptable salts of Formulas I and II, and enol and tautomeric resonance isomers thereof.

Formula I and II compounds are substituted with one or more covalently attached phosphonate groups. The compounds of the invention include at least one phosphonate group covalently attached at any site, i.e. R¹, R^{2a}, R^{2b}, R³, R⁴ or R⁵.

The invention also includes a pharmaceutical composition comprising an effective amount of a compound selected from Formula I or Formula II, or a pharmaceutically acceptable salt thereof, in combination with a pharmaceutically acceptable diluent or carrier.

This invention also includes a method of increasing cellular accumulation and retention of drug compounds, thus improving their therapeutic and diagnostic value.

The invention also includes a method of inhibiting HIV, comprising administering to a mammal infected with HIV (HIV positive) an amount of a compound of Formula I or Formula II, effective to inhibit the growth of said HIV infected cells.

The invention also includes a compound selected from Formula I or Formula II for use in medical therapy (preferably for use in treating cancer, e.g. solid tumors), as well as the use of a compound of Formula I or Formula II for the manufacture of a medicament useful for the treatment of cancer, e.g. solid tumors.

The invention also includes processes and novel intermediates disclosed herein which are useful for preparing compounds of the invention. Some of the compounds of Formula I or Formula II are useful to prepare other compounds of Formula I or Formula II.

In another aspect of the invention, the activity of HIV integrase is inhibited by a method comprising the step of treating a sample suspected of containing HIV virus with a compound or composition of the invention.

Another aspect of the invention provides a method for inhibiting the activity of HIV integrase comprising the step of contacting a sample suspected of containing HIV virus with the composition embodiments of the invention.

In other aspects, novel methods for the synthesis, analysis, separation, isolation, crystallization, purification, characterization, and testing of the compounds of this invention are provided.

### DETAILED DESCRIPTION OF EXEMPLARY EMBODIMENTS

Reference will now be made in detail to certain embodiments of the invention, examples of which are illustrated in the accompanying descriptions, structure and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims.

### Definitions

Unless stated otherwise, the following terms and phrases as used herein are intended to have the following meanings:

The terms “phosphonate” and “phosphonate group” mean a functional group or moiety within a molecule that comprises at least one phosphorus-carbon bond, and at least one phosphorus-oxygen double bond. The phosphorus atom is further substituted with oxygen, sulfur, and nitrogen substituents. These substituents may be part of a prodrug moiety. As defined herein, “phosphonate” and “phosphonate group” include phosphonic acid, phosphonic monoester, phosphonic diester, diphosphophosphonate, phosphonamidate, phosphondiamidate, and phosphonthioate functional groups; and the group A³.

The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound.

“Pharmaceutically acceptable prodrug” refers to a compound that is metabolized in the host, for example hydrolyzed or oxidized, by either enzymatic action or by general acid or base solvolysis, to form an active ingredient. Typical examples of prodrugs of the compounds of the invention have biologically labile protecting groups on a functional moiety of the compound. Prodrugs include compounds that can be oxidized, reduced, aminated, deaminated, esterified, deesterified, alkylated, dealkylated, acylated, deacylated, phosphorylated, dephosphorylated, photolyzed, hydrolyzed, or other functional group change or conversion involving forming or breaking chemical bonds on the prodrug.

“Prodrug moiety” means a labile functional group which separates from the active inhibitory compound during metabolism, systemically, inside a cell, by hydrolysis, enzymatic cleavage, or by some other process (Bundgaard, Hans, “Design and Application of Prodrugs” in Textbook of Drug Design and Development (1991), P. Krogsgaard-Larsen and H. Bundgaard, Eds. Harwood Academic Publishers, pp. 113-191). Enzymes which are capable of an enzymatic activation mechanism with the phosphonate prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy. A “prodrug” is thus a covalently modified analog of a therapeutically-active compound. A prodrug moiety may include an active metabolite or drug itself.

Exemplary prodrug moieties include the hydrolytically sensitive or labile acyloxymethyl esters —CH₂C(═O)R⁹ and acyloxymethyl carbonates —CH₂C(═O)OR⁹ where R⁹ is C₁-C₆ alkyl, C₁-C₆ substituted alkyl, C₆-C₂₀ aryl or C₆-C₂₀ substituted aryl. The acyloxyalkyl ester was first used as a prodrug strategy for carboxylic acids and then applied to phosphates and phosphonates by Farquhar etal (1983) J. Pharm. Sci. 72: 324; also U.S. Pat. Nos. 4,816,570, 4,968,788, 5,663,159 and 5792756. In certain compounds of the invention, a prodrug moiety is part of a phosphonate group. Subsequently, the acyloxyalkyl ester was used to deliver phosphonic acids across cell membranes and to enhance oral bioavailability. A close variant of the acyloxyallyl ester, the alkoxycarbonyloxyallcyl ester (carbonate), may also enhance oral bioavailability as a prodrug moiety in the compounds of the combinations of the invention. An exemplary acyloxymethyl ester is pivaloyloxymethoxy, (POM)-CH₂C(═O)C(CH₃)₃. Exemplary acyloxymethyl carbonate prodrug moieties are pivaloyloxymethylcarbonate (POC) —CH₂C(═O)OC(CH₃)₃ and —CH₂C(═O)OCH(CH₃)₂.

The phosphonate group may be a phosphonate prodrug moiety. The prodrug moiety may be sensitive to hydrolysis, such as, but not limited to a pivaloyloxymethyl carbonate (POC) or POM group. Alternatively, the prodrug moiety may be sensitive to enzymatic potentiated cleavage, such as a lactate ester or a phosphonamidate-ester group.

Aryl esters of phosphorus groups, especially phenyl esters, are reported to enhance oral bioavailability (DeLambert etal (1994) J. Med. Chem. 37: 498). Phenyl esters containing a carboxylic ester ortho to the phosphate have also been described (Khamnei and Torrence, (1996) J. Med. Chem. 39:4109-4115). Benzyl esters are reported to generate the parent phosphonic acid. In some cases, substituents at the ortho- or para-position may accelerate the hydrolysis. Benzyl analogs with an acylated phenol or an allylated phenol may generate the phenolic compound through the action of enzymes, e.g. esterases, oxidases, etc., which in turn undergoes cleavage at the benzylic C—O bond to generate the phosphoric acid and the quinone methide intermediate. Examples of this class of prodrugs are described by Mitchell etal (1992) J. Chem. Soc. Perkin Trans. I 2345; Brook etal WO 91/19721. Still other benzylic prodrugs have been described containing a carboxylic ester-containing group attached to the benzylic methylene (Glazier etal WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesterification or reduction of the disulfide generates the free thio intermediate which subsequently breaks down to the phosphoric acid and episulfide (Puech etal (1993) Antiviral Res., 22: 155-174; Benzaria etal (1996) J. Med. Chem. 39: 4958). Cyclic phosphonate esters have also been described as prodrugs of phosphorus-containing compounds (Erion etal, U.S. Pat. No. 6,312,662).

“Protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: Protective Groups in Organic Chemistry Theodora W. Greene (John Wiley & Sons, Inc., New York, 1991. Protecting groups are often utilized to mask the reactivity of certain functional groups, to assist in the efficiency of desired chemical reactions, e.g. making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g. alcohols, be physiologically acceptable, although in general it is more desirable if the products are pharmacologically innocuous.

Any reference to any of the compounds of the invention also includes a reference to a physiologically acceptable salt thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of an hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (1-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH—CH₂), cyclopentenyl (—C₅H₇), and 5-hexenyl (—CH₂ CH₂CH₂CH₂CH═CH₂).

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH),

The terms “alkylene” and “alkyldiyl” each refer to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical allylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene, i.e. double carbon-carbon bond moiety. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne, i.e. triple carbon-carbon bond moiety. Typical alkynylene radicals include, but are not limited to: acetylene (—C≡C—), propargyl (—CH₂C≡C—), and 4-pentynyl (—CH₂CH₂CH₂C≡CH—).

“Aryl” means a monovalent aromatic hydrocarbon radical of 6-20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like.

“Arylene” means a divalent aromatic hydrocarbon radical, i.e. aryldiyl, of 6-20 carbon atoms derived by the removal of two hydrogen atoms from carbon or non-carbon atoms of a parent aromatic ring system. Typical arylene groups include, but are not limited to, radicals derived from benzene, such as 1,2 phenydiyl, 1,3 phenyldiyl, and 1,4 phenyldiyl; as well as allyl-substituted benzene, such as toluene which provides

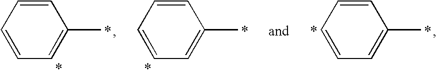

where the (“*”) indicates the points of attachment.

“Heterocycle” means a monovalent aromatic radical of one or more carbon atoms and one or more atoms selected from N, O, S, or P, derived by the removal of one hydrogen atom from a single atom of a parent aromatic ring system. Heterocyclic groups may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S). Heterocyclic bicycles have 7 to 10 ring atoms (6 to 9 carbon atoms and 1 to 2 heteroatoms selected from N, O, and S) arranged as a bicyclo [4,5], [5,5], [5,6], or [6,6] system; or 9 to 10 ring atoms (8 to 9 carbon atoms and 1 to 2 hetero atoms selected from N and S) arranged as a bicyclo [5,6] or [6,6] system. The heterocyclic group may be bonded to the drug scaffold through a carbon, nitrogen, sulfur, phosphorus or other atom by a stable covalent bond.

Heterocycle groups include, for example: pyridyl, dihydropyridyl isomers, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

Substituted substituents such as “substituted alkyl”, “substituted aryl”, “substituted heterocycle” and “substituted arylalkyl” mean alkyl, aryl, and arylallyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(—O)R, —C(═O)NRR —S(═O)₂O⁻, —S(—O)₂OH, —S(—O)₂R, —OS(═O)₂OR, —S(═O)₂NR, —S(═O)R, —OP(═O)O₂RR, —P(═O)O₂RR —P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen: F, Cl, Br, or I; and each R is independently —H, alkyl, aryl, heterocycle, protecting group or prodrug moiety. Alkylene, alkenylene, and alkynylene groups may also be similarly substituted.

“Heterocycle” means a saturated, unsaturated or aromatic ring system including at least one N, O, S, or P. Heterocycle thus include heteroaryl groups. Heterocycle as used herein includes by way of example and not limitation these heterocycles described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

One embodiment of the bis-tetrahydrofuranyl group is:

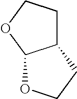

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated, unsaturated or aromatic ring system having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl. Carbocycle thus includes some aryl groups.

“Linker” or “link” means a chemical moiety comprising a covalent bond or a chain of atoms that covalently attaches a phosphonate group to a drug, or between the Formula I scaffold and substituents. Linkers include L interposed between Ar and the nitrogen of Formula I compounds. Linkers may also be interposed between a phosphorus containing A³ group and the R¹, R², R³, R⁴, R⁵, R⁶ or R⁷ positions of Formula I. Linkers include, but are not limited to moieties such as O, S, NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, C₆-C₂₀ arylene, C₆-C₂₀ substituted arylene, C(═O)NH, C(═O), S(═O)₂, C(═O)NH(CH₂)ₙ, and (CH₂CH₂O)ₙ, where n may be 1, 2, 3, 4, 5, or 6. Linkers also include repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

### Pyrimidine and Pyrimidinone Phosphonate Compounds

Novel phosphonate compounds with inhibitory activity against HIV integrase are described, as embodied in Formula I pyrimidines and Formula II pyrimidinones, including any pharmaceutically acceptable salts thereof. Formula I pyrimidine and Formula II pyrimidinone compounds each have at least one phosphonate group.

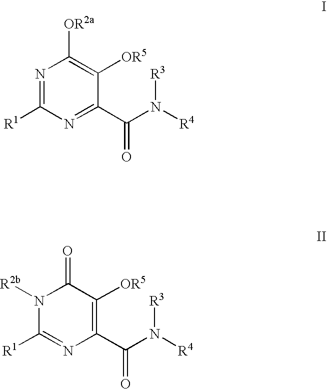

Formula I and II compounds include all pharmaceutically acceptable salts thereof. Formula I and II compounds also include all enol, tautomeric, and resonance isomers, enantiomers, diastereomers, and racemic mixtures thereof. Formula I and II compounds are related as regioisomers, constrained to their particular isomeric forms by their covalent substituents; R¹, R^{2a}, R^{2b}, R³, R⁴, and R⁵.

R¹ is selected from H, F, Cl, Br, I, OH, OR, amino (—NH₂), ammonium (—NH₃⁺), alkylamino (—NHR), dialkylamino (—NR₂), trialkylammonium (—NR₃⁺), carboxyl (—CO₂H), sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), allylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₈ alkyl, C₁-C₁₈ substituted allyl, C₂-C₁₈ alkenyl, C₂-C₁₈ substituted alkenyl, C₂-C₁₈ alkynyl, C₂-C₁₈ substituted alkyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, L-A³, and a prodrug moiety.

R^{2a} and R⁵ are each independently selected from H, carboxyl (—CO₂H), sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ alkyl, C₁-C₁₈ substituted allyl, C₂-C₁₈ alkenyl, C₂-C₁₈ substituted alkenyl, C₂-C₁₈ alkynyl, C₂-C₁₈ substituted alkynyl, C₁₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, L-A³, and a prodrug moiety.

R^{2b}, R³, and R⁴ are each independently selected from H, OH, OR, amino (—NH₂), ammonium (—NH₃⁺), alkylamino (—NHR), dialkylamino (—NR₂), trialkylammonium (—NR₃⁺), carboxyl (—CO₂H), sulfate, sulfamate, sulfonate, 5-7 membered ring sultam, 4 dialkylaminopyridinium, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, L-A³, and a prodrug moiety.

R is independently selected from H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, C₂-C₂₀ substituted heterocycle, phosphonate, phosphate, polyethyleneoxy, a protecting group, and a prodrug moiety.

Substituted alkyl, substituted alkenyl, substituted alkynyl, substituted aryl, and substituted heterocycle are independently substituted with one or more substituents selected from F, Cl, Br, I, OH, amino (—NH₂), ammonium (—NH₃⁺), alkylamino (—NHR), dialkylamino (—NR₂), trialkylammonium (—NR₃⁺), C₁-C₈ alkyl, C₁-C₈ alkylhalide, carboxylate, thiol (—SH), sulfate (—OSO₃R), sulfamate, sulfonate (—SO₃R), 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), alkylsulfoxide (—SOR), ester (—C(═O)OR), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ alkyl, C₁-C₈ substituted alkyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, and a prodrug moiety.

Embodiments of R¹, R^{2a}, R^{2b}, R³, R⁴, and R⁵ include —C(═S)NR₂, —C(═O)OR, —C(═O)NR₂, —C(═O)NRNR₂, —C(═O)R, —SO₂NR₂, —NRSO₂R, —NRC(═S)NR₂, —SR, —S(O)R, —SO₂R, —SO₂R, —P(═O)(OR)₂, —P(═O)(OR)(NR₂), —P(═O)(NR₂)₂, —P(═S)(OR)₂, —P(═S)(OR)(NR₂), —P(═S)(NR₂)₂, and including prodrug substituted forms thereof.

Embodiments of R¹, R^{2a}, R^{2b}, R³, R⁴, and R⁵ may also individually or in combination form a ring, e.g. 4-7 membered ring lactam, carbonate, or sultam, or piperazinyl sulfamate:

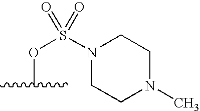

Embodiments of R¹ also include —OC(═S)NR₂, —OC(═O)OR, —OC(═O)NR₂, —OC(═O)NRNR₂, —OC(═O)R, —OP(═O)(OR)₂, —OP(═O)(OR)(NR₂), —OP(═O)(NR₂)₂, —OP(═S)(OR)₂, —OP(═S)(OR)(NR₂), —OP(═S)(NR₂)₂, and including prodrug substituted forms thereof.

A linker may be interposed between positions R¹, R², R³, R⁴, or R⁵ and substituent A³, as exemplified in some structures herein as “L-A³”. The linker L may be O, S, NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, C(═O)NH, C(═O), S(═O)₂, C(═O)NH(CH₂)ₙ, and (CH₂CH₂O)ₙ, where n may be 1, 2, 3, 4, 5, or 6. Linkers may also be repeating units of allyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide. For example, the linker may comprise propargyl, urea, or alkoxy groups.

A³ has the structure:

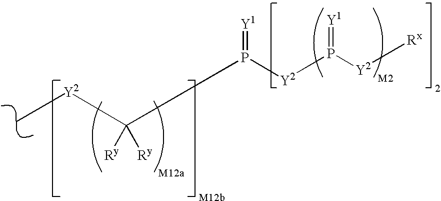

where:

Y¹ is independently O, S, NR^{x}, N(O)(R^{x}), N(OR^{x}), N(O)(OR^{x}), or N(N(R^{x})₂);

Y² is independently a bond, O, NR^{x}, N(O)(R^{x}), N(OR^{x}), N(O)(OR^{x}), N(N(R^{x})₂), —S(O)— (sulfoxide), —S(O)₂— (sulfone), —S-(sulfide), or —S—S-(disulfide);

M2 is 0, 1 or 2;

M12a is 1, 2, 3, 4, 5, 6, 7, 8, 9, 10, 11, or 12; and

M12b is 0, 1, 2, 3, 4, 5, 6, 7, 8, 9, 10, 11, or 12.

R^{y} is independently H, C₁-C₁₈ alkyl, C₁-C₁₈ substituted alkyl, C₆-C₂₀ aryl, C₆-C₂₀ substituted aryl, or a protecting group, or where taken together at a carbon atom, two vicinal R^{y} groups form a carbocycle or a heterocycle. Alternatively, taken together at a carbon atom, two vicinal R^{y} groups form a ring, i.e. a spiro carbon. The ring may be all carbon atoms, for example, cyclopropyl, cyclobutyl, cyclopentyl, or cyclohexyl, or alternatively, the ring may contain one or more heteroatoms, for example, piperazinyl, piperidinyl, pyranyl, or tetrahydrofuryl.

R^{x} is independently H, C₁-C₁₈ alkyl, C₁-C₁₈ substituted alkyl, C₆-C₂₀ aryl, C₆-C₂₀ substituted aryl, or a protecting group, or the formula:

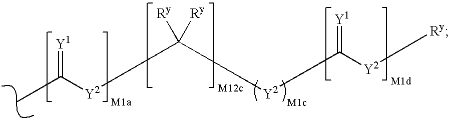

where M1a, M1c, and M1d are independently 0 or 1, and M12c is 0, 1, 2, 3, 4, 5, 6, 7, 8, 9, 10, 11 or 12.

At least one of R, R¹, R^{2a}, R^{2b}, R³, R⁴, and R⁵ in each Formula I and Formula II compound comprises a phosphonate group.

Exemplary embodiments of C₆-C₂₀ substituted aryl groups include halo-substituted phenyl such as 4-fluorophenyl, 4-chlorophenyl, 3,5-dichlorophenyl, and 3,5-difluorophenyl.

Ar groups include:

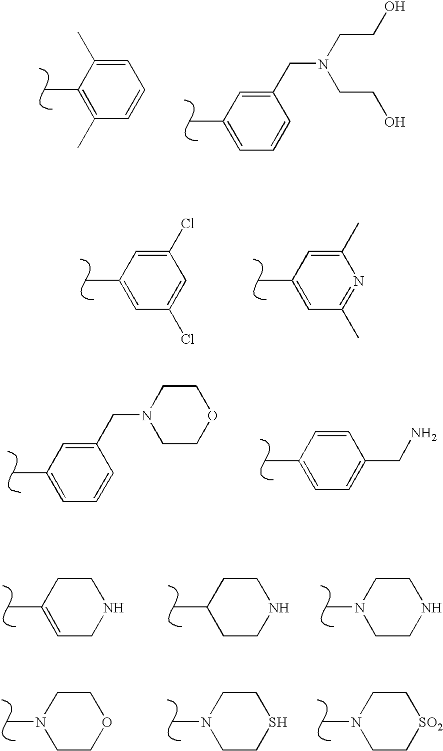

where a wavy line [symbol] , in any orientation, indicates the covalent attachment site of the other structural moieties of the compound.

Examples of substituted phenyl groups include:

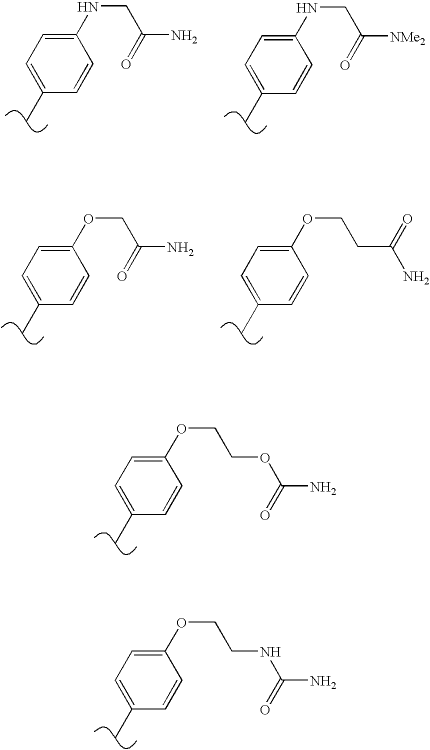

A compound of the invention includes one or more phosphonate group or phosphonate prodrug moiety. At least one of R¹, R^{2a}, R^{2b}, R³, R⁴, and R⁵ comprises a phosphonate group. The phosphonate group may be a prodrug moiety. The phosphonate group may be directly attached to a carbon, nitrogen or oxygen atom of Formula I or Formula II. Alternatively, and by example, R¹, R^{2a}, R^{2b}, R³, R⁴, and R⁵ may comprise the structure A³.

Embodiments of A³ include where M2 is 0, such as:

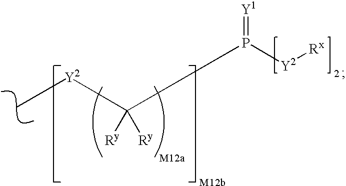

and where M12b is 1, Y¹ is oxygen, and Y^{2b} is independently oxygen (O) or nitrogen (N(R^{x})) such as:

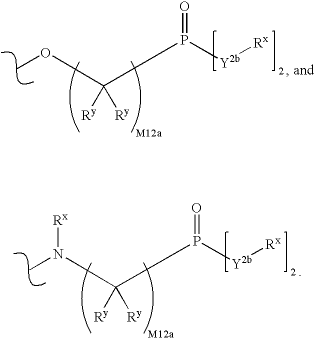

Embodiments of A³ include where Y¹ is O, resulting in the structure:

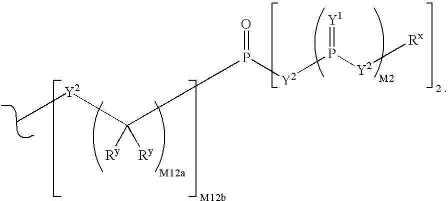

Embodiments of A³ include Where Y² is O, and M2 is 0, resulting in the structure:

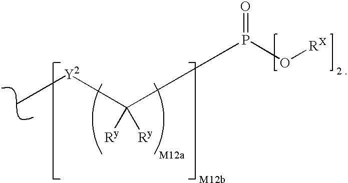

Embodiments of A³ include where R^{y} is H, and M 12a is 2, resulting in the structure:

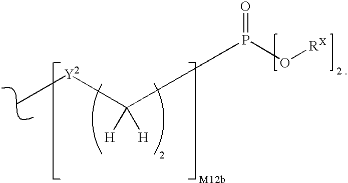

Embodiments of A³ include where Y² is —N(CH₃)—, and M12b is 1, resulting in the structure:

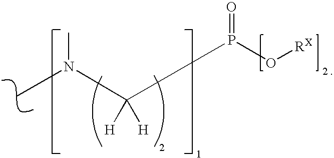

Embodiments of A³ include the following structure;

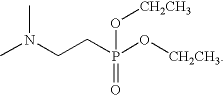

Embodiments of A³ include the following structure,

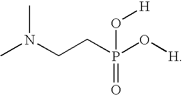

An embodiment of A³ includes:

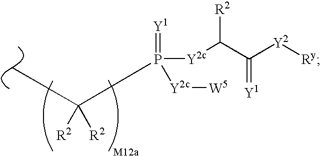

where W⁵ is a carbocycle such as phenyl or substituted phenyl, and Y²c is independently O, N(R^{y}) or S. For example, R¹ may be H and n may be 1.

W⁵ also includes, but is not limited to, aryl and heterocycle groups such as:

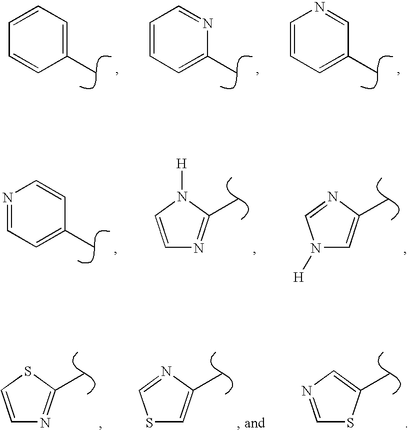

Another embodiment of A³ includes:

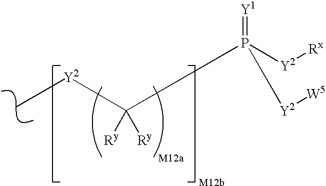

Such embodiments include:

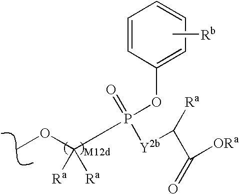

where Y^{2b} is O or N(R^{x}); M12d is 1, 2, 3, 4, 5, 6, 7 or 8; R^{a} is H or C₁-C₆ alkyl; and the phenyl carbocycle is substituted with 0 to 3 R^{b} groups where R^{b} is C₁-C₆ alkyl or substituted alkyl. Such embodiments of A³ include phenyl phosphonamidate amino acid, e.g. alanate esters and phenyl phosphonate-lactate esters:

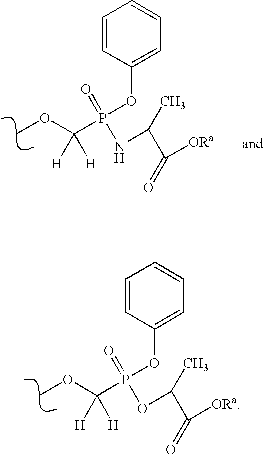

Embodiments of R^{x} include esters, carbamates, carbonates, thioesters, amides, thioamides, and urea groups:

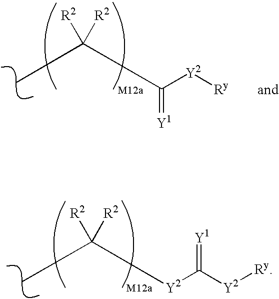

Exemplary structures within Formula I include Ia, Ib, Ic, Id:

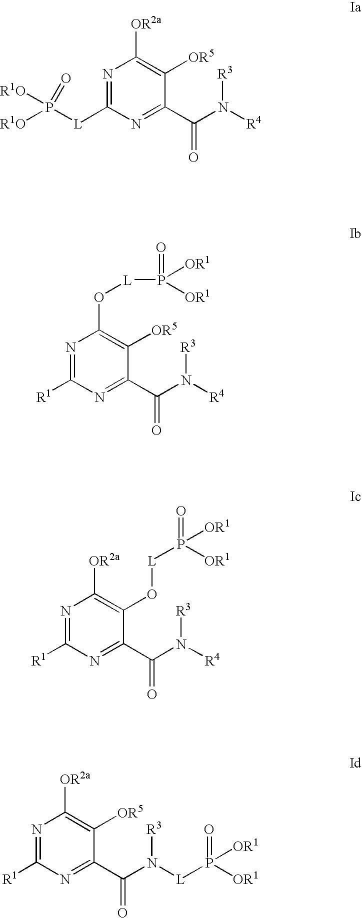

Exemplary structures within Formula II include IIa, IIb, IIc, IId:

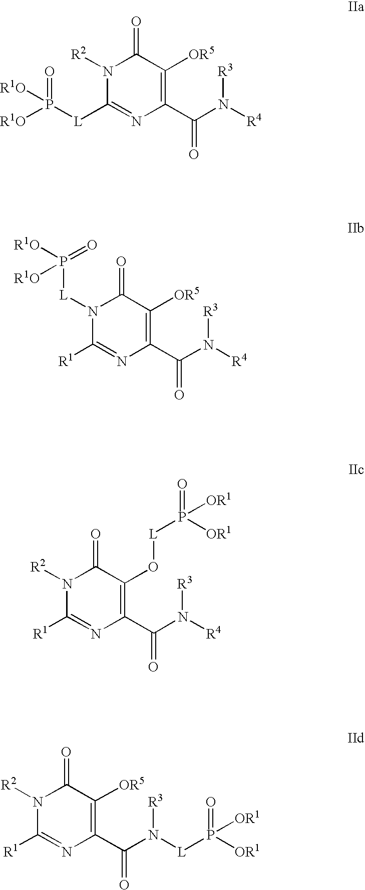

The compounds of the invention include one or more prodrug moieties located as a covalently-attached substituent at any location of Formula I or Formula II, e.g. R¹, R^{2a}, R^{2b}, R³, R⁴, or R⁵. One substituent which may be modified as a prodrug moiety is a phosphonate, phosphate, phosphinate or other phosphorus functionality (Oliyai etal Pharmaceutical Res. (1999) 16:1687-1693; Krise, J. and Stella, V. Adv. Drug Del. Reviews (1996) 19:287-310; Bischofberger etal, U.S. Pat. No. 5,798,340). Prodrug moieties of phosphorus functionality serve to mask anionic charges and decrease polarity. The phosphonate prodrug moiety may be an ester (Oliyai, etal Intl. Jour. Pharmaceutics (1999) 179:257-265), e.g. POC and POM (pivaloyloxymethyl, Yuan, etal Pharmaceutical Res. (2000) 17:1098-1103), or amidate which separates from the integrase inhibitor compound in vivo or by exposure in vitro to biological conditions, e.g. cells, tissue isolates. The separation may be mediated by general hydrolytic conditions, oxidation, enzymatic action or a combination of steps.

Compounds of the invention bearing one or more prodrug moieties may increase or optimize the bioavailability of the compounds as therapeutic agents. For example, bioavailability after oral administration may be preferred and depend on resistance to metabolic degradation in the gastrointestinal tract or circulatory system, and eventual uptake inside cells. Prodrug moieties are considered to confer said resistance by slowing certain hydrolytic or enzymatic metabolic processes. Lipophilic prodrug moieties may also increase active or passive transport of the compounds of the invention across cellular membranes (Darby, G. Antiviral Chem. & Chemotherapy (1995) Supp. 1, 6:54-63).

In one aspect, the compounds of the invention include an active form for inhibition of nuclear integration of reverse-transcribed HIV DNA.

Exemplary embodiments of the invention includes phosphonamidate and phosphoramidate (collectively “amidate”) prodrug compounds. General formulas for phosphonamidate and phosphoramidate prodrug moieties include:

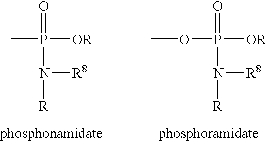

The phosphorus atom of the phosphonamidate group is bonded to a carbon atom. The nitrogen substituent R⁸ may include an ester, an amide, or a carbamate functional group. For example, R⁸ may be —CR₂C(═O)OR′ where R′ is H, C₁-C₆ alkyl, C₁-C₆ substituted alkyl, C₆-C₂₀ aryl, C₆-C₂₀ substituted aryl, C₂-C₂₀ heterocycle, or C₂-C₂₀ substituted heterocycle. The nitrogen atom may comprise an amino acid residue within the prodrug moiety, such as a glycine, alanine, or valine ester (e.g. valacyclovir, see: Beauchamp, etal Antiviral Chem. Chemotherapy (1992) 3:157-164), such as the general structure:

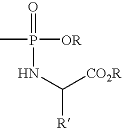

where R′ is the amino acid side-chain, e.g. H, CH₃, CH(CH₃)₂, etc.

An exemplary embodiment of a phosphonamidate prodrug moiety is:

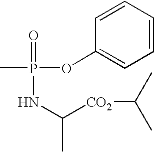

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The compounds of this invention optionally comprise salts of the compounds herein, especially pharmaceutically acceptable non-toxic salts containing, for example, Na⁺, Li⁺, K⁺, Ca⁺² and Mg⁺². Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exists in a variety of different forms, the invention is intended to encompass not only forms of the compounds that are in association with counter ions (e.g., dry salts), but also forms that are not in association with counter ions (e.g., aqueous or organic solutions).

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids, e.g., HCl, HBr, H₂SO₄, H₃PO₄ or organic sulfonic acids, to basic centers, typically amines, or to acidic groups. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention can also exist as tautomeric, resonance isomers in certain cases. Typically, the structures shown herein exemplify only one tautomeric or resonance form of the compounds. For example, hydrazine, oxime, hydrazone groups may be shown in either the syn or anti configurations. The corresponding alternative configuration is contemplated as well. All possible tautomeric and resonance forms are within the scope of the invention.

One enantiomer of a compound of the invention can be separated substantially free of its opposing enantiomer by a method such as formation of diastereomers using optically active resolving agents (Stereochemistry of Carbon Compounds, (1962) by E. L. Eliel, McGraw Hill; Lochmuller, C. H., (1975) J. Chromatogr., 113:(3) 283-302). Separation of diastereomers formed from the racemic mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers. Alternatively, enantiomers can be separated directly under chiral conditions, method (3).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111).

By method (3), a racemic mixture of two asymmetric enantiomers can be separated by chromatography using a chiral stationary phase (Chiral Liquid Chromatography (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

Enantiomers can be distinguished by methods used to distinguish other chiral molecules with asymmetric carbon atoms, such as optical rotation and circular dichroism.

### Synthesis of Pyrimidine and Pyrimidinone Phosphonate Compounds

The compounds of the invention may be prepared by a variety of synthetic routes and methods known to those skilled in the art. The invention also relates to methods of making the compounds of the invention. The compounds are prepared by any of the applicable techniques of organic synthesis. Many such techniques are well known in the art. However, many of the known techniques are elaborated in: Compendium of Organic Synthetic Methods, John Wiley & Sons, New York, Vol. 1 Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., Advanced Organic Chemistry, Third Edition, John Wiley & Sons, New York, 1985; Comprehensive Organic Synthesis. Selectivity, Strategy & Efficiency in Modern Organic Chemistry (9 Volume set) Barry M. Trost, Editor-in-Chief, Pergamon Press, New York, 1993.

A number of exemplary methods for the preparation of the compounds of the invention are provided herein. These methods are intended to illustrate the nature of such preparations are not intended to limit the scope of applicable methods.

Deliberate use may be made of protecting groups to mask reactive functionality and direct reactions regioselectively (Greene, etal (1991) Protective Groups in Organic Synthesis 2nd Ed., John Wiley & Sons). For example, useful protecting groups for the 8-hydroxyl group and other hydroxyl substituents include methyl, MOM (methoxymethyl), trialkylsilyl, benzyl, benzoyl, trityl, and tetrahydropyranyl. Certain aryl positions may be blocked from substitution, such as the 2-position as fluorine.

Dihydroxypyrimidine carboxamide (WO 03/035076A1) and N-substituted hydroxypyrimidinone carboxamide (WO 03/035077A1) compounds have been prepared.

### Preparation of Formula Ia-d and Formula IIa-d Phosphonate Esters.

Structures of exemplary pyrimidine Formula I phosphonate esters Ia-d are shown in Chart 1. Structures of exemplary pyrimidine Formula II phosphonate esters IIa-d are shown in Chart 2. Ring substituents R¹, R^{2a}, R^{2b}, R³, R⁴, and R⁵ are as previously defined. Phosphonate ester substituent R^{x} is as previously defined. Compounds of Formula Ia-d and Formula IIa-d may each be an active pharmaceutical ingredient, or an intermediate for preparing other compounds of the invention by subsequent chemical modifications.

Compounds of Formula Ia-d and Formula IIa-d incorporate a phosphonate group (R¹O)₂P(O) connected to the pyrimidine and pyrimidinone scaffold, respectively, by means of a divalent and variable linking group, designated as “L” in the attached structures. Charts 3 and 4 illustrates examples of the phosphonate linking groups (L-A³) present in the structures Ia-d and IIa-d.

The methods described for the introduction of phosphonate substituents are, with modifications made by one skilled in the art, transferable within the phosphonate esters Ia-d and IIa-d. For example, reaction sequences which produce the phosphonates Ia are, with appropriate modifications, applicable to the preparation of the phosphonates Ib-d and IIa-d. Methods described below for the attachment of phosphonate groups by means of reactive substituents such as OH, Br, NH₂, CH₃, CH₂Br, COOH, CHO etc are applicable to each of the scaffolds Ia-d and IIa-d.

### Chart 1. Structures of the Pyrimidine Phosphonates Ia-d

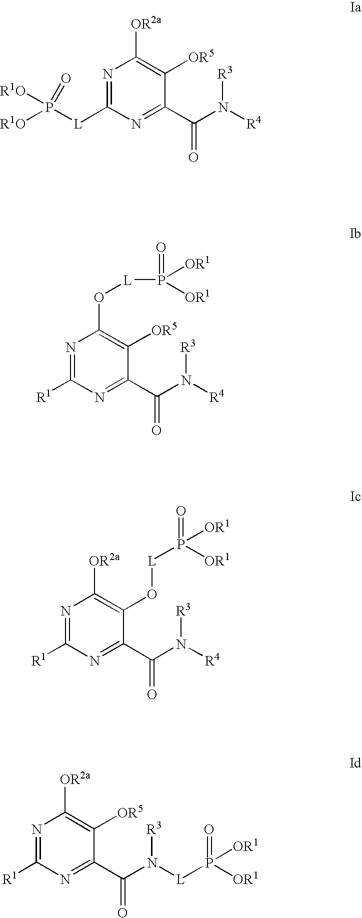

### Chart 2. Structures of Pyrimidinone Phosphonates IIa-d

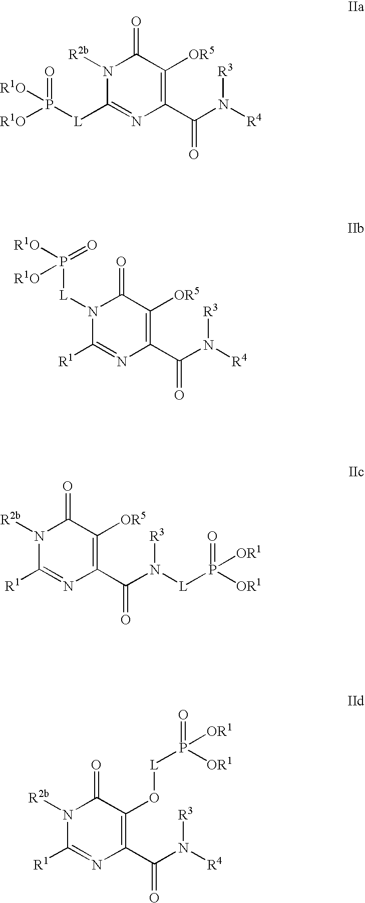

### Chart 3. Examples of Phosphonate Linkages L-A³

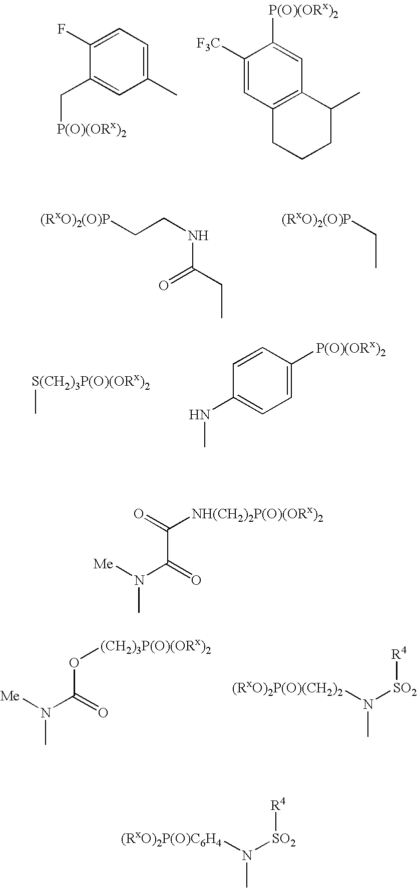

### Chart 4. Examples of Phosphonate Linkages L-A³

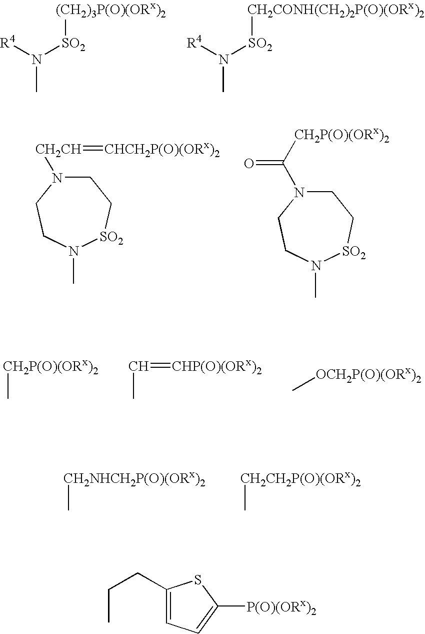

Schemes 1-31 illustrate the syntheses of the phosphonate compounds of this invention, Formulas I and II, and of the intermediate compounds necessary for their synthesis.

Scheme 32 illustrates methods for the interconversion of phosphonate diesters, monoesters and acids, and Scheme 33 illustrates methods for the preparation of carbamates. Schemes 34-37 illustrate the conversion of phosphonate esters and phosphonic acids into carboalkoxy-substituted phosphondiamidates, phosphonamidates, phosphonate monoesters, phosphonate diesters. Scheme 38 illustrates further synthesis of gem-dialkyl amino phosphonate reagents for preparation of Formulas I and II compounds.

### Protection of Reactive Substituents.

Depending on the reaction conditions employed, it may be necessary to protect certain reactive substituents from unwanted reactions by protection before the sequence described, and to deprotect the substituents afterwards, according to the knowledge of one skilled in the art. Protection and deprotection of functional groups are described, for example, in Protective Groups in Organic Synthesis, by T. W. Greene and P. G. M. Wuts, Wiley, Second Edition 1990. Reactive substituents which may be protected are shown in the accompanying schemes as, for example, [OH], [SH], [NH] etc. Protecting groups are also exemplified as “PG”. The selection of a suitable stage in the synthetic sequence for the introduction of the phosphonate group is made by one skilled in the art, depending on the reactivity and stability of the substrates in a given reaction sequence.

### Protection of Phosphonate Esters

Scheme 3a depicts the preparation of phosphonate esters Id and IId in which the phosphonate group is directly attached to the group Ar. In this procedure, a bromo-substituted amine 3.1, in which Ar is an aromatic or heteroaromatic group, is reacted, in the presence of a palladium catalyst, with a dialkyl phosphite 3.2 to yield the aryl phosphonate 3.3. The preparation of arylphosphonates by means of a coupling reaction between aryl bromides and dialkyl phosphites is described in J. Med. Chem., 35, 1371, 1992. This reaction is performed in an inert solvent such as toluene, in the presence of a base such as triethylamine and a palladium (0) catalyst such as tetrakis(triphenylphosphine)palladium(0). Optionally, the amine group is protected prior to the coupling reaction, and deprotected afterwards.

Amine reagent 3.3 is reacted with the ester 3.4 to afford the amide 3.5, and with the ester 3.6 to afford the amide 3.7. The conversion of esters into amides is described in Comprehensive Organic Transformations, by R. C. Larock, VCH, 1989, p. 987. The reactants are combined in a solvent such as toluene or xylene, in the presence of a base such as sodium methoxide under azeotropic conditions, or of a diallyl aluminum or trialkyl tin derivative of the amine. The use of trimethylaluminum in the conversion of esters to amides is described in J. Med. Chem. Chim. Ther., 34, 1999, 1995, and Syn. Comm., 25, 1401, 1995. The reaction is conducted in an inert solvent such as dichloromethane or toluene. The conversion of esters such as 3.4 and 3.6, or the corresponding carboxylic acids, into amides is described in WO 03035077 A1, Optionally, the 5-hydroxyl group of the ester 3.4 and 3.6 is protected, for example as a p-toluenesulfonyl derivative, prior to reaction with the amine component 3.3.

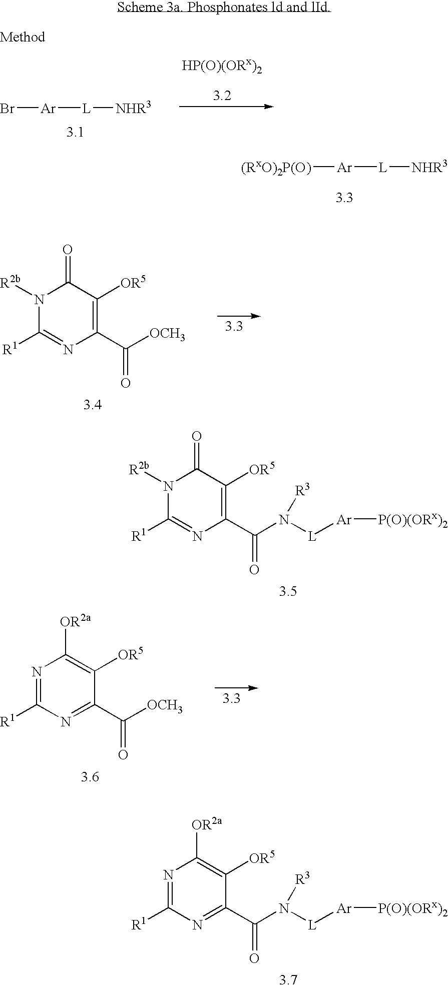

For example, 3-bromo-4-fluorobenzylamine 3.8 (Lancaster) is reacted in toluene solution at ca 100° C., with one molar equivalent of a dialkyl phosphite 3.9, triethylamine and 3 mol % of tetrakis(triphenylphosphine)palladium(0), to give the phosphonate product 3.10 in Scheme 3b. Compound 3.10 is then reacted, in toluene solution at reflux temperature with 3.11 to yield the pyrimidine amide 3.12. Alternatively, 3.10 is reacted, in toluene solution at reflux temperature with 3.13 to yield the pyrimidinone amide 3.14

Using the above procedures, but employing, in place of the amine 3.8, different amines 3.1, and/or different esters 3.4, the corresponding amides 3.5 are obtained.

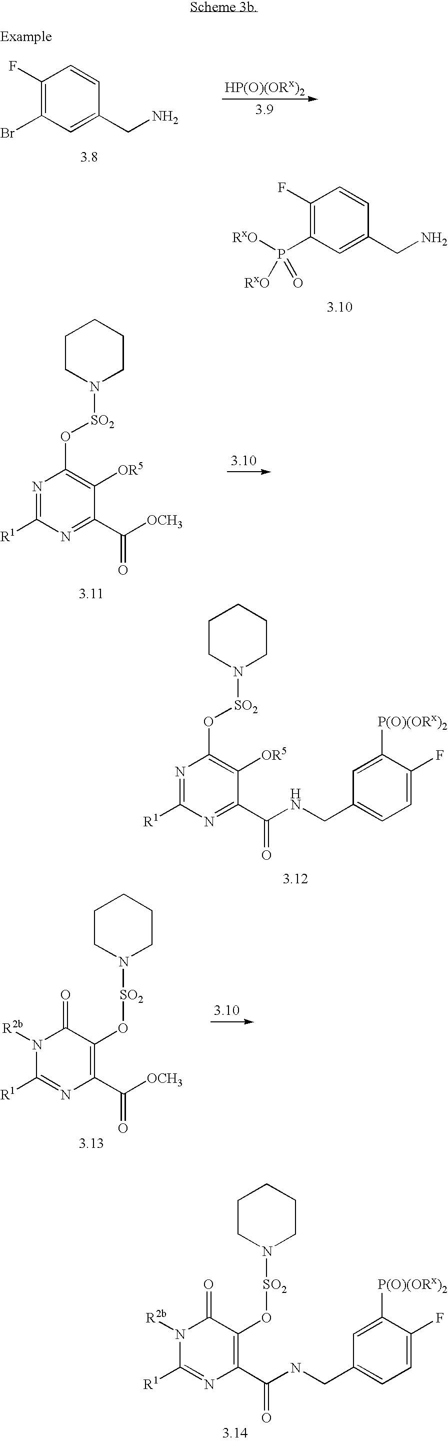

Scheme 4 depicts the preparation of phosphonate esters 1 in which the phosphonate group is attached by means of a saturated or unsaturated alkylene chain. In this procedure, a bromo-substituted amine 4.1, in which Ar is an aryl or heterocycle group, is subjected to a Heck coupling reaction, in the presence of a palladium catalyst, with a dialkyl alkenyl phosphonate 4.2, in which R^{5a} is a direct bond, a divalent group such as alkylene, alkenylene, alkynylene or cycloalkylene group, optionally incorporating a heteroatom O, S or N, ethyleneoxy, polyethyleneoxy, or a functional group such as an amide, ester, oxime, sulfoxide or sulfone etc, or an optionally substituted aryl, heterocycle or aralkyl group, to give the amine 4.3. The coupling of aryl halides with olefins by means of the Heck reaction is described, for example, in Advanced Organic Chemistry by F. A. Carey and R. J. Sundberg, Plenum, 2001, p. 503ff and in Acc. Chem. Res., 12, 146, 1979. The aryl bromide and the olefin are coupled in a polar solvent such as dimethylformamide or dioxane, in the presence of a palladium(0) catalyst such as tetrakis(triphenylphosphine)palladium(0) or a palladium(II) catalyst such as palladium(II) acetate, and optionally in the presence of a base such as triethylamine or potassium carbonate. Optionally, the amine substituent is protected prior to the coupling reaction, and deprotected afterwards. The phosphonate amine 4.3 is then coupled, as described above, with the ester 4.4, or the corresponding carboxylic acid, to produce the amide 4.5. Optionally, the double bond is reduced to give the saturated analog 4.6. The reduction of olefinic bonds is described in Comprehensive Organic Transformations. by R. C. Larock, VCH, 1989, p. 6ff. The transformation is effected by means of catalytic hydrogenation, for example using a palladium on carbon catalyst and hydrogen or a hydrogen donor, or by the use of diimide or diborane.

For example, 3-bromo-4-methoxybenzylamine 4.7 (Lancaster) is reacted in dioxane solution with one molar equivalent of a diallyl vinyl phosphonate 4.8 (Aldrich) and potassium carbonate, to yield the olefinic phosphonate 4.9. The product is then reacted, as described above, with 6-methyl ester 4.10, prepared as described in Scheme 1A, to give the amide 4.11. The latter compound is reacted with diimide, prepared by basic hydrolysis of diethyl azodicarboxylate, as described in Angew. Chem. Int. Ed., 4, 271, (1965), to yield the saturated product 4.12.

Using the above procedures, but employing, in place of the amine 4.7, different amines 4.1, and/or different phosphonates 4.2, and/or different bicyclic esters 4.4, the corresponding amides 4.5 and 4.6 are obtained.

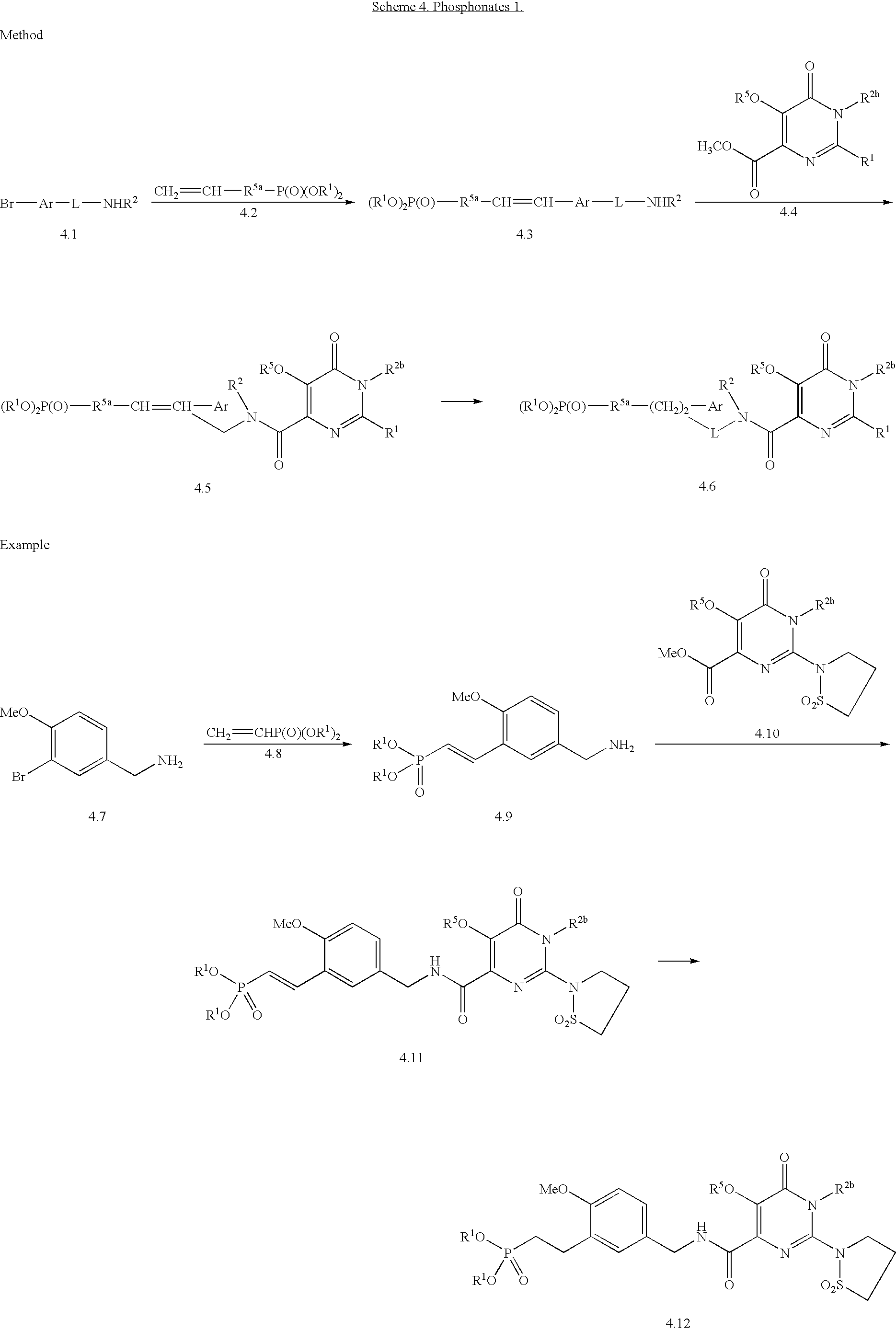

Scheme 5 depicts the preparation of phosphonate esters Id in which the phosphonate group is attached by means of an amide linkage. In this procedure, the amine group, of a carboxy-substituted amine 5.1 is protected to afford the derivative 5.2. The protection of amino groups is described in Protective Groups in Organic Synthesis, by T. W. Greene and P. G. M. Wuts, Wiley, Second Edition 1990, p. 309ff. Amino groups are protected, for example by alkylation, such as by mono or dibenzylation, or by acylation. The conversion of amines into mono or dibenzylamines, for example by treatment with benzyl bromide in a polar solvent such as acetonitrile or aqueous ethanol, in the presence of a base such as triethylamine or sodium carbonate, is described in Protective Groups in Organic Synthesis, by T. W. Greene and P. G. M. Wuts, Wiley, Second Edition 1990, p. 364. The N-protected carboxylic acid 5.2 is then coupled with an amino-substituted dialkyl phosphonate 5.3, in which the group R^{5a} is as defined in Scheme 4, to yield the amide 5.4. The preparation of amides from carboxylic acids and derivatives is described, for example, in Organic Functional Group Preparations, by S. R. Sandier and W. Karo, Academic Press, 1968, p. 274, and in Comprehensive Organic Transformations, by R. C. Larock, VCH, 1989, p. 972ff. The carboxylic acid is reacted with the amine in the presence of an activating agent, such as, for example, dicyclohexylcarbodiimide or diisopropylcarbodiimide, optionally in the presence of, for example, hydroxybenzotriazole, N-hydroxysuccinimide or N-hydroxypyridone, in a non-protic solvent such as, for example, pyridine, DMF or dichloromethane, to afford the amide.

Alternatively, the carboxylic acid is first converted into an activated derivative such as the acid chloride, anhydride, mixed anhydride, imidazolide and the like, and then reacted with the amine, in the presence of an organic base such as, for example, pyridine, to afford the amide.

The conversion of a carboxylic acid into the corresponding acid chloride is effected by treatment of the carboxylic acid with a reagent such as, for example, thionyl chloride or oxalyl chloride in an inert organic solvent such as dichloromethane, optionally in the presence of a catalytic amount of dimethylformamide.

The amino-protecting group is then removed from the product 5.4 to give the free amine 5.5. Deprotection of amines is described in Protective Groups in Organic Synthesis, by T. W. Greene and P. G. M. Wuts, Wiley, Second Edition 11990, p. 309ff. The amine is then coupled with the carboxylic acid 5.6, as described above, to produce the amide 5.7.

For example, 4-carboxycyclohexylmethylamine 5.8 (Aldrich) is converted into the phthalimido derivative 5.9 (pht=phthalimide). The conversion of amines into phthalimido derivatives is described in Protective Groups in Organic Synthesis by T. W. Greene and P. G. M. Wuts, Wiley, Second Edition 1990, p. 358. The conversion is effected by reaction of the amine with an equimolar amount of 2-carbomethoxybenzoyl chloride, N-carboethoxyphthalimide, or preferably, phthalic anhydride. The reaction is performed in an inert solvent such as toluene, dichloromethane or acetonitrile, to prepare the phthalimido derivative 5.9. This material is then reacted with one molar equivalent of a dialkyl aminoethyl phosphonate 5.10, (J. Org. Chem., (2000), 65, 676) and dicyclohexylcarbodiimide in dimethylformamide, to give the amide 5.11. The phthalimido protecting group is then removed, for example by reaction with ethanolic hydrazine at ambient temperature, as described in J. Org. Chem., 43, 2320, (1978), to afford the amine 5.12. This compound is coupled in dimethylformamide solution with 6-carboxylic acid 5.13, to afford the amide 5.14.

Using the above procedures, but employing, in place of the amine 5.8, different amines 5.1, and/or different phosphonates 5.3, and/or different carboxylic acids 5.6, the corresponding products 5.7 are obtained.

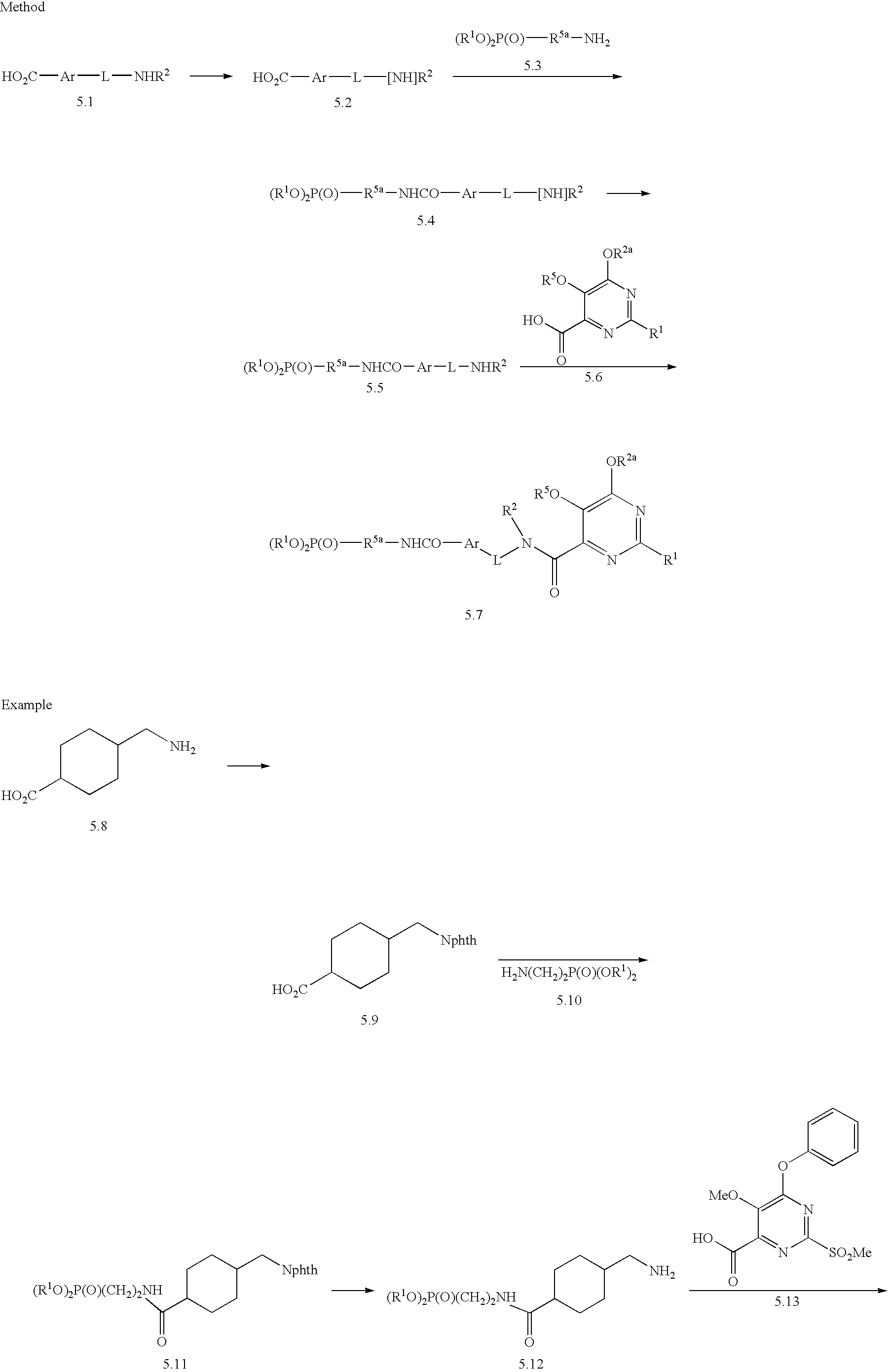

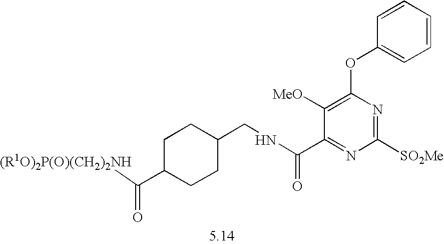

Scheme 6 depicts the preparation of phosphonates IId in which the phosphonate is attached by means of an ether linkage. In this procedure, the amino group of a hydroxy-substituted amine 6.1 may be protected (PG=protecting group), as described above, to give the derivative 6.2. The carbinol is then reacted, with base catalysis, with a dialkyl bromomethyl phosphonate 6.3, in which the group R⁵ is as defined in Scheme 4. The reaction is conducted in a polar aprotic solvent such as tetrahydrofuran, dimethylformamide or dimethylsulfoxide, in the presence of a base such as potassium carbonate, for cases in which Ar is an aromatic group, or a strong base such as sodium hydride, for cases in which Ar is an aliphatic group. The amino group of the resulting ether 6.4 is then deprotected, as previously described, to give the amine 6.5. The amine is then reacted with the ester 6.6, as described in Scheme 3, to give the amide 6.7.

For example, N-methyl 3-hydroxyphenethylamine 6.8 is reacted with one molar equivalent of acetyl chloride in dichloromethane containing pyridine, to give the N-acetyl product 6.9. The product is then reacted at ca. 60° C. in dimethylformamide (DMF) solution with one molar equivalent of a diallyl 3-bromopropenyl phosphonate 6.10 (Aurora) and cesium carbonate, to produce the ether 6.11. The N-acetyl group is then removed, for example by treatment with hog kidney acylase, as described in Tetrahedron, 44, 5375, (1988), to give the amine 6.12. The product is then reacted in toluene solution at reflux, 6.13, to yield the amide 6.14.

Using the above procedures, but employing, in place of the amine 6.8, different amines 6.1, and/or different phosphonates 6.3, and/or different bicyclic esters 6.6, the corresponding products 6.7 are obtained.

Scheme 7 depicts the preparation of phosphonates IId in which the phosphonate is attached by means of an ether or thioether linkage. In this procedure, a N-protected hydroxyamine 6.2, in which Ar is an aromatic moiety, is subjected to a Mitsunobu reaction with a hydroxy or mercapto-substituted dialkyl phosphonate 7.1, in which R^{5a} is as defined in Scheme 4, to prepare the ether or thioether product 7.2. The preparation of aromatic ethers and thioethers by means of the Mitsunobu reaction is described, for example, in Comprehensive Organic Transformations, by R. C. Larock, VCH, 1989, p. 448, and in Advanced Organic Chemistry, Part B, by F. A. Carey and R. J. Sundberg, Plenum, 2001, p. 153-4 and in Org. React., 1992, 42, 335. The phenol and the alcohol or thiol component are reacted together in an aprotic solvent such as, for example, tetrahydrofuran or dioxane, in the presence of a dialkyl azodicarboxylate and a triarylphosphine, to afford the ether or thioether products. The N-protecting group is then removed and the resultant amine is converted, as described in Scheme 6, into the amide 7.3.

For example, N-acetyl 3,5-dichloro-4-hydroxybenzylamine 7.4 is reacted in a tetrahydrofuran solution with one molar equivalent of a dialkyl mercaptoethyl phosphonate 7.5, (Zh. Obschei. Khim., 1973, 43, 2364) diethyl azodicarboxylate and tri-o-tolylphosphine, to afford the thioether product 7.6. The N-acetyl group is removed, as described in Scheme 6, and the amine 7.7 is then reacted with methyl ester 7.8 (TBDMS=tert-butyldimethylsilyl), to afford the amide 7.9.

Using the above procedures, but employing, in place of the amine 7.4, different amines 6.2, and/or different phosphonates 7.2, the corresponding products 7.3 are obtained.

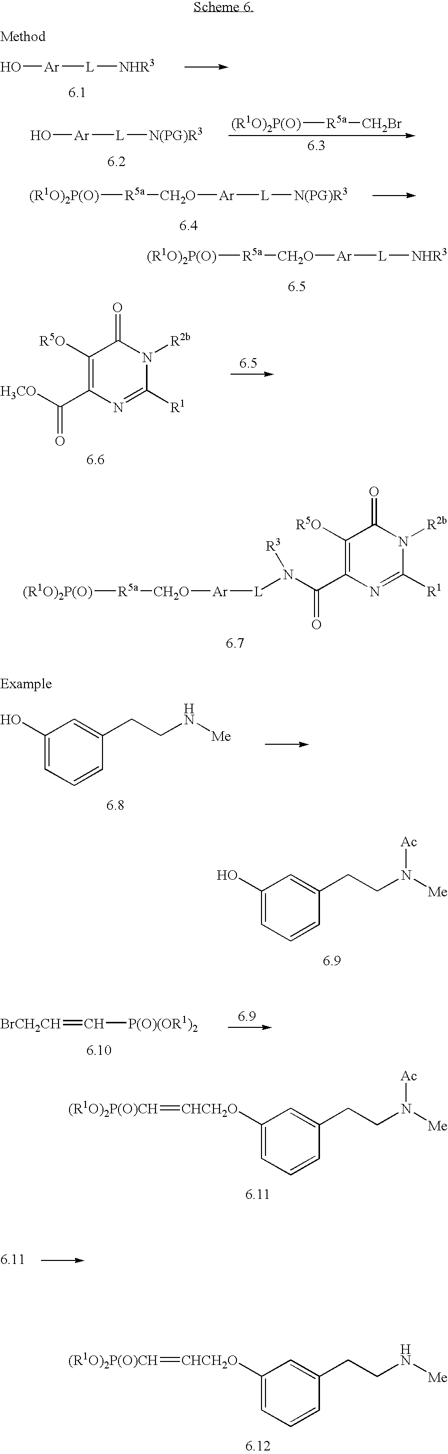

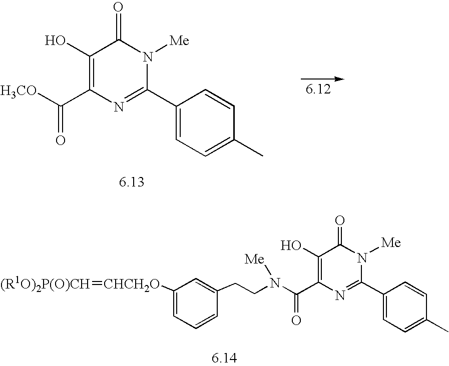

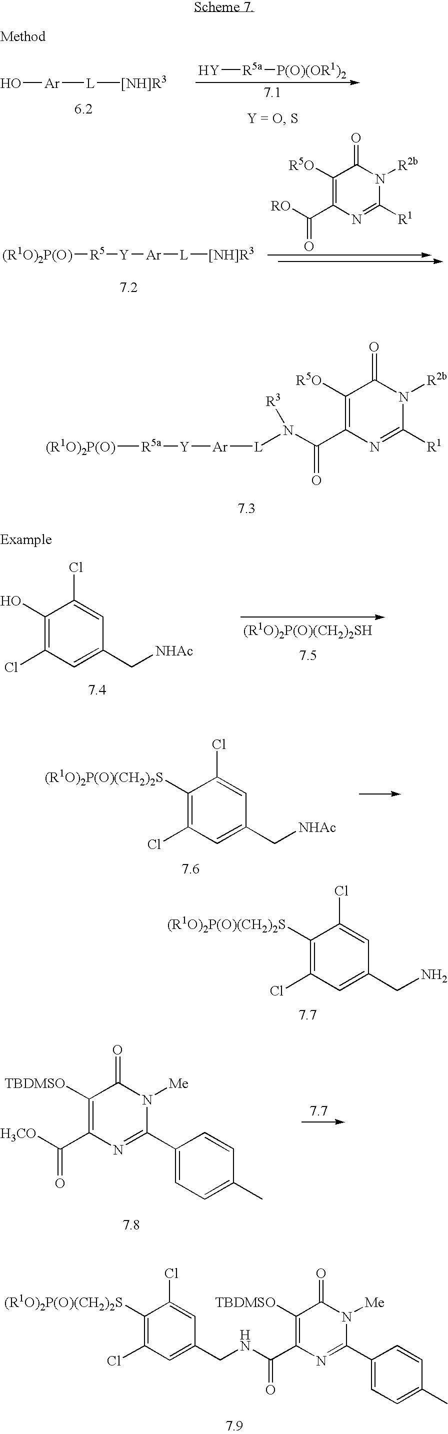

Scheme 8 depicts the preparation of phosphonates Id in which the phosphonate is attached by means of an alkylene chain incorporating an amide linkage. In this procedure, an amine 8.1 is reacted with a bromoalkyl ester 8.2, in which R^{5a} is as defined in Scheme 4, to yield the alkylated amine 8.3. The preparation of substituted amines by the reaction of amines with alkyl halides is described, for example, in Comprehensive Organic Transformations, by R. C. Larock, VCH, 1989, p. 397. Equimolar amounts of the reactants are combined in a polar solvent such as an alkanol or dimethylformamide and the like, in the presence of a base such as cesium carbonate, diazabicyclononene or dimethylaminopyridine, to yield the substituted amine. The ester group is then hydrolyzed to give the carboxylic acid 8.4, and this compound is then coupled, as described in Scheme 5, with a diallyl aminoalkyl phosphonate 8.5, to produce the aminoamide 8.6. Optionally, the amino group of the amine 8.4 is protected prior to the coupling reaction, and deprotected afterwards. The product is then reacted with the bicyclic hydroxyester 8.7 to afford the amide 8.8.

For example, 4-trifluoromethylbenzylamine 8.9 is reacted in dimethylformamide with one molar equivalent of methyl bromoacetate 8.10 and potassium carbonate to give the ester 8.11. Hydrolysis, employing one molar equivalent of lithium hydroxide in aqueous dimethoxyethane, affords the carboxylic acid 8.12, and this compound is coupled in tetrahydrofuran solution with a dialkyl aminomethyl phosphonate 8.13 (Aurora), in the presence of dicyclohexylcarbodiimide, to give the aminoamide 8.14. The product is then reacted with 4-sulfonamide, 6-methyl ester 8.15, prepared by the methods described above, to yield the amide 8.16.

Using the above procedures, but employing, in place of the amine 8.9, different amines 8.1, and/or different bromoesters 8.2, and/or different phosphonates 8.5, and/or different hydroxyesters 8.7, the corresponding products 8.8 are obtained.

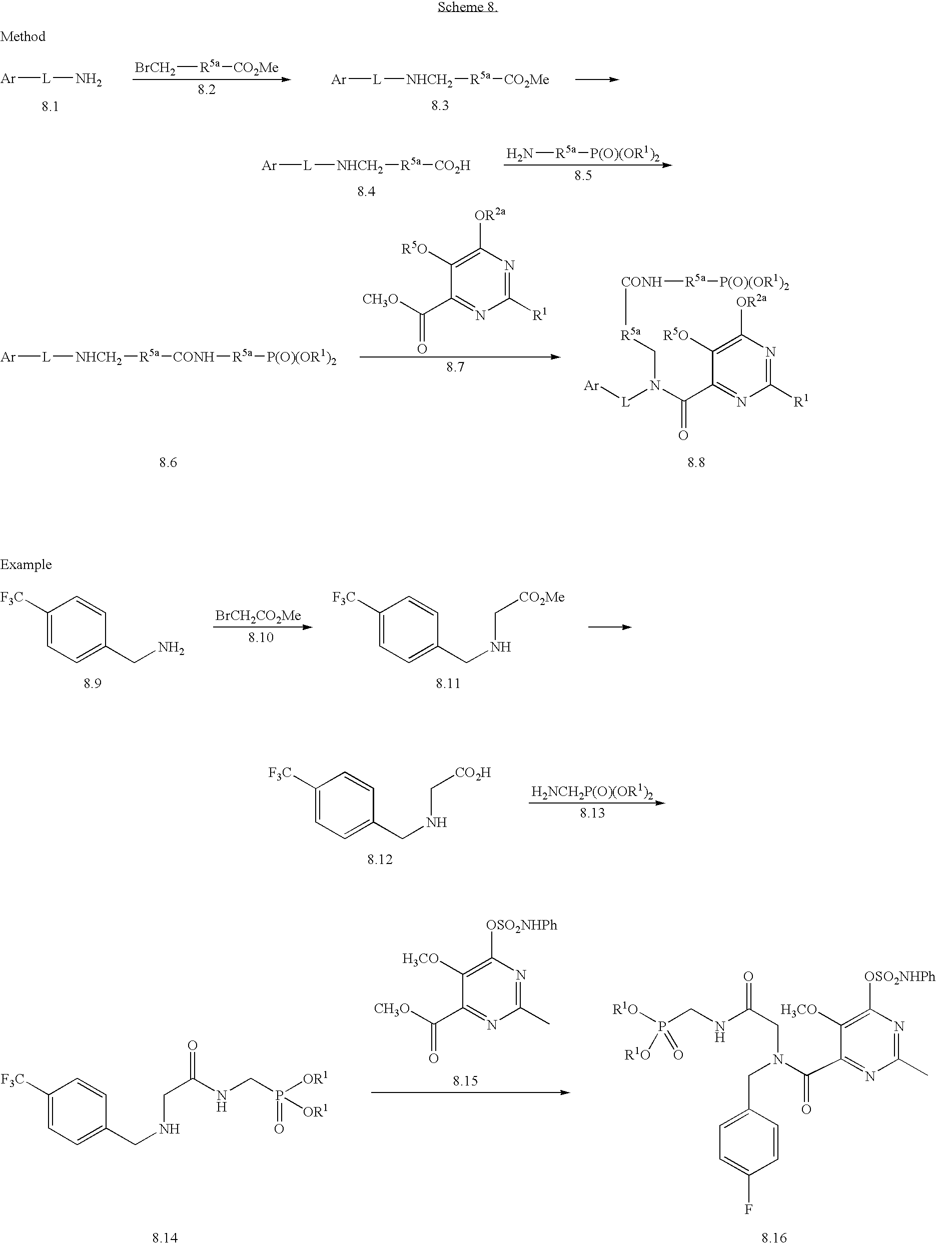

Scheme 9 depicts the preparation of phosphonates IId in which the phosphonate is attached by means of a variable carbon chain. In this procedure, a primary amine 9.1 is subjected to a reductive amination reaction with a diallyl formyl-substituted phosphonate 9.2, in which R⁵ is as defined in Scheme 4, to afford the alkylated amine 9.3. The preparation of amines by means of reductive amination procedures is described, for example, in Comprehensive Organic Transformations, by R. C. Larock, VCH, p. 421, and in Advanced Organic Chemistry, Part B, by F. A. Carey and R. J. Sundberg, Plenum, 2001, p. 269. In this procedure, the amine component and the aldehyde or ketone component are reacted together in a polar solvent in the presence of a reducing agent such as, for example, borane, sodium cyanoborohydride, sodium triacetoxyborohydride or diisobutylaluminum hydride, optionally in the presence of a Lewis acid, such as titanium tetraisopropoxide, as described in J. Org. Chem., 55, 2552, 1990. The product 9.3 is then reacted, as described previously, with the bicyclic ester 9.4 to give the amide 9.5.

For example, 3,4-dichlorobenzylamine is reacted in methanol solution with one molar equivalent of a dialkyl 3-formylphenyl phosphonate 9.7, (Epsilon) and sodium cyanoborohydride, to yield the allylated product 9.8. This compound is then reacted with 2-dimethylcarbamoyl-5,6-dihydroxy-pyrimidine-4-carboxylic acid methyl ester 9.9, prepared using the methods described above, from the corresponding bromo compound and N-methyl methanesulfonamide, to give the amide 9.10.

Using the above procedures, but employing, in place of the amine 9.6, different amines 9.1, and/or different phosphonates 9.2, and/or different bicyclic esters 9.4, the corresponding products 9.5 are obtained.

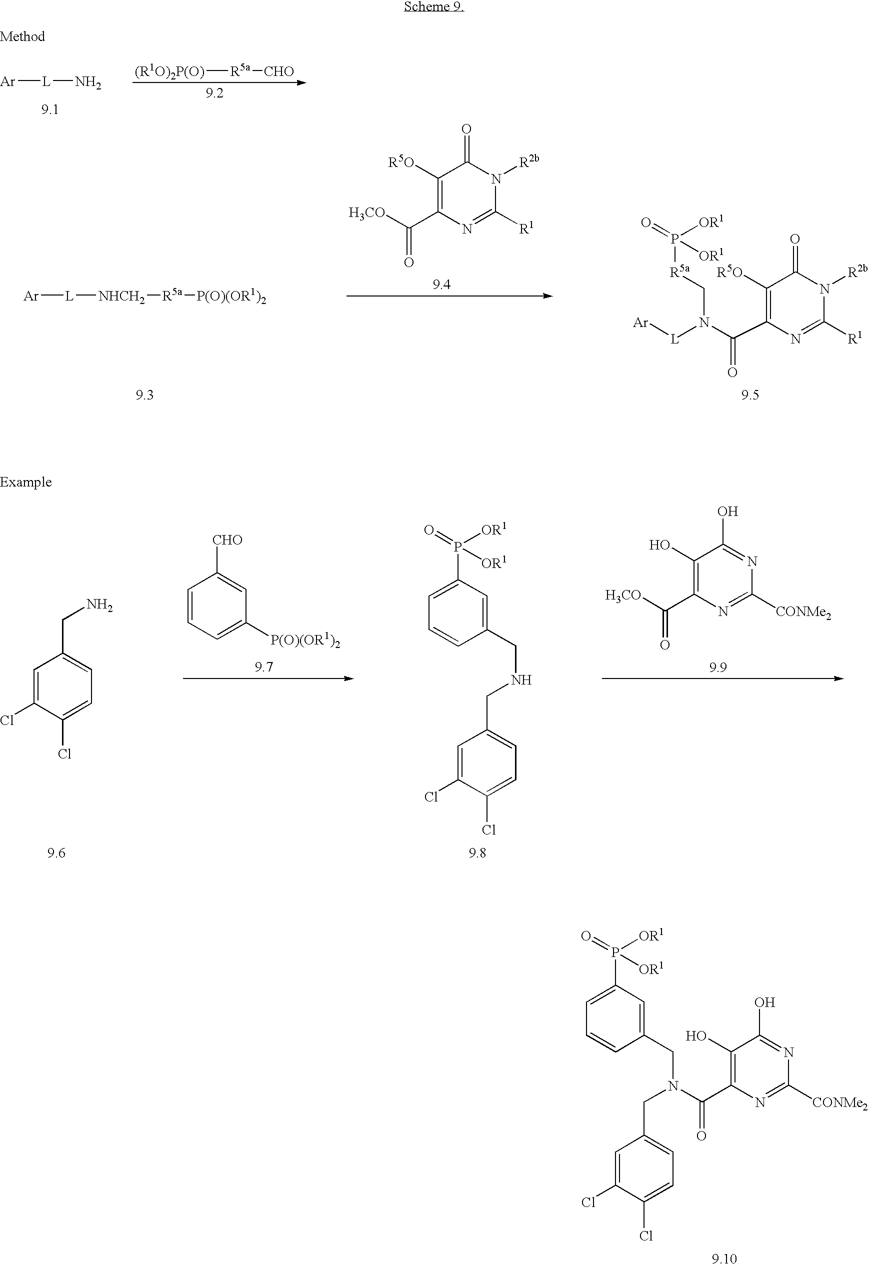

Scheme 10 depicts an alternative method for the preparation of phosphonates IId in which the phosphonate is attached by means of a variable carbon chain. In this procedure, the phenolic group of a bicyclic amide 10.1, prepared as described above, and in WO 02 30930 A2, is protected to give the product 10.2. The protection of phenolic hydroxyl groups is described in Protective Groups in Organic Synthesis, by T. W. Greene and P. G. M. Wuts, Wiley, Second Edition 1990, p. 10ff. For example, hydroxyl substituents are protected as trialkylsilyloxy ethers. Trialkylsilyl groups are introduced by the reaction of the phenol with a chlorotrialkylsilane and a base such as imidazole, for example as described in Protective Groups in Organic Synthesis, by T. W. Greene and P. G. M. Wuts, Wiley, Second Edition 1990, p. 10, p. 68-86. Alternatively, phenolic hydroxyl groups are protected as benzyl or substituted benzyl ethers, or as acetal ethers such as methoxymethyl or tetrahydropyranyl. The O-protected amide 10.2 is then reacted with the phosphonate-substituted trifluoromethanesulfonate 10.3, in which R^{5a} is as defined in Scheme 4, to produce the allylated amide 10.4. The alkylation reaction is conducted between equimolar amounts of the reactants in an aprotic organic solvent such as dimethylformamide or dioxane, in the presence of a strong base such as lithium hexamethyl disilylazide or sodium hydride, at from ambient temperature to about 90° C. The hydroxyl group is then deprotected to give the phenol 10.5. Deprotection of phenolic hydroxyl groups is described in Protective Groups in Organic Synthesis, by T. W. Greene and P. G. M. Wuts, Wiley, Second Edition 1990, p. 1 Off. For example, silyl protecting groups are removed by reaction with tetrabutylammonium fluoride, benzyl groups are removed by catalytic hydrogenation and acetal ethers are removed by treatment with acids.

Amide 10.7 is reacted with one molar equivalent of tert-butyl chlorodimethylsilane and imidazole in dichloromethane, to give 5-(tert-butyl-dimethyl-silanyloxy)-1-methyl-6-oxo-2-phenyl-1,6-dihydro-pyrimidine-4-carboxylic acid (naphthalen-2-ylmethyl)-amide 10.8. This compound 10.8 is then reacted at ambient temperature in dioxane solution with one molar equivalent of sodium hydride, followed by the addition of a dialkyl trifluoromethanesulfonyloxymethyl phosphonate. 10.9 (Tet. Lett, 1986, 27, 1477), to afford the alkylated product 10.10. Deprotection, by reaction with tetrabutylammonium fluoride in tetrahydrofuran, then yields the product 10.11.

Using the above procedures, but employing, in place of the amide 10.7, different amides 10.1, and/or different phosphonates 10.3, the corresponding products 10.5 are obtained.

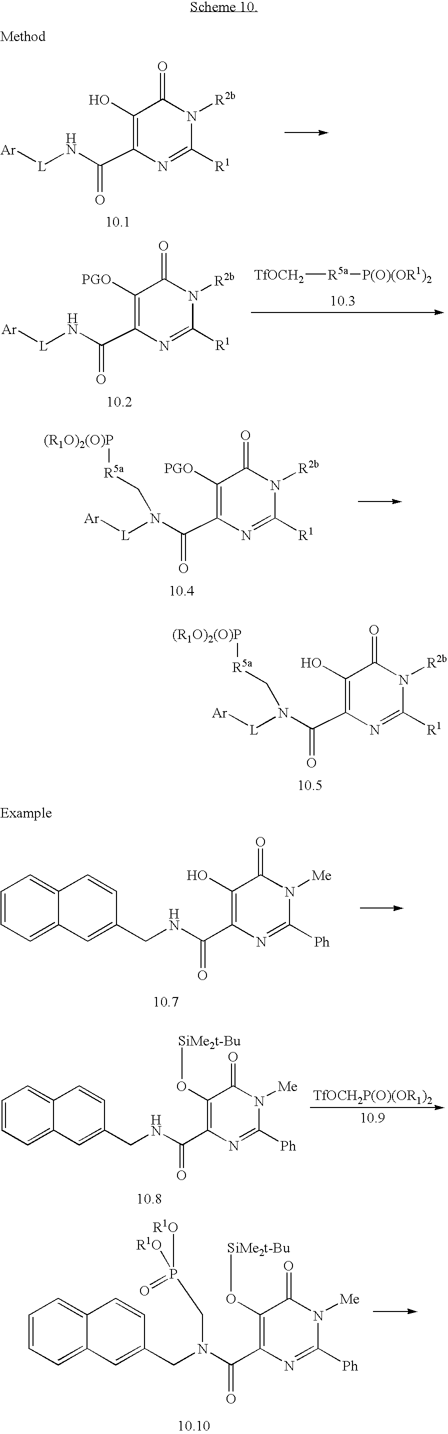

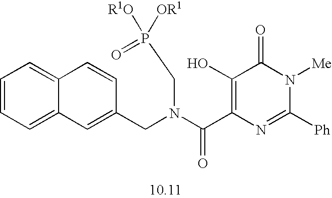

Schemes 11-15 illustrate methods for the preparation of the 2-phosphonate esters Ia and IIa.

Scheme 11 depicts the preparation of 2-substituted pyrimidyl phosphonates Ia in which the phosphonate is attached by means of a heteroatom O, S or N, and a variable carbon chain. In this procedure, an amide 11.1, prepared as previously described, is reacted in an aprotic solvent such as dichloromethane, hexachloroethane or ethyl acetate with a free radical brominating agent such as N-bromosuccinimide or N-bromoacetamide, to yield the 5-bromo product 11.2. This compound is then reacted with a diallyl hydroxy, mercapto or amino-substituted phosphonate 11.3, in which R⁵ is as defined as in Scheme 4, to give the ether, thioether or amine product 11.4. The displacement reaction is conducted in a polar aprotic organic solvent such as dimethylformamide or DMPU, at from 100° C. to about 150° C., in the presence of a base such as triethylamine or cesium carbonate, for example as described in WO 0230930A2, Examples 57-69.

Cyclohexylmethyl-amide 11.6 is reacted with one molar equivalent of N-bromosuccinimide in dichloromethane to yield the 5-bromo product 11.7. This material is then reacted with a dialkyl mercaptoethyl phosphonate 11.8 (Zh. Obschei. Khim., 1973, 43, 2364) and triethylamine at ca 100° C. in a pressure vessel, to produce the thioether 11.9.

Ketal protected 11.11 is brominated with N-bromosuccinimide in ethyl acetate at reflux temperature to yield the bromo compound 11.12 which is reacted with a dialkyl 3-aminophenyl phosphonate 11.13 (J. Med. Chem., 1984, 27, 654) in dimethylformamide at ca. 130° C., using the procedure described in WO 0230930 A2 Example 63, to give the phosphonate 11.14. The product is then reacted with N,N-dimethyloxamide 11.15, (Japanese Patent 540467 18) and dicyclohexylcarbodiimide in dimethylformamide, to yield the amide product 11.16.

Using the above procedures, but employing, in place of the amides 11.6 or 11.11, different amides 11.1, and/or different phosphonates 11.3, the corresponding products 11.4 are obtained.

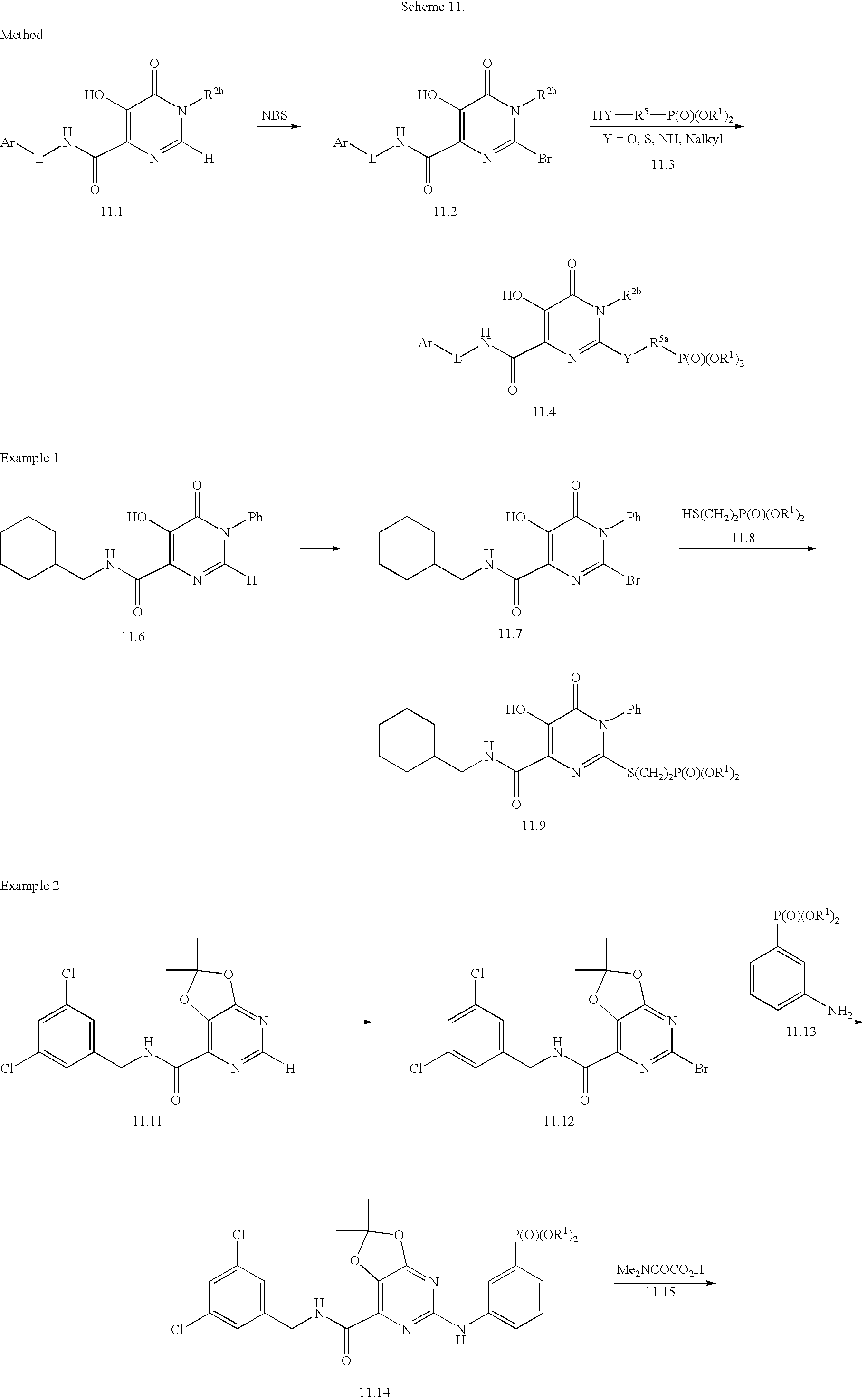

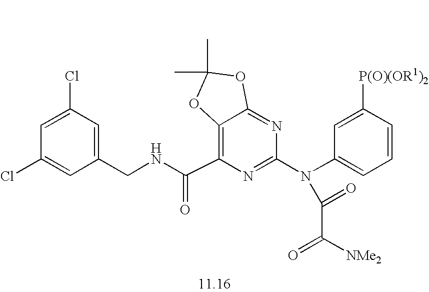

Scheme 12 depicts the preparation of phosphonates IIa in which the phosphonate is attached by means of a carbamate linkage. In this procedure, a protected bromophenol 12.1 is reacted, as described in Scheme 11, with an amine 12.2 to give the displacement product 12.3. This compound is then reacted with phosgene, triphosgene, carbonyl diimidazole or a functional equivalent thereof, and a dialkyl hydroxyalkyl phosphonate 12.4, in which R⁵ is as defined in Scheme 4, to yield, after deprotection of the phenol, the carbamate 12.5. Various methods for the preparation of carbamates are described in Scheme 33.

For example, the hydroxyester 12.6 is converted, as described previously, into the amide 12.7. This material is then reacted, in dimethylformamide solution at 100° C., with ethylamine and cesium carbonate in dimethylformamide, to afford 5-(tert-butyl-dimethyl-silanyloxy)-2-ethylamino-1-methyl-6-oxo-1,6-dihydro-pyrimidine-4-carboxylic acid [2-(4-fluoro-phenyl)-cyclopropyl]-amide 12.9. The amine is treated with equimolar amounts of a dialkyl hydroxypropyl phosphonate 12.10 (Zh. Obschei. Khim., 1974, 44, 1834) and carbonyldiimidazole in dichloromethane, to prepare, after desilylation, the carbamate phosphonate 12.11.

Using the above procedures, but employing, in place of the amide 12.7, different amides 12.3, and/or different phosphonates 12.4, the corresponding products 12.5 are obtained.

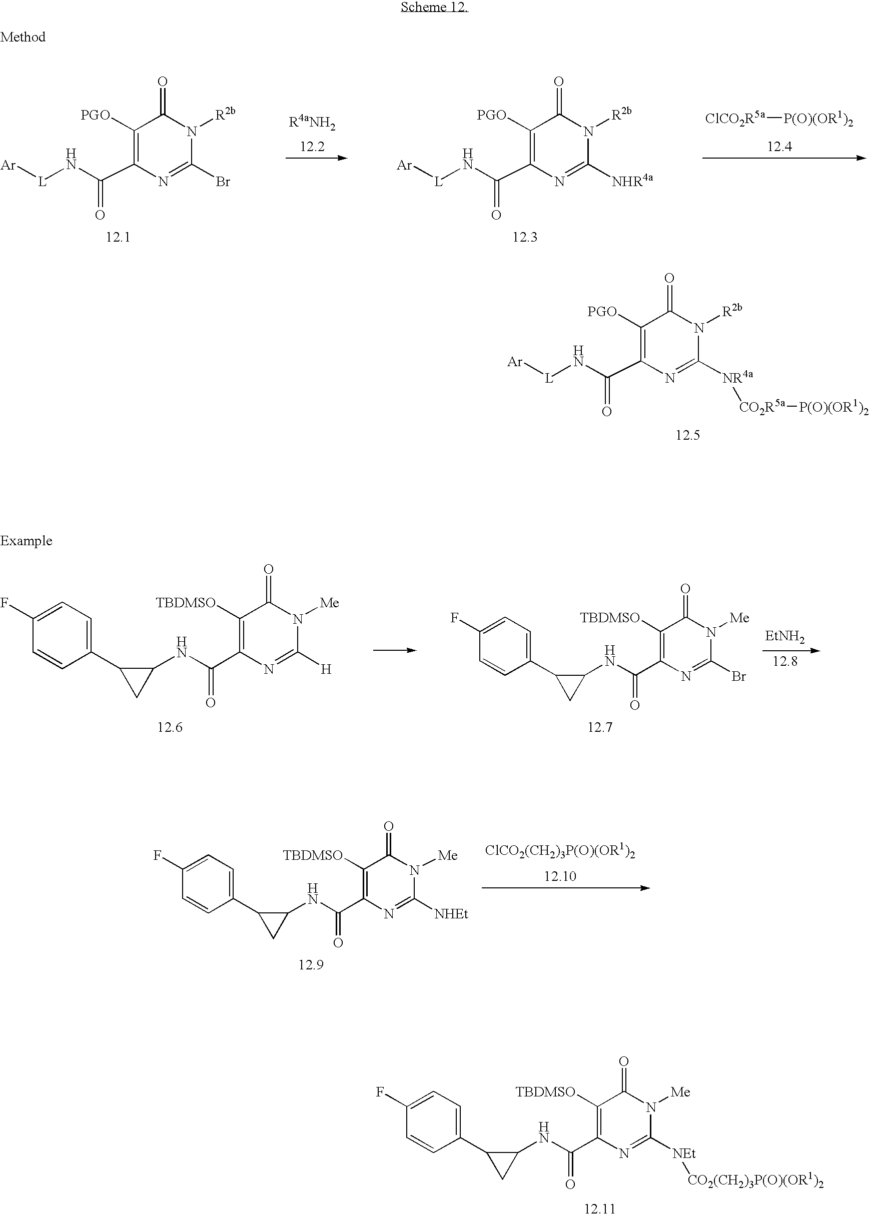

Scheme 13 depicts the preparation of phosphonates IIa in which the phosphonate is attached by means of an arylvinyl or arylethyl linkage. In this procedure, a bromophenol 13.1 is protected to give the product 13.2. This compound is then coupled with tributylvinyltin to yield the 5-vinyl product 13.3. The coupling reaction is effected in dimethylformamide solution at ca 80° C. in the presence of a palladium(0) catalyst, such as tris(dibenzylideneacetone)palladium(0), a triarylphosphine such as tri(2-furyl)phosphine and copper(I) iodide, for example as described in WO 0230930A2, Example 176. The vinyl-substituted product is subjected to a palladium-catalyzed Heck coupling reaction, as described in Scheme 4, with a dibromoaromatic or heteroaromatic compound 13.4, to give the bromoaryl product 13.5. The latter compound is then coupled, as described in Scheme 3, with a dialkyl phosphite 13.6, in the presence of a palladium catalyst, to give the aryl phosphonate 13.7. Deprotection then affords the phenol 13.8. Optionally, the double bond is reduced, for example as described in Scheme 4, to give the saturated analog 13.9.

For example, 5-(tert-butyl-dimethyl-silanyloxy)-1-isopropyl-6-oxo-1,6-dihydro-pyrimidine-4-carboxylic acid 3,5-dichloro-benzylamide 13.10, (WO9944992) is converted, using the methods described above, into 2-bromo-5-(tert-butyl-dimethyl-silanyloxy)-1-isopropyl-6-oxo-1,6-dihydro-pyrimidine-4-carboxylic acid 3,5-dichloro-benzylamide 13.11. The product is coupled, as described above, with tri(n-butyl)vinyltin to produce 2-ethylene-5-(tert-butyl-dimethyl-silanyloxy)-1-isopropyl-6-oxo-1,6-dihydro-pyrimidine-4-carboxylic acid 3,5-dichloro-benzylamide 13.12. This material is then coupled, in dimethylformamide solution at 80° with one molar equivalent of 2,5-dibromothiophene 13.13, in the presence of tetrakis(triphenylphosphine)palladium(0) and triethylamine, to afford 2-[2-(2-bromothiophene)ethylene, 3-isopropyl, 5-tert-butyldimethylsilyloxy, 6-[3,5-dichloro-benzylamide]pyrimidinone 13.14. The product 13.14 is coupled, in the presence of a palladium(0) catalyst and triethylamine, with a diallyl phosphite 13.15, to afford the phosphonate 13.16. Deprotection, for example by reaction with tetrabutylammonium fluoride in tetrahydrofuran, then yields the phenol 13.17, and hydrogenation of the latter compound in methanol, using 5% palladium on carbon as catalyst, produces the saturated analog 13.18.

Using the above procedures, but employing, in place of the amide 13.11, different amides 13.1, and/or different dibromides 13.4, the corresponding products 13.8 and 13.9 are obtained.

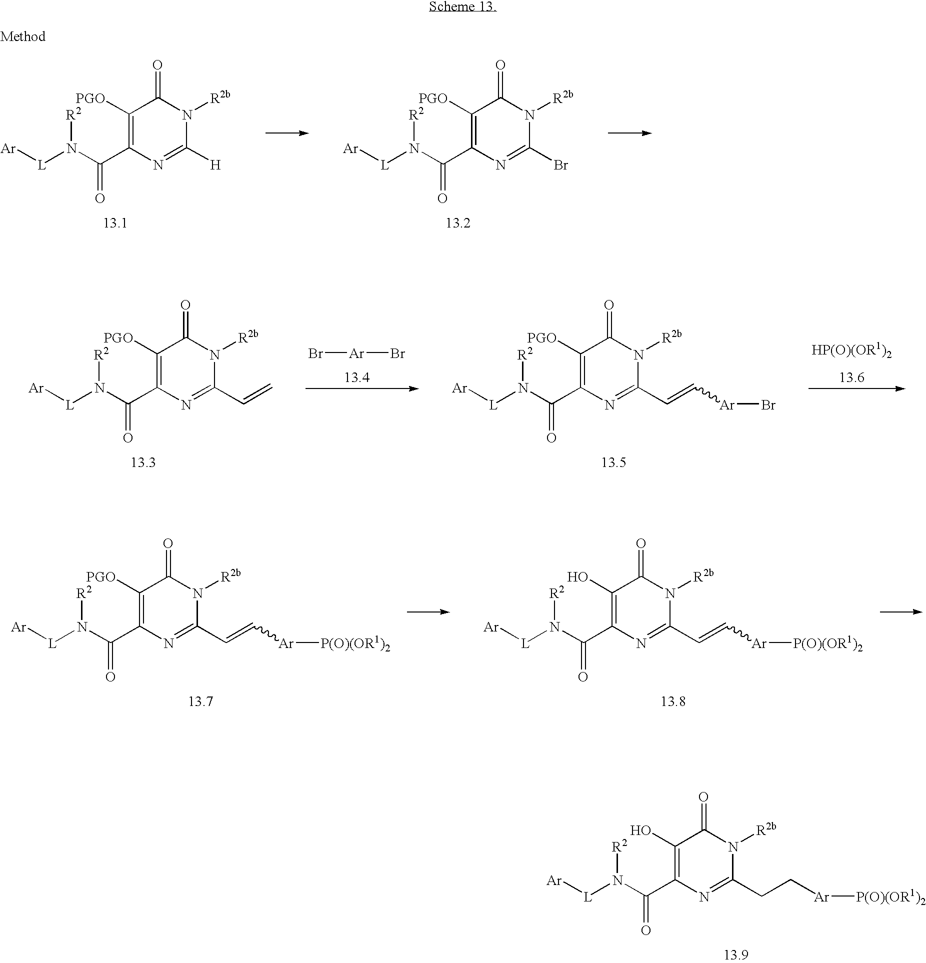

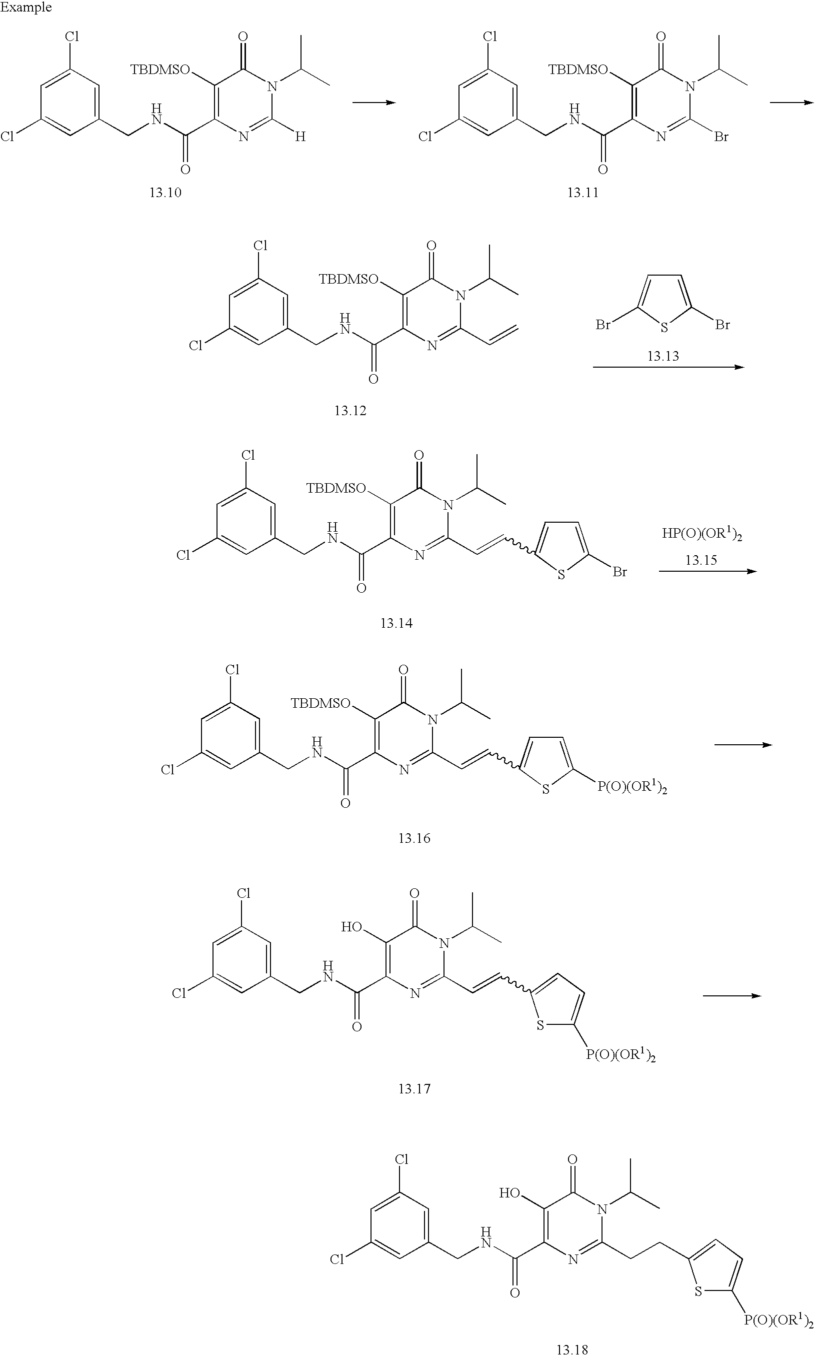

Scheme 14 depicts the preparation of phosphonates Ia in which the phosphonate is attached by means of an acetylenic bond. In this procedure, a phenol 14.1 is reacted, as described in WO 0230930 A2 p. 166 and Example 112, with N-iodosuccinimide in dichloromethane-dimethylformamide, to give the 5-iodo product; protection of the phenolic hydroxyl group then affords the compound 14.2. This material is coupled, as described in WO 0230930 A2 Example 79, in dimethylformamide solution, in the presence of dichlorobis(triphenylphosphine) palladium (II), copper iodide and triethylamine, with a diallyl ethynyl phosphonate 14.3, in which R^{5a} is as defined in Scheme 4, to give, after deprotection of the phenol, the acetylenic phosphonate 14.4.

Dibenzoyl amide 14.6 is converted into the 2-iodo compound 14.7, as described above, and coupled with a dialkyl propynyl phosphonate 14.8, (Synthesis, (1999), 2027) to yield the acetylenic phosphonate 14.9. After deprotection of the benzoyl groups, the 5,6-dihydroxy-2-methyl-pyrimidine-4-carboxylic acid (cyclopent-3-enylmethyl)-amide phosphonate compound 14.10 is obtained.

Using the above procedures, but employing, in place of the iodoamide 14.7, different iodoamides 14.2, and/or different acetylenic phosphonates 14.3, the corresponding products 14.4 are obtained.

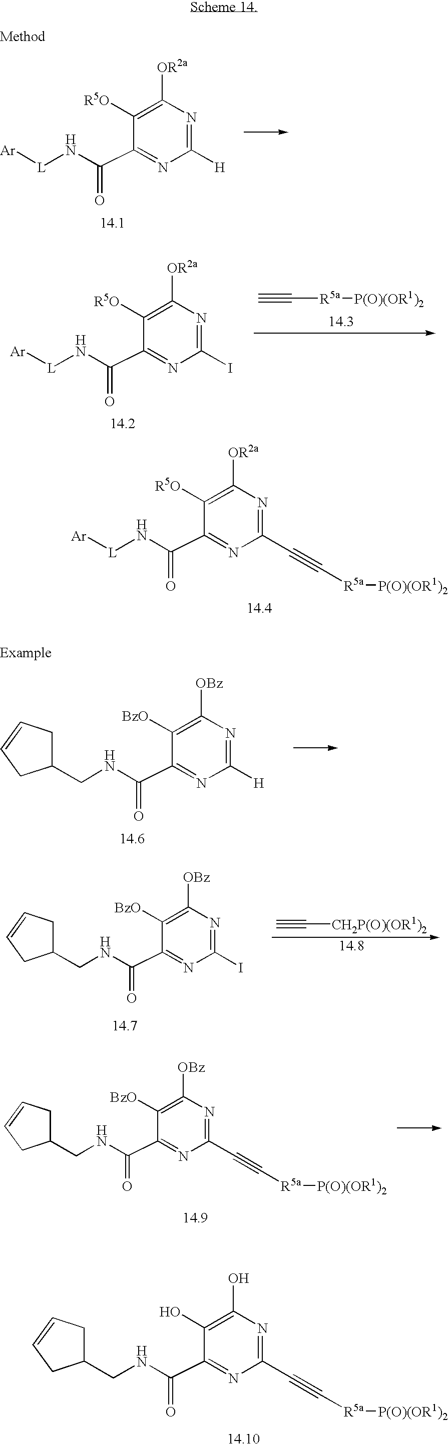

Scheme 15 depicts the preparation of phosphonates IIa in which the phosphonate is directly attached to pyrimidinone at the 2-position. In this procedure, a protected 2-bromopyrimidyl 15.1 is coupled, in the presence of a palladium catalyst, as described in Scheme 3, with a dialkyl phosphite 15.2, to give after deprotection the aryl phosphonate 15.3.

For example, 4-oxo-5-(tetrahydro-pyran-2-yloxy)-3-triisopropylsilanyl-3,4-dihydro-pyrimidine-6-carboxylic acid [1-(3-chloro-4-fluoro-phenyl)-1-methyl-ethyl]-amide 15.4, is converted, using the procedures described above, is brominated to give 2-bromo-4-oxo-5-(tetrahydro-pyran-2-yloxy)-3-triisopropylsilanyl-3,4-dihydro-pyrimidine-6-carboxylic acid [1-(3-chloro-4-fluoro-phenyl)-1-methyl-ethyl]-amide 15.5. The product is then coupled, in the presence of tetrakis(triphenylphosphine)palladium(0) and triethylamine, as described in Scheme 3, with a dialkyl phosphite 15.6 (for example, R¹=ethyl), to afford, after desilylation of the phenol, the pyrimidinone 2-phosphonate 15.7 which can be deprotected under acidic conditions to 15.8.

Using the above procedures, but employing, in place of the bromoamide 15.5, different bromoamides 15.1, the corresponding products 15.3 are obtained.

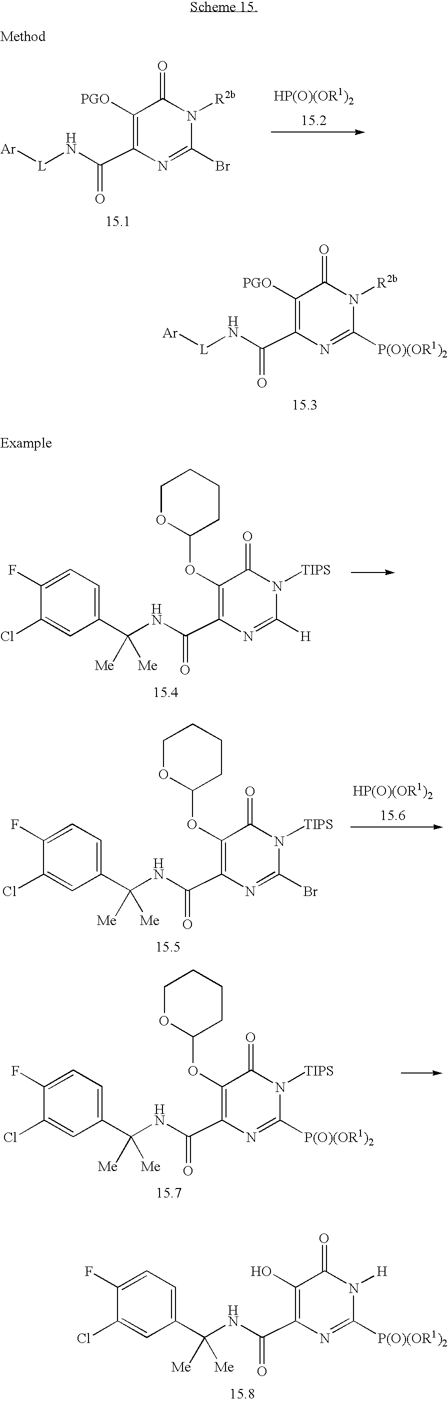

Schemes 16-18 illustrate methods for the preparation of the 2-amino linked phosphonate esters Ia and IIa.

Scheme 16 depicts the N-3 sulfonation of 2-phosphonate compounds. In this procedure, 16.1, in which the 5-hydroxyl group is protected, prepared as described in Scheme 11, is reacted with a sulfonyl chloride 16.2 or a sulfonic acid 16.3, in which R⁴¹ can be C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, or C₂-C₂₀ substituted heterocycle, to afford sulfonamide 16.4. The reaction between an amine and a sulfonyl chloride, to produce the sulfonamide, is conducted at ambient temperature in an inert solvent such as dichloromethane, in the presence of a tertiary base such as triethylamine. The reaction between a sulfonic acid and an amine to afford a sulfonamide is conducted in a polar solvent such as dimethylformamide, in the presence of a carbodiimide such as dicyclohexyl carbodiimide, for example as described in Synthesis, (1976), 339.

For example, the 5-protected phosphonate diisobutyl ester 16.5, prepared by the methods described above, is reacted in dichloromethane solution with one molar equivalent of ethylsulfonyl chloride 16.6 and triethylamine, to produce 16.7. Desilylation of 16.7 gives {2-[(4-dimethylcarbamoyl-1-ethanesulfonyl-5-hydroxy-6-oxo-1,6-dihydro-pyrimidin-2-yl)-methyl-amino]-ethyl}-phosphonic acid di-sec-butyl ester 16.8.

Using the above procedures, but employing, in place of the amine phosphonate 16.5, different phosphonates 16.1, and/or different sulfonyl chlorides 16.2 or sulfonic acids 16.3, the corresponding products 16.4 are obtained.

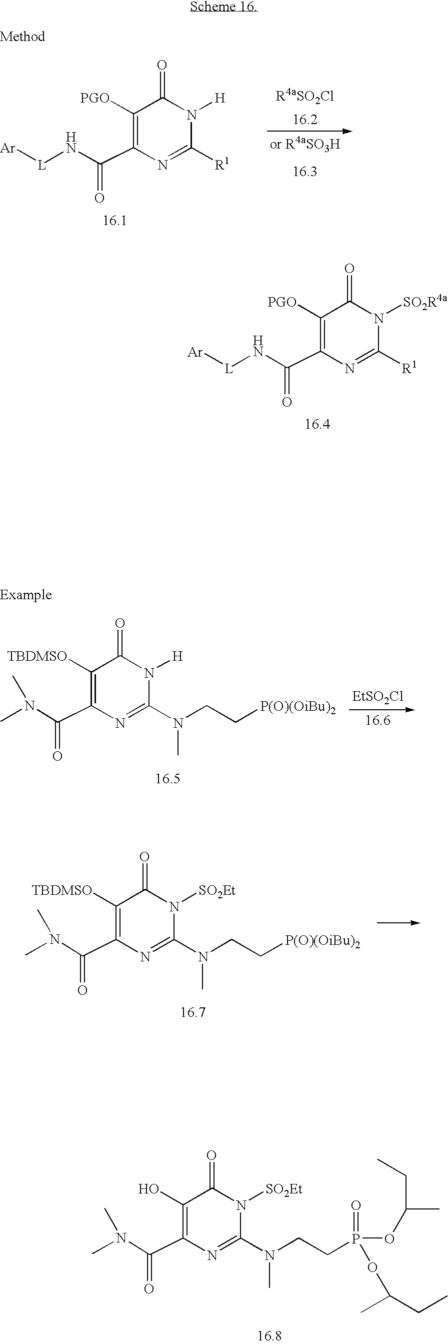

Scheme 17 depicts an alternative method for the preparation of phosphonate esters IIa in which the phosphonate group is attached by means of a variable carbon chain from a 2-sulfonamido group. In this procedure, a dialkyl amino-substituted phosphonate 17.1, in which the group R^{5s} is as defined in Scheme 4, is reacted with a sulfonyl chloride 17.2 or sulfonic acid 17.3, as described in Scheme 16, to yield the sulfonamide 17.4. The product is then reacted with a bromoamide 17.5, to prepare the displacement product 17.6. The displacement reaction is performed in a basic solvent such as pyridine or quinoline, at from about 80° to reflux temperature, optionally in the presence of a promoter such as copper oxide, as described in WO 0230930 A2 Example 154.

For example, a dialkyl 4-aminophenyl phosphonate 17.7 (Epsilon) is reacted in dichloromethane solution with one molar equivalent of methanesulfonyl chloride 17.8 and triethylamine, to give the sulfonamide 17.9. The product is then reacted in pyridine solution at reflux temperature with 2-bromo-6-(4-fluoro-benzylcarbamoyl)-3-methyl-6-benzoyloxy-3,4-dihydro-pyrimidin-5-yl ester 17.10, prepared by the methods described above, and copper oxide, to yield the sulfonamide 17.11.

Using the above procedures, but employing, in place of the amine phosphonate 17.7, different phosphonates 17.1, and/or different sulfonyl chlorides 17.2 or sulfonic acids 17.3, the corresponding products 17.6 are obtained.

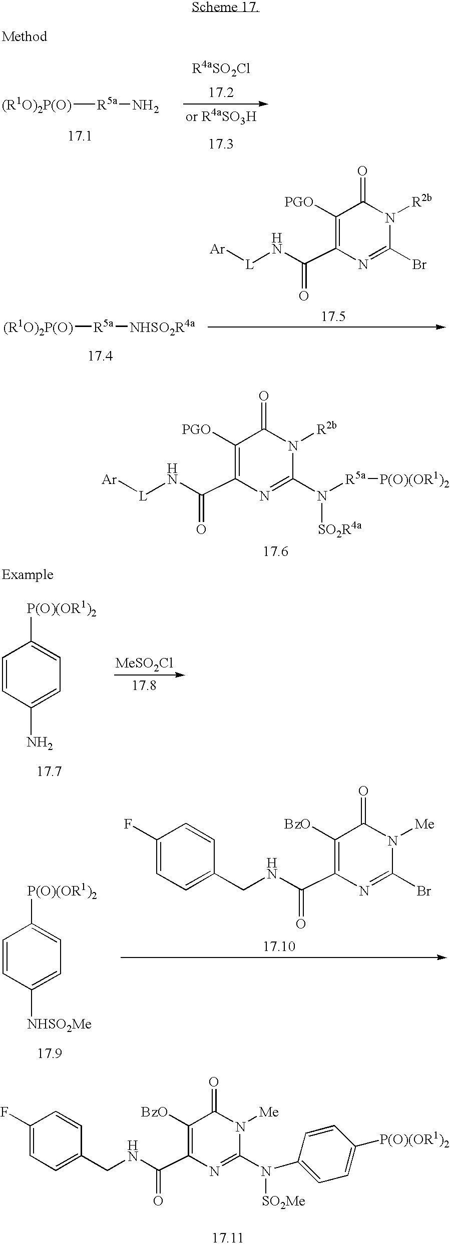

Scheme 18 depicts an alternative method for the preparation of phosphonate esters Ia in which the phosphonate group is attached by means of a variable carbon chain. In this procedure, a phenol-protected 5-bromo substituted amide 18.1 is reacted, as described in Scheme 17, with a sulfonamide 18.2, to give the displacement product 18.3. The product is then reacted with a diallyl bromoalkyl phosphonate 18.4 to afford, after deprotection of the phenol, the alkylated compound 18.5. The alkylation reaction is performed in a polar aprotic solvent such as dimethylformamide or DMPU, at from ambient temperature to about 100° C., in the presence of a base such as sodium hydride or lithium hexamethyl disilylazide.

For example, benzoic acid 2-bromo-4-hydroxy-6-[1-(3-methoxy-phenyl)-1-methyl-ethylcarbamoyl]-pyrimidin-5-yl ester 18.6, prepared by the methods described above, is reacted in pyridine solution at reflux temperature with one molar equivalent of propanesulfonamide 18.7 and copper oxide, to afford the sulfonamide 18.8. The product is then reacted in dimethylformarnmide solution with one molar equivalent of a dialkyl bromoethyl phosphonate 18.9 (Aldrich) and lithium hexamethyl disilylazide, to give after debenzoylation, the sulfonamide phosphonate 18.10. The benzoyl protecting group is removed, for example, by reaction with 1% methanolic sodium hydroxide at ambient temperature, as described in Tetrahedron, 26, 803, 1970.

Using the above procedures, but employing, in place of the bromo compound 18.6, different bromo compounds 18.1, and/or different sulfonamides 18.2, and/or different phosphonates 18.4, the corresponding products 18.5 are obtained.

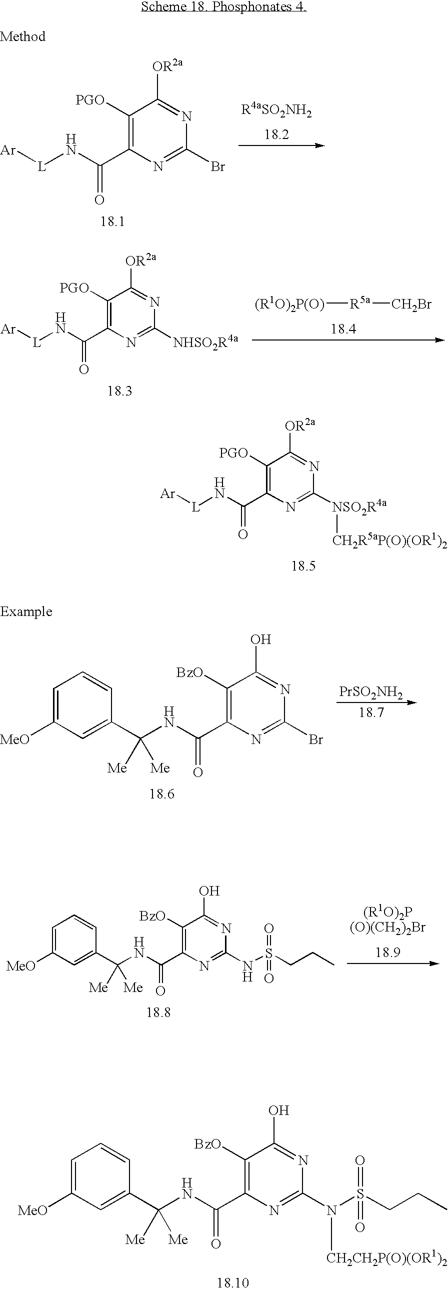

Schemes 19-21 illustrate methods for the preparation of 2-amino linked phosphonate esters Ia and IIa.

Scheme 19 illustrates the preparation of phosphonates IIa in which the phosphonate group is attached by means of a variable carbon chain. In this procedure, a bromo-substituted sulfonic acid 19.1 is subjected to an Arbuzov reaction with a trialkyl phosphite 19.2 to give the phosphonate 19.3. The Arbuzov reaction is performed by heating the bromo compound with an excess of the trialkyl phosphite at from 100° C. to 150° C., as described in Handbook of Organophosphorus Chem., 1992, 115-72. The resulting phosphonate is then reacted with an amine 19.4, either directly, in the presence of a carbodiimide, or by initial conversion to the sulfonyl chloride, as described in Scheme 16, to afford, after deprotection of the phenolic hydroxyl group, the sulfonamide 19.5.

For example, 3-bromopropanesulfonic acid 19.6 (Sigma) is heated at 130° C. with a trialkyl phosphite 19.7 to give the phosphonate 19.8. The product is then reacted in DMPU solution with 19.9, prepared by the methods described above, in the presence of dicyclohexylcarbodiimide, to give, after desilylation, by reaction with tetrabutylammonium fluoride in tetrahydrofuran, the sulfonamide 19.10.

Using the above procedures, but employing, in place of the bromo sulfonic acid 19.6, different bromosulfonic acids 19.1, and/or different amines 19.4, the corresponding products 19.5 are obtained.

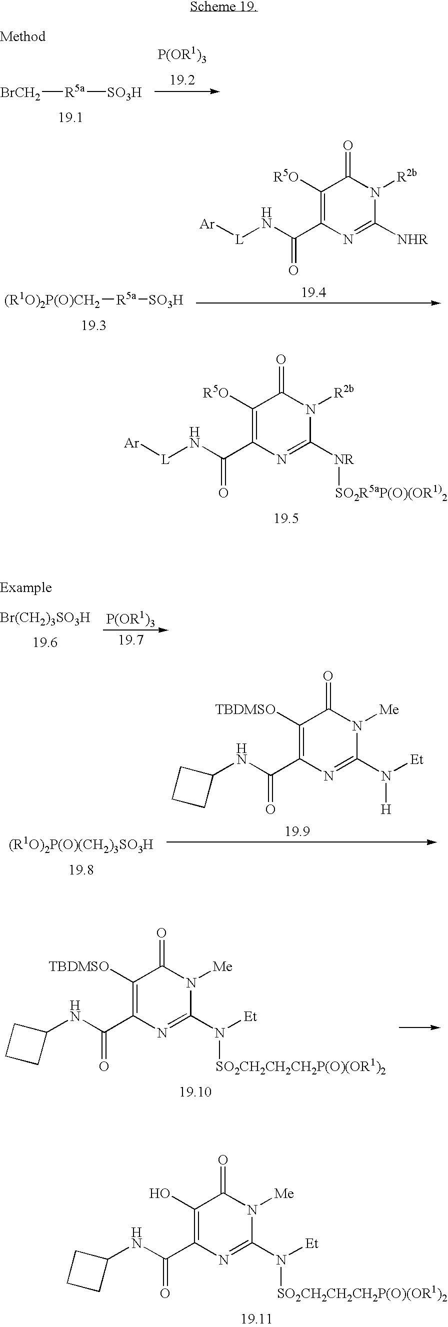

Scheme 20 illustrates the preparation of phosphonates IIa in which the phosphonate group is attached by means of a saturated or unsaturated carbon chain and an aromatic or heteroaromatic group. In this procedure, a vinyl-substituted sulfonic acid 20.1 is coupled, in a palladium-catalyzed Heck reaction, as described in Scheme 4, with a dibromoaromatic or heteroaromatic compound 20.2, to yield the sulfonic acid 20.3. The product is then coupled, in the presence of a palladium catalyst, as described in Scheme 3, with a dialkyl phosphite HP(O)(OR¹)₂, to give the phosphonate 20.4. The latter compound is then reacted, as described above, with an amine 20.5, either directly, in the presence of a carbodiimide, or by initial conversion to the sulfonyl chloride, as described in Scheme 16, to afford, after deprotection of the phenolic hydroxyl group, the sulfonamide 20.6. Optionally, the double bond is reduced, either catalytically or chemically, as described in Scheme 4, to afford the saturated analog 20.7.

For example, vinylsulfonic acid 20.8 (Sigma) is coupled, in dioxane solution, in the presence of tetrakis(triphenylphosphine)palladium (0) and potassium carbonate, with 2,5-dibromothiophene 20.9, to form the coupled product 20.10. The product is then reacted in toluene solution at 100° C. with a dialkyl phosphite 20.11, triethylamine and a catalytic amount of tetrakis(triphenylphosphine)palladium (0), to produce the phosphonate 20.12. This material is then reacted, in dimethylformamide solution at ambient temperature, as described above, with 4-fluoro-benzylamide 20.13, prepared by the methods described above, in the presence of dicyclohexylcarbodiimide, to give, after desilylation, using tetrabutylammonium fluoride, the sulfonamide 20.14. Hydrogenation of the double bond, for example using 5% palladium on carbon as catalyst, then yields the saturated analog 20.15.

Using the above procedures, but employing, in place of the sulfonic acid 20.8, different sulfonic acids 20.1, and/or different dibromoaromatic compounds 20.2, and/or different amines 20.5, the corresponding products 20.6 and 20.7 are obtained.

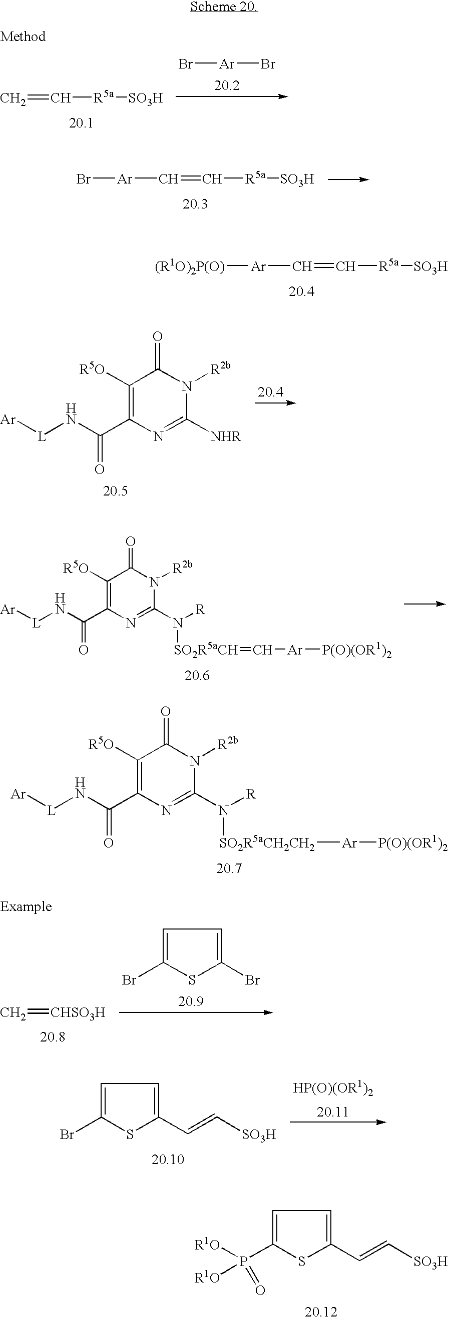

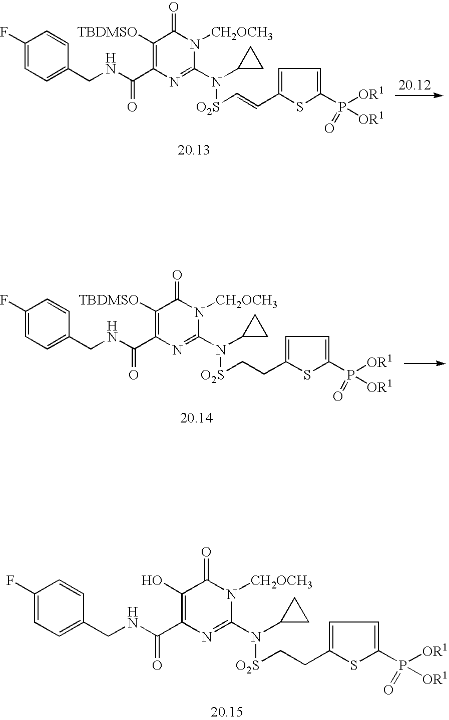

Scheme 21 illustrates the preparation of phosphonates Ia in which the phosphonate group is attached by means of a variable carbon chain. In this procedure, an aliphatic bromo-substituted sulfonic acid 21.1 is subjected to an Arbuzov reaction with a trialkyl phosphite, as described in Scheme 19, to give the phosphonate 21.2. Alternatively, an aryl bromosulfonic acid 21.1 is coupled, as described in Scheme 3, with a dialkyl phosphite, to give the phosphonate 21.2. The product is then reacted with an amine 21.3 to afford the sulfonamide 21.4. The latter compound is then reacted, as described in Scheme 17, with a bromoamide 21.5, to give the displacement product 21.6.

For example, 4-bromobenzenesulfonic acid 21.7 is reacted, as described in Scheme 20, with a dialkyl phosphite to form the phosphonate 21.8. The product is then reacted with phosphoryl chloride to afford the corresponding sulfonyl chloride, and the latter compound is reacted, in dichloromethane solution, in the presence of triethylamine, with 2-methoxyethylamine 21.9, to yield the sulfonamide 21.10. This material is then reacted, in pyridine solution at reflux temperature, with 2-bromo-4,5-dimethoxy-pyrimidine-6-carboxylic acid 4-fluoro-benzylamide 21.11, prepared by the methods described above, and copper oxide, to give the 2-sulfonamide phosphonate 21.12.

Using the above procedures, but employing, in place of the sulfonic acid 21.7, different sulfonic acids 21.1, and/or different amines 21.3, and/or different bromo compounds 21.5, the corresponding products 21.6 are obtained.

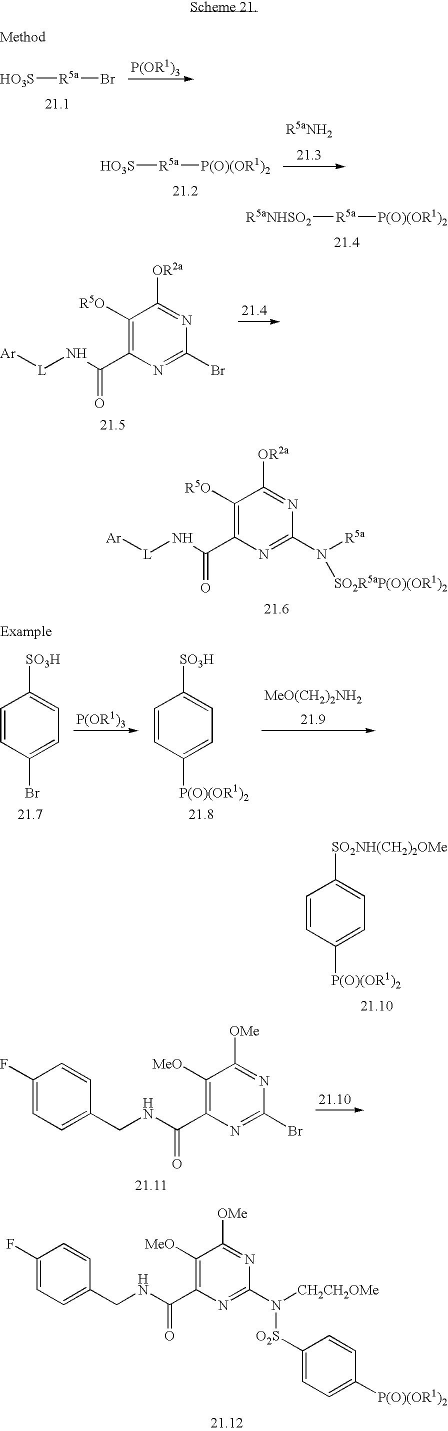

### Preparation of Phosphonate Esters Ia and IIa.

Scheme 22 depicts the preparation of phosphonate esters Ia in which the phosphonate group is attached by means of an cyclic sulfonamide group at the 2-amino position. In this procedure, a cyclic sulfonamide 22.1, where m and n are independently 1, 2, 3, 4, 5, or 6, and incorporating a secondary amine, is coupled, as described in Scheme 5, with a dialkyl carboxy-substituted phosphonate 22.2 to produce the amide 22.3. The product is then reacted with a bromoamide 22.4 to afford the displacement product 22.5.

Alternatively, the cyclic sulfonamide 22.1 is protected to give the analog 22.6. Sulfonamides are protected, for example, by conversion into the N-acyloxymethyl derivatives, such as the pivalyloxymethyl derivative or the benzoyloxymethyl derivative, by reaction with the corresponding acyloxymethyl chloride in the presence of dimethylaminopyridine, as described in Bioorg. Med. Chem. Lett., 1995, 5, 937, or by conversion into the carbamate derivative, for example the tert butyl carbamate, by reaction with an alkyl, aryl or aralkyl chloroformate, in the presence of a base such as triethylamine, as described in Tet. Lett., 1994, 35, 379. The protected sulfonamide is reacted with a dialkyl bromoalkyl phosphonate 22.7 to form the alkylated product 22.8. The alkylation reaction is effected as described in Scheme 8. The product is then deprotected to yield the sulfonamide 22.9. Deprotection of pivalyloxymethyl amides is effected by treatment with trifluoroacetic acid; deprotection of benzyloxymethyl amides is effected by catalytic hydrogenation, as described in Protective Groups in Organic Synthesis, by T. W. Greene and P. G. M. Wuts, Wiley, Second Edition 1990, p. 398. Sulfonamide carbamates, for example the tert butyl carbamate, are deprotected by treatment with trifluoroacetic acid. The sulfonamide 22.9 is then reacted with the bromoamide 22.10 to give the displacement product 22.11.

For example, [1,2,5]thiadiazepane 1,1-dioxide 22.11A (WO 0230930A2 p. 321) is reacted in dioxane solution with equimolar amounts of a dialkyl 3-carboxypropyl phosphonate 23.12, (Epsilon) and dicyclohexylcarbodiimide, to produce the amide 22.13. This material is reacted in pyridine solution at reflux temperature with 2-bromo-3-methyl-4-oxo-5-triisopropylsilanyloxy-3,4-dihydro-pyrimidine-6-carboxylic acid 4-fluoro-benzylamide 22.14, prepared by the methods described above, and copper oxide, to afford the displacement product 22.15.

As a further example, the sulfonamide 22.11A is reacted in dichloromethane with one molar equivalent of t-Boc anhydride, triethylamine and dimethylaminopyridine, to give 1,1-dioxo-[1,2,5]thiadiazepane-2-carboxylic acid tert-butyl ester 22.16. The product is then reacted at ambient temperature in dimethylformamide solution with a dialkyl 4-bromomethyl benzyl phosphonate 22.17, (Tetrahedron, 1998, 54, 9341) and potassium carbonate, to yield the alkylation product 22.18. The BOC group is removed by treatment with trifluoroacetic acid to give the sulfonamide 22.19, and this material is reacted, as described above, with 2-bromo-3,4-dihydroxy-pyrimidine-6-carboxylic acid 3-fluoro-benzylamide 22.20, prepared by the methods described above, to afford the displacement product 22.21.

Using the above procedures, but employing, in place of the sulfonamide 22.11A, different sulfonamides 22.1, and/or different carboxylic acids 22.2 or alkyl bromides 22.7, and/or different bromides 22.4, the corresponding products 22.5 and 22.11 are obtained.

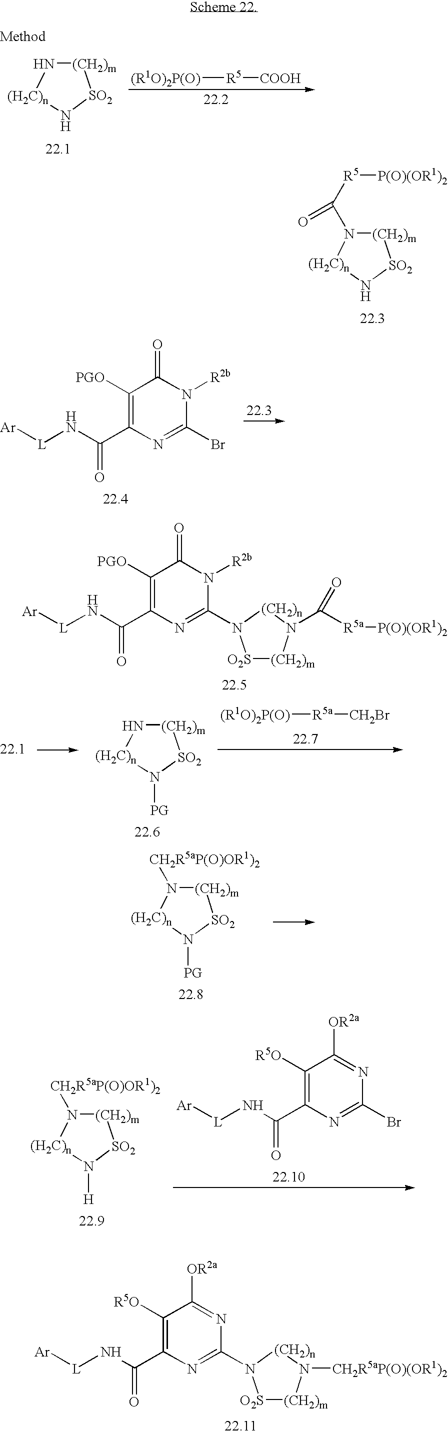

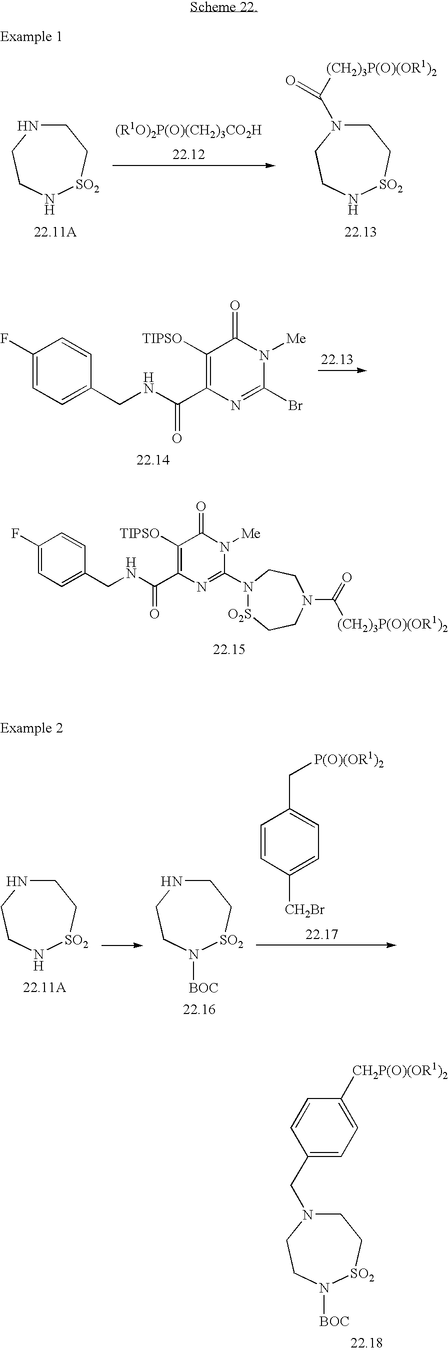

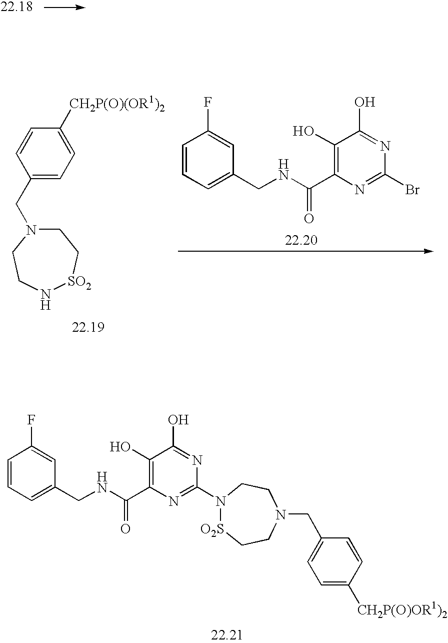

Scheme 23 depicts the preparation of phosphonates IIa in which the phosphonate group is attached by means of an aryl or heterocycle group. In this procedure, a bromoaryl-substituted cyclic sulfonamide, prepared as described in J. Org. Chem., (1991), 56, 3549, from the corresponding bromoaryl or bromoheterocycle acetic acid and a vinyl sulfonic ester, is coupled, as described in Scheme 3, with a dialkyl phosphite to afford the phosphonate 23.2. The product is then reacted, as described above, with a bromoamide 23.3 to yield the displacement product 23.4.

For example, 4-(4-bromo-phenyl)-[1,2]thiazinane 1,1-dioxide 23.5 (J. Org. Chem., 1991, 56:3549) is reacted in dimethylformamide solution with a dialkyl phosphite 23.6 and tetrakis(triphenylphosphine)palladium(0), to give the phosphonate 23.7. The product is then reacted with 2-bromo-3-(2-methoxy-ethyl)-4-oxo-5-triisopropylsilanyloxy-3,4-dihydro-pyrimidine-6-carboxylic acid (5-fluoro-indan-1-yl)-amide 23.8, prepared by the methods described above, to give the phosphonate 23.9.

Using the above procedures, bit employing, in place of the sulfonamide 23.5, different sulfonamides 23.1, and/or different bromo compounds 23.3, the corresponding products 23.4 are obtained.

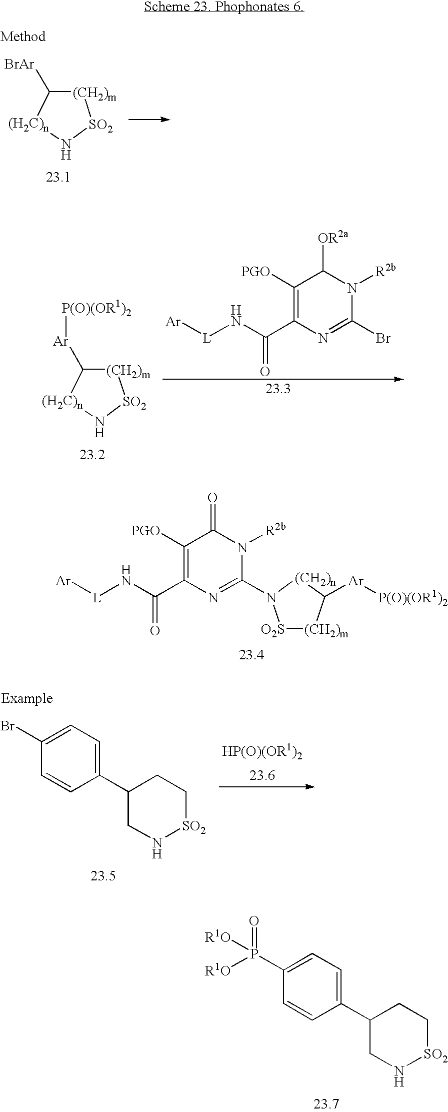

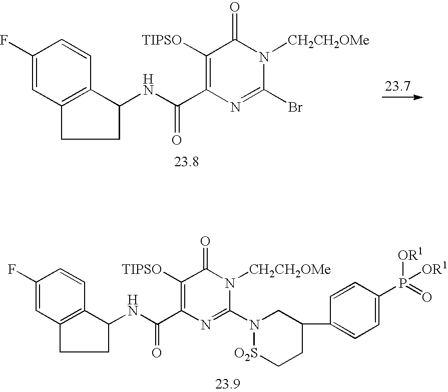

Scheme 24 depicts the preparation of phosphonates Ia in which the phosphonate group is attached by means of an amide linkage. In this procedure, a carboxy-substituted cyclic sulfonamide 24.1 is coupled with an amino-substituted dialkyl phosphonate 24.2, as described in Scheme 5, to give the amide 24.3. The product is then reacted with the bromoamide 24.4 to afford the displacement product 24.5.

For example, 1,1-dioxo-[1,2]thiazinane-3-carboxylic acid 24.6 (Izvest. Acad. Nauk. SSSR Ser. Khim., 1964, 9, 1615) is reacted in dimethylformamide solution with equimolar amounts of an amino-substituted butyl phosphonate 24.7 (Acros) and dicyclohexylcarbodiimide, to afford the amide 24.8. The latter compound is then condensed with 2-bromo-5,6,7,8,8a,10a-hexahydro-9,10-dioxa-1,3-diaza-anthracene-6-carboxylic acid [1-(3-chloro-4-fluoro-phenyl)-ethyl]-amide 24.9, prepared by the methods described above, to give the product 24.10.

Using the above procedures, but employing, in place of the sulfonamide 24.6, different sulfonamides 24.1, and/or different bromo compounds 24.4, the corresponding products 24.5 are obtained.

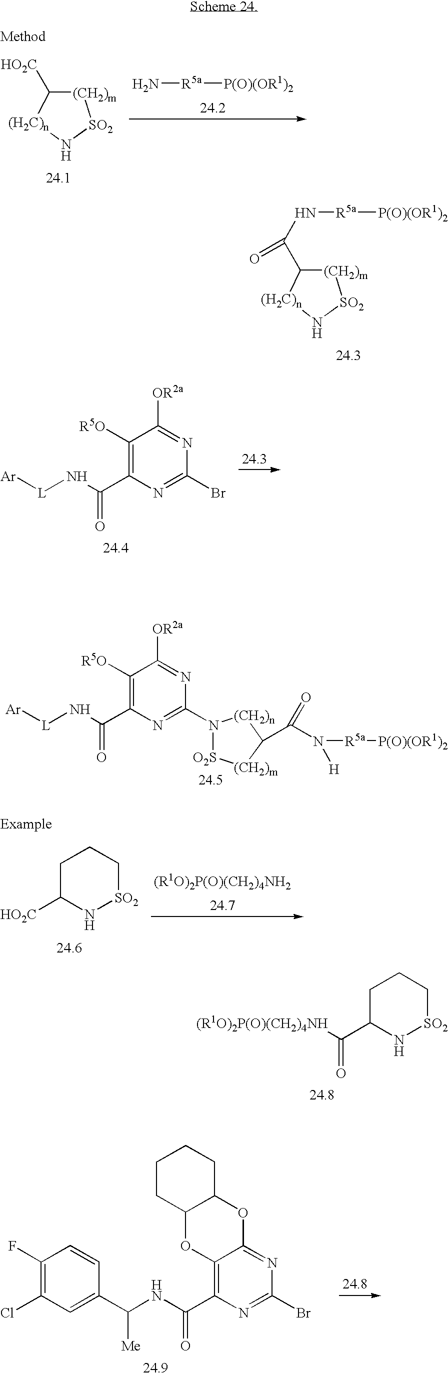

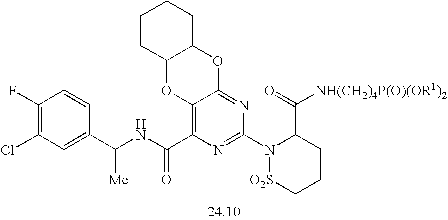

Schemes 25-27 illustrate methods for the preparation of the phosphonate esters Ia and IIa in which the phosphonate is attached by means of a carbon link or a variable carbon chain incorporating a heteroatom. In these procedures, for example, a tolyl-substituted pyrimidine 25.1 is reacted with a free radical brominating agent such as N-bromosuccinimide to prepare the bromomethyl derivative 25.3. The benzylic bromination reaction is performed at reflux temperature in an inert organic solvent such as hexachloroethane or ethyl acetate, optionally in the presence of an initiator such as dibenzoyl peroxide. The bromomethyl compound 25.3 is then reacted with a trialkyl phosphite in an Arbuzov reaction, as described in Scheme 19, to give, after deprotection of the phenolic hydroxyl group, the phosphonate 25.4.

Alternatively, the benzylic bromide 25.3 is reacted with a dialkyl hydroxy, mercapto or amino-substituted phosphonate 25.5, to afford, after deprotection of the phenolic hydroxyl group, the displacement product 25.6. The displacement reaction is effected at from ambient temperature to about 100° C., in a polar organic solvent such as dimethylformamide or DMPU, in the presence of a suitable base such as sodium hydride or lithium hexamethyldisilazide, for instances in which Y is O, or cesium carbonate or triethylamine for instances in which Y is S or N.

For example 6-p-tolyl-2,3,3a,9a-tetrahydro-1H-4,9-dioxa-5,7-diaza-cyclopenta[b]naphthalene-8-carboxylic acid 4-fluoro-benzylamide 25.8 is reacted with one molar equivalent of N-bromosuccinimide in ethyl acetate at reflux, to afford the bromomethyl analog 25.9. This product is reacted with a dialkyl hydroxyethyl phosphonate 25.11 (Epsilon) and sodium hydride in dimethylformamide at 80° C., to yield, after desilylation, the phosphonate 25.12. Alternatively, the bromomethyl compound 25.9 is reacted at 120° C. with a trialkyl phosphite, to obtain, after desilylation, the phosphonate 25.10.

Using the above procedures, but employing, in place of the anhydride 25.7, different anhydrides 25.1, and/or different phosphonates 25.5, the corresponding products 25.4 and 25.6 are obtained.

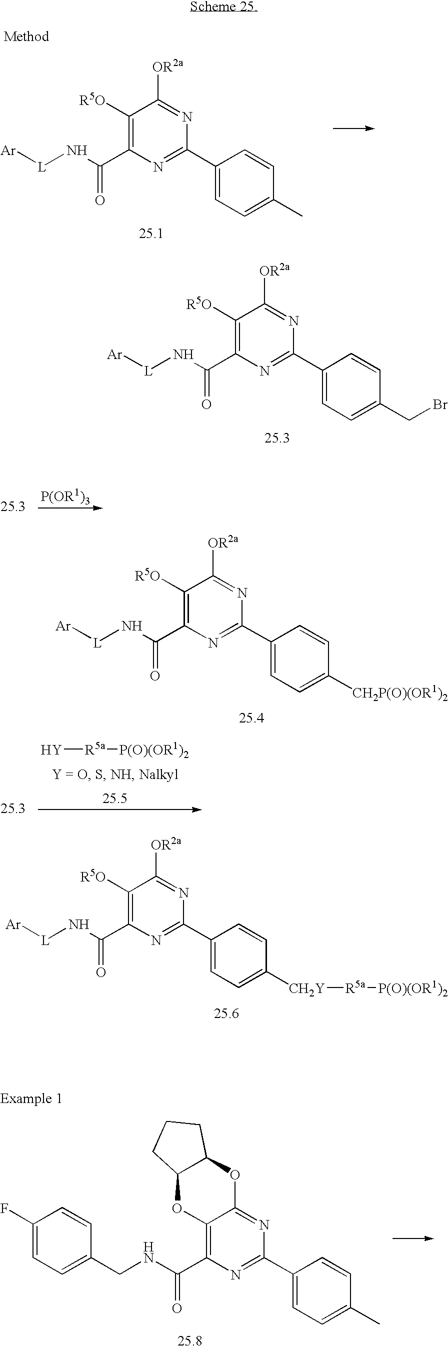

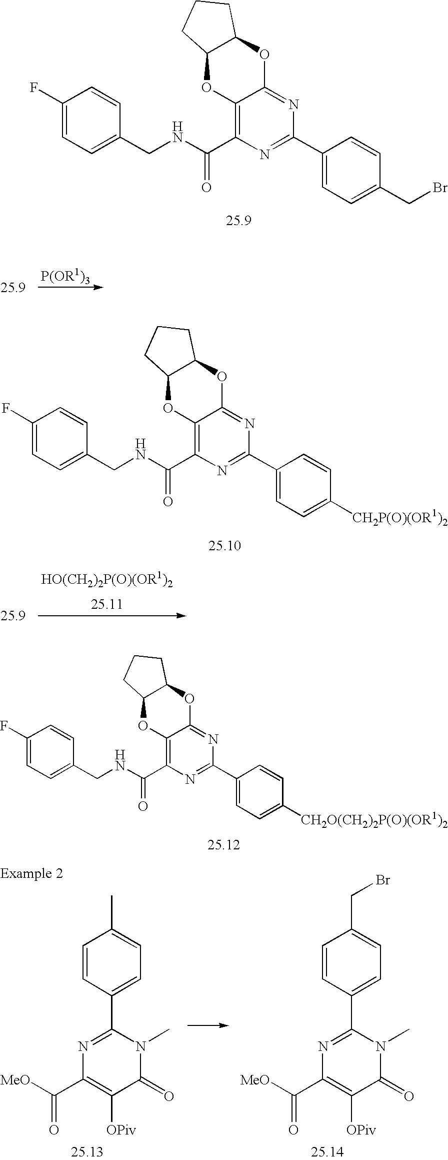

Into a flask containing 25.13 (60 mg, 0.168 mmol, 1 equiv.) was dissolved CCl₄ (3.5 ml) and benzoyl peroxide (4 mg, 0.017 mmol, 0.1 equiv.) before N-Bromosuccinimide was added. Reaction was refluxed for 4 hr, cooled and concentrated in vacuo. Silica gel chromatography was carried out using Hexanes/Ethyl Acetate 7/3 to furnish 47 mg of pyrimidinone 25.14 (65%, 0.0109 mmol).

¹H NMR (300 MHz) CDCl₃ ∂: 7.53 (s, 4H), 4.52 (s, 2H), 3.91 (s, 3H), 3.51 (s, 3H), 1.42 (s, 9H).

R_{f}: 0.2 Hexanes/Ethyl Acetate (7/3).

MS: 437.16 (M+1), 439.16 (M+3).

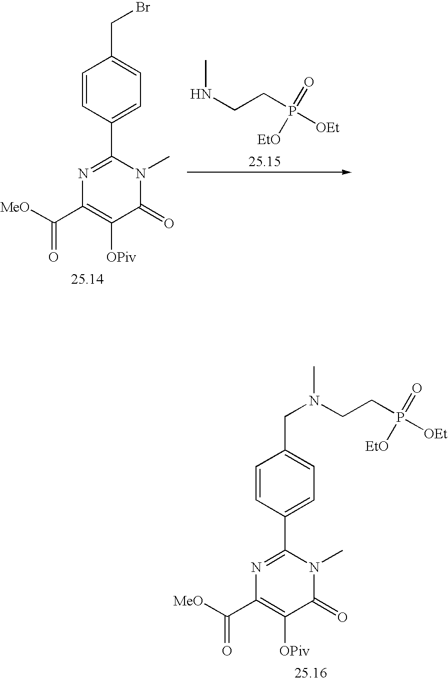

280 mg (0.64 mmol, 1 equiv) of bromide 25.14 was dissolved in THF (6 ml, 0.1 M) and to it added the amine phosphate 25.15, [diethyl 2-(methylamino)ethylphosphonate] and heated to 50° C. for 12 hr. Mixture was concentrated in vacuo and purified by silica gel flash chromatography using Ethyl Acetate/Methanol 4/1 to obtain 190 mg of phosphonate 25.16 (54%, 0.34 mmol).

¹H NMR (300 MHz) CDCl₃∂: 7.49 (s, 4H), 4.14-4.12 (s, 4H), 3.91 (s, 3H), 3.58 (s, 2H), 3.49 (s, 3H), 2.78-2.75 (s, 2H), 2.21 (s, 3H), 2.15-1.95 (m, 2H), 1.42 (s, 9H), 1.33 (t, J=7.2 Hz, 6H).

R_{f}: 0.2 Hexanes/Ethyl Acetate (7/3).

MS: 552.27 (M+1).

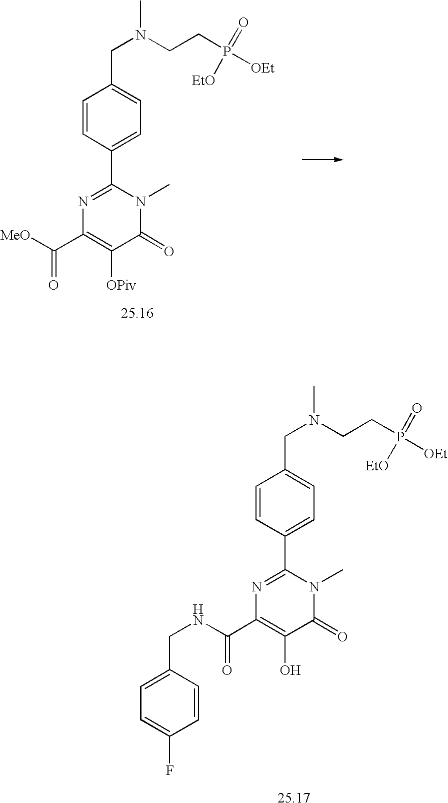

100 mg (0.18 mmol, 1 equiv.) of amine 25.16 was dissolved in anhydrous acetonitrile (5 ml, 0.68 M) in a microwave vial and to it placed p-Fluorobenzylamine (104 μl, 0.91 mmol, 5 equiv) and capped. It was then placed in a microwave and heated to 80° C. for 1 hr. The reaction was then concentrated in vacuo and the reaction mixture was purified in HPLC to obtain the pyrimidinone 25.17 (70 mg, 0.098 mmol; 68%).

¹H NMR (300 MHz) DMSO 12.48 (s, 1H), 9.33 (s, 1H), 7.77 (d, J=8.4 Hz, 2H), 7.74 (d, J=8.4 Hz, 2H), 7.37-7.32 (m, 2H), 7.17-7.11 (m, 2H), 4.52-4.35 (m, 2H), 4.45 (d, 6.3 Hz, 2H), 4.03 (q, J=7.2 Hz, 4H), 3.55-3.51 (m 2H), 3.30 (s, 3H), 2.72 (s, 3H), 2.43-2.21 (m, 2H).

¹⁹F NMR (300 MHz) DMSO ∂: −74.25

MS: 561.31 (M+1).

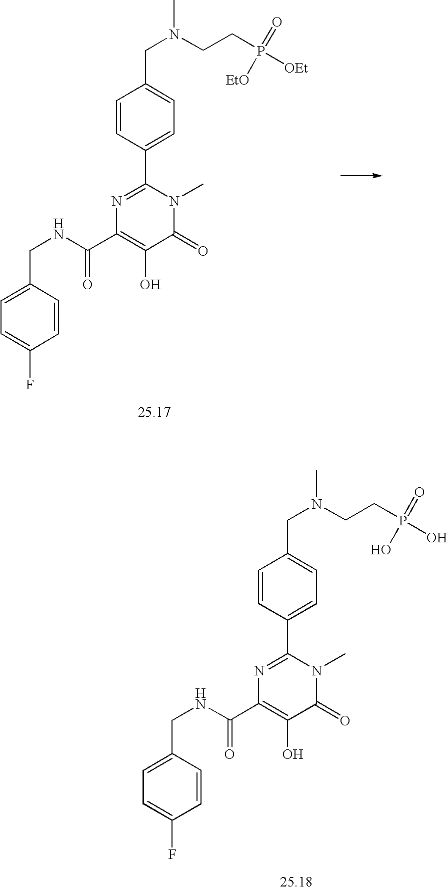

35 mg (0.062 mmol, 1 equiv.) of amine 25.17 was dissolved in anhydrous methylene chloride (3 ml) in a microwave vial and to it placed 2,6-lutidine (290 μl, 2.49 mmol, 40 equiv.) and TMSBr (160 μl, 1.24 mmol, 20 equiv.) and capped. It was then placed in a microwave and heated to 10° C. for 2 hr. The reaction was then concentrated in vacuo and the reaction mixture was purified in HPLC to obtain the pyrimidinone 25.18 (27 mg, 0.054 mmol, 86%).

¹H NMR (300 MHz) DMSO 12.48 (s, 1H), 9.37 (t, J=2.5 Hz, 1 N—H), 7.77 (d, J=8.4 Hz, 2H), 7.74 (d, J=8.4 Hz, 2H), 7.37-7.32 (m, 2H), 7.17-7.11 (m, 2H), 4.52-4.35 (m, 2H), 4.45 (d, 6.0 Hz, 2H), 3.30 (s, 3H), 3.35-3.21 (m, 2H), 2.72 (s, 3H), 2.13-2.02 (m, 2H).

¹⁹F NMR (300 MHz) DMSO ∂: −74.15.

³¹P NMR (300 MHz) DMSO ∂: 19.94

MS: 505.29 (M+1).

Scheme 26 illustrates the preparation of phosphonate esters IIa in which the phosphonate is attached by means of an aminomethyl linkage through the 2-position. In this procedure, a bromomethyl-substituted bicyclic amide 26.1a, prepared as described in Scheme 25, is oxidized to the corresponding aldehyde 26.1. The oxidation of halomethyl compounds to aldehydes is described, for example, in Comprehensive Organic Transformations, by R. C. Larock, VCH, 1989, p. 599ff. The transformation is effected by treatment with dimethylsulfoxide and base, optionally in the presence of a silver salt, or by reaction with trimethylamine N-oxide or hexamethylene tetramine. The aldehyde 26.1 is then reacted with a dialkyl amino-substituted phosphonate 26.2 in a reductive amination reaction (H-=reducing agent), as described in Scheme 9, to yield, after deprotection of the phenolic hydroxyl group, the aminomethyl product 26.3.

For example, 5-benzyloxymethoxy-2-(4-bromomethyl-phenyl)-4-oxo-3,4-dihydro-pyrimidine-6-carboxylic acid 3,5-dichloro-benzylamide 26.4, prepared from the anhydride 25.7, using the methods described in Scheme 25, is reacted with dimethylsulfoxide and 2,4,6-collidine at 90°, as described in J. Org. Chem., 51, 1264, 1986, to afford the aldehyde 26.5. The product is then reacted with one molar equivalent of a dialkyl aminoethyl phosphonate 26.6 (Epsilon) and sodium triacetoxyborohydride to produce, after desilylation, the phosphonate 26.7.

Using the above procedures, but employing, in place of the bromomethyl compound 26.4, different bromomethyl compounds 25.3, and/or different phosphonates 26.2, the corresponding products 26.3 are obtained.

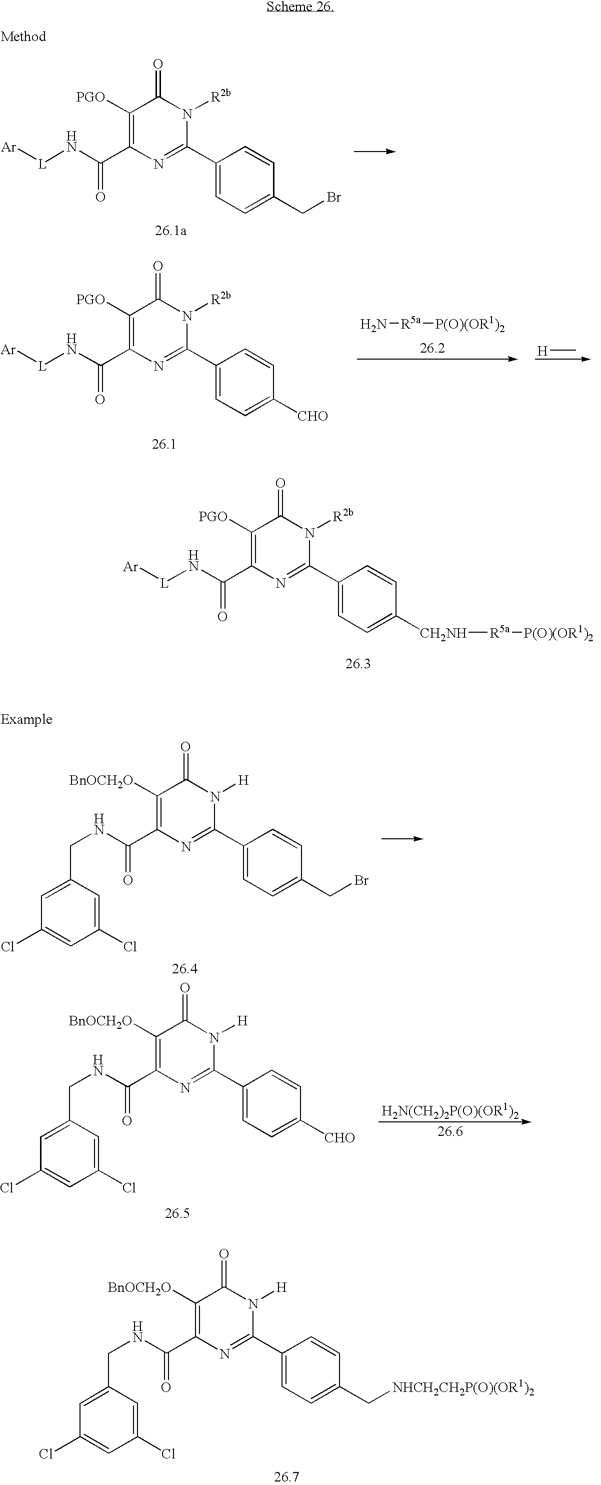

A reductive amination procedure can also be employed to attach a phosphonate ester through an amino linker. 1-Methyl-6-oxo-2-(2-oxo-ethyl)-5-triisopropylsilanyloxy-1,6-dihydro-pyrimidine-4-carboxylic acid 4-fluoro-benzylamide 26.8, prepared by the method of WO 03/03577 at page 96 can be reductively aminated by amino phosphonate reagents, 26.9, 26.10, and 26.11 to give 26.12, 26.13, and 26.14, respectively, after desilylation with tetrabutylammonium fluoride (TBAF) (Scheme 26a). As with the previous examples herein, R¹ may be further converted to other phosphorus substituents, e.g. X and Y. Embodiments of phosphonate substituent X include OPh, OAr, OCH₂CF₃, and NHR, where R is the residue of an amino acid. Embodiments of phosphonate substituent Y include a lactate ester or a phosphonamidate.

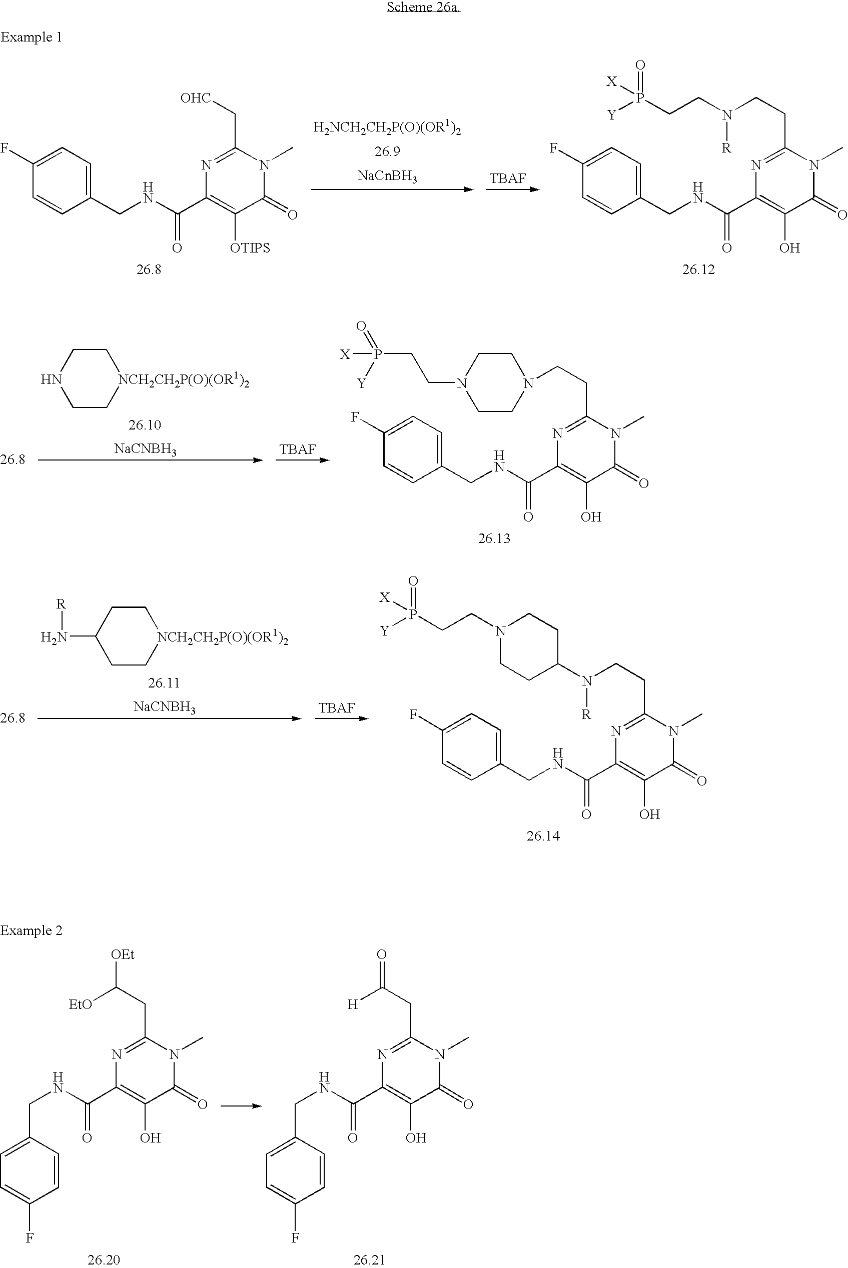

62 mg (0.16 mmol, 1 equiv.) of amine 26.20 was dissolved in anhydrous THF (4 ml) in a microwave vial and to it placed HCl (aq) (0.5 ml, 10%) and capped. It was then placed in a microwave and heated to 55° C. for 2 hr. The reaction was then concentrated in vacuo thoroughly and used in the next reaction as a crude mixture of 26.21.

MS: 352.02 (M+MeOH).

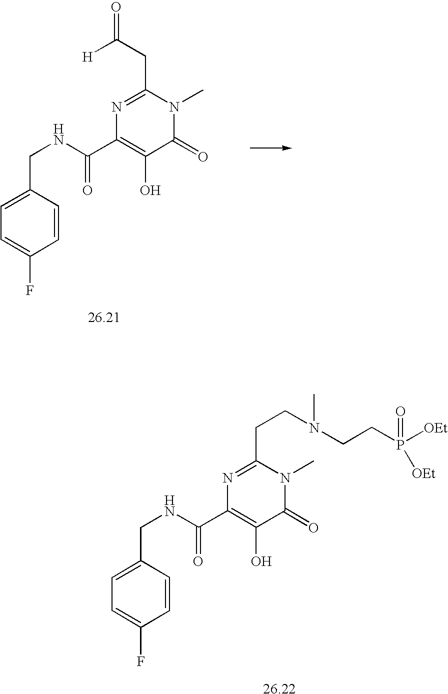

To aldehyde 26.21 was added methanol (5 ml) followed by amine 25.15 [diethyl 2-(methylamino)ethylphosphonate] (123 mg, 0.63 mmol, 14 equiv.) To this was added acetic acid (300 μl) and NaCNBH₃ (30 mg, 0.47 mmol, 3 equiv.) and the reaction was allowed to stir for 16 hr. The reaction mixture was then concentrated in vacuo, filtered and HPLC purified to furnish phosphonate 26.22 (7 mg, 0.014 mmol).

¹H NMR (300 MHz) CD₃OD 7.39-7.35 (m, 2H), 7.09-7.04 (m, 2H), 4.59 (s, 2H), 4.17-4.13 (m, 4H), 3.65 (s, 3H), 3.66-3.63 (m, 2H), 3.32-3.29 (m, 2H), 3.00 (s, 3H), 2.51-2.50 (m, 2H), 1.34 (t, J=6.6 Hz, 6H).

¹⁹F NMR (300 MHz) CD₃OD∂: −77.66.

³¹P NMR (300 MHz) CD₃OD ∂: 26.18

MS: 499.15 (M+1).

6-Oxo-1-(2-oxo-ethyl)-5-triisopropylsilanyloxy-1,6-dihydro-pyrimidine-4-carboxylic acid 4-fluoro-benzylamide 26.15, prepared from 1-allyl-5-(2,2-dimethyl-propionyloxy)-6-oxo-1,6-dihydro-pyrimidine-4-carboxylic acid methyl ester 26.16 (piv=pivalate, (CH₃)₃CC(O)—) by the method of WO 03/03577 at page 10 can be reductively aminated by amino phosphonate reagents, 26.9, 26.10, and 26.11 to give 26.17, 26.18, and 26.19, respectively after desilylation with TBAF (Scheme 26b).

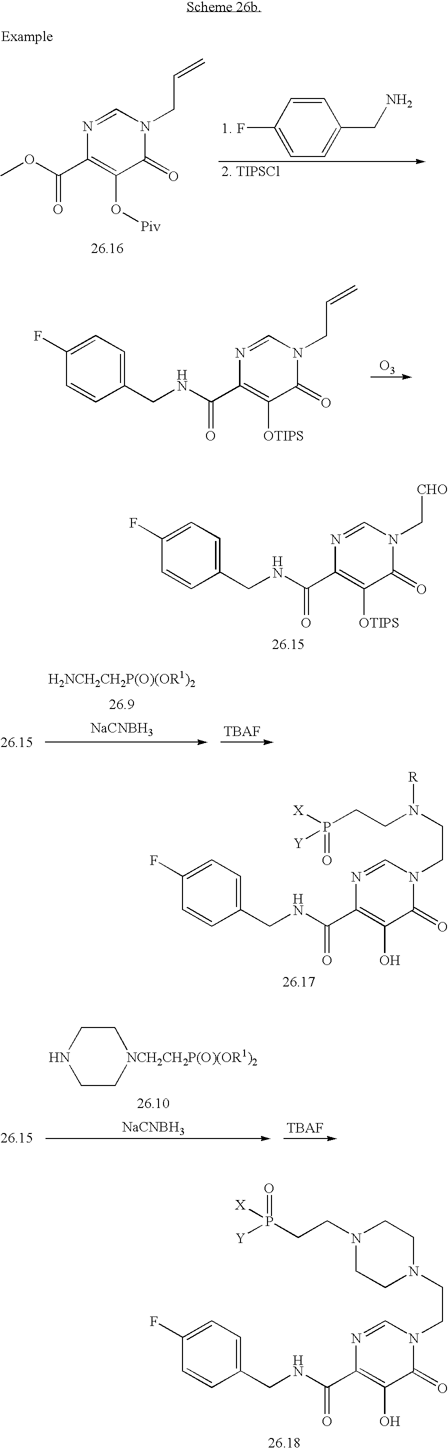

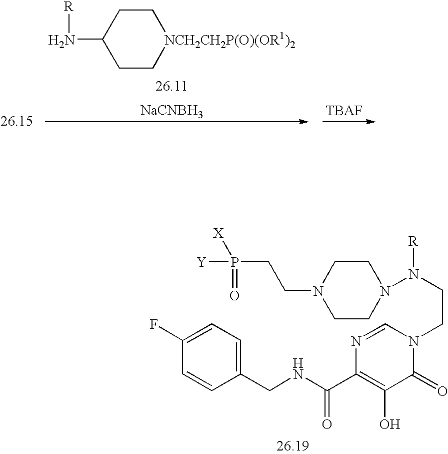

Scheme 27 illustrates the preparation of phosphonate esters Ia in which the phosphonate is attached by coupling a carboxylic acid with an amino phosphonate reagent to form an amide linkage. In this procedure, an aldehyde 27.1, or 26.1 from Scheme 26, is oxidized to the corresponding carboxylic acid 27.2. The conversion of aldehydes to the corresponding carboxylic acids is described in Comprehensive Organic Transformations, by R. C. Larock, VCH, 1989, p. 838. The reaction is effected by the use of various oxidizing agents such as, for example, potassium permanganate, ruthenium tetroxide, silver oxide or sodium chlorite. The resultant carboxylic acid 27.2 is then coupled, as described in Scheme 5, with a dialkyl amino-substituted phosphonate 27.3, to yield the amide 27.4.

For example, 2-(4-formyl-phenyl)-4-methoxy-5-triisopropylsilanyloxy-pyrimidine-6-carboxylic acid (cyclohex-3-enylmethyl)-amide 27.5 is reacted with silver oxide in aqueous sodium hydroxide, as described in Org. Syn. Coll. Vol. 4, 919, 1963, to afford the carboxylic acid 27.6. The latter compound is then reacted in dioxane solution at ambient temperature with equimolar amounts of a dialkyl aminomethyl phosphonate 27.7 (Interchim) and dicyclohexylcarbodiimide, to give, after desilylation, the amide phosphonate 27.8.

Using the above procedures, but employing, in place of the aldehyde 27.5, different aldehydes 26.1, and/or different phosphonates 27.3, the corresponding amides 27.4 are obtained. For example, 5,6-dihydroxy-pyrimidine-2,4-dicarboxylic acid 4-methyl ester 27.9, prepared by the method of WO 03/035077, p. 85, may be converted to the 4-fluorobenzyl amide 27.10 with 4-fluorobenzylamine (Scheme 27a), and the carboxylic acid group coupled with a plethora of amines, including 26.9, 26.10, and 26.11 to give 27.11, 27.12, and 27.13, respectively (Scheme 27b).

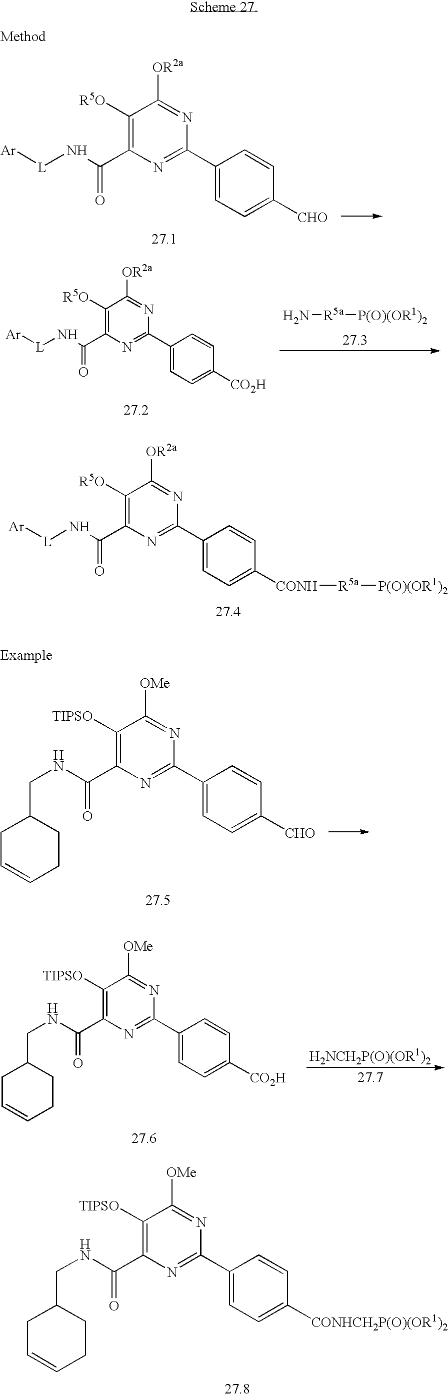

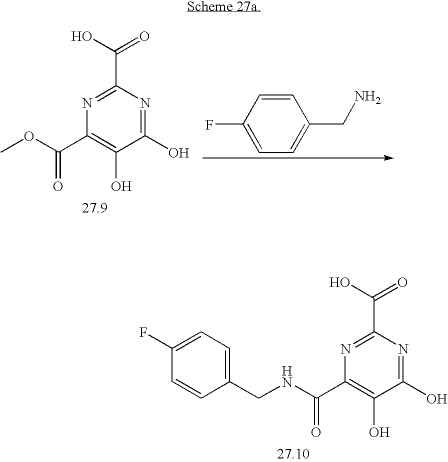

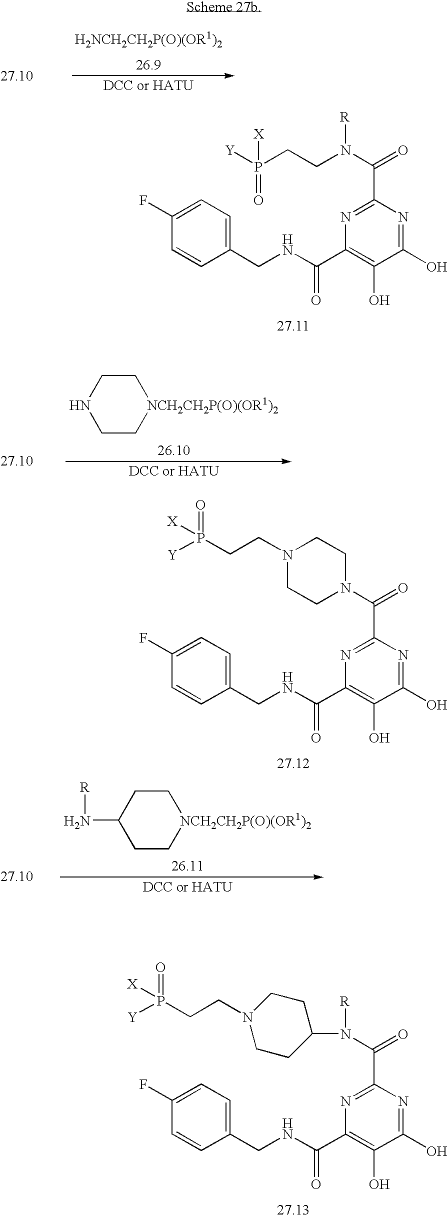

Scheme 28 illustrates the preparation of phosphonate esters Ib in which the phosphonate is attached by means of a heteroatom O or S and a variable carbon link at the 4-position. In this procedure, the 5-hydroxyl protected methyl ester 28.1 is subjected to a Mitsunobu reaction, as described in Scheme 7, with a dialkyl hydroxy or mercapto-substituted phosphonate 28.8, to produce the ether or thioether phosphonate 28.9. This compound is then reacted, as described in Scheme 3, with the amine ArLNR³H, to give amide 28.10. Alternatively, 28.1 is reacted with a dialkyl bromoalkyl-substituted phosphonate 28.5, as described in Scheme 6, to yield the ether 28.6. The latter compound is then transformed, as described above, into the amide 28.7.

In other embodiments, Scheme 28a shows 5-hydroxy-3-methyl-4-oxo-2-p-tolyl-1,6-dihydro-pyrimidine-6-carboxylic acid benzylamide 28.11 reacting with a dialkyl 2-mercaptoethyl phosphonate 28.18 (Zh. Obschei. Khim., (1973), 43, 2364), diethylazodicarboxylate and triphenylphosphine to give thioether 28.12. 3-Ethyl-5-hydroxy-4-oxo-2-p-tolyl-3,4-dihydro-pyrimidine-6-carboxylic acid [1-(4-fluoro-phenyl)-cyclopropyl]-amide 28.13 is reacted with a dialkyl bromomethyl phosphonate 28.15 (Lancaster) and potassium carbonate, to produce the phosphonate 28.16. 5-Hydroxy-4-oxo-3-propyl-2-p-tolyl-3,4-dihydro-pyrimidine-6-carboxylic acid (5-sulfamoyl-naphthalen-2-ylmethyl)-amide 28.17 is alkylated with 2-chloroethyl dialkylphosphonate reagent 28.19 to give phosphonate pyrimidinone 28.20.

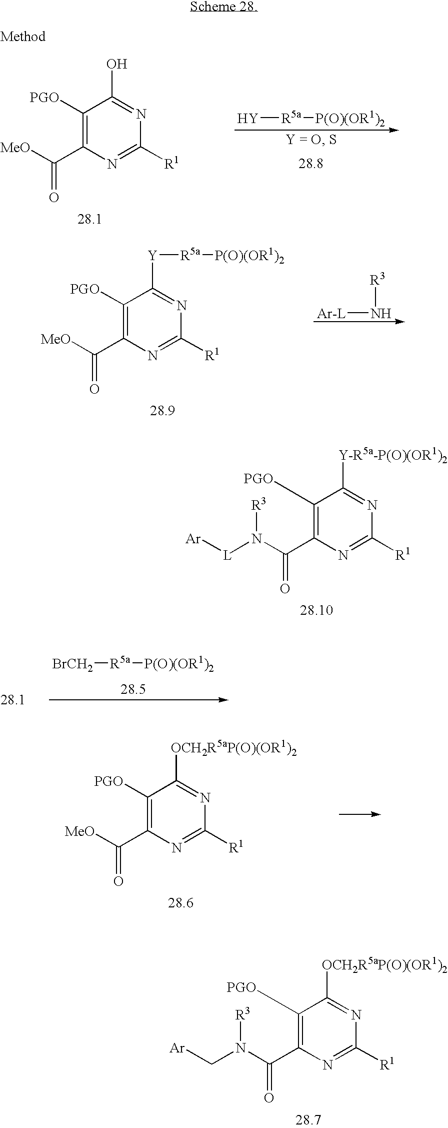

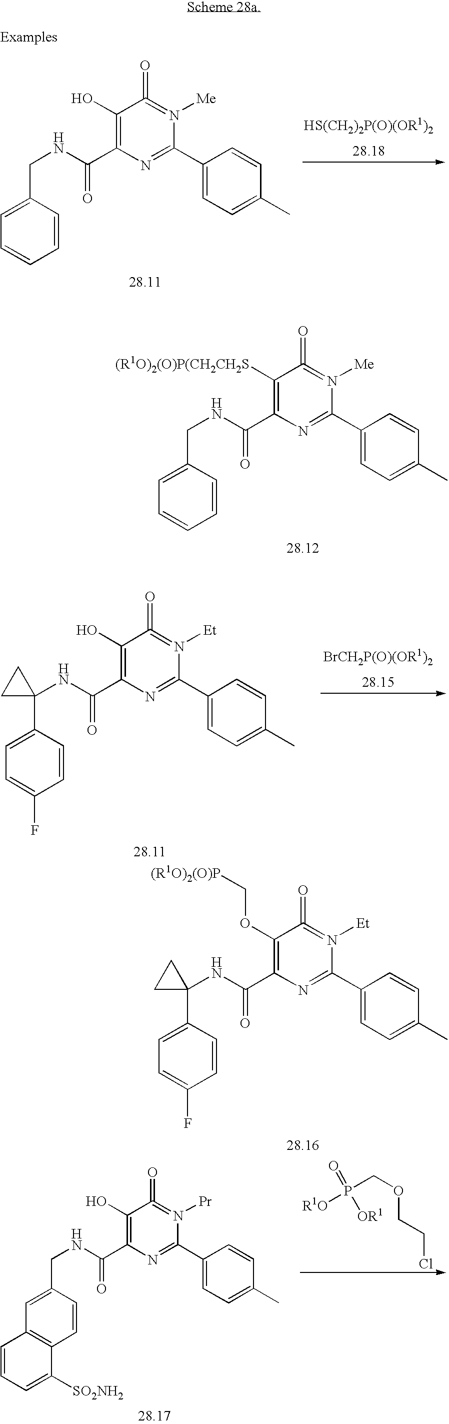

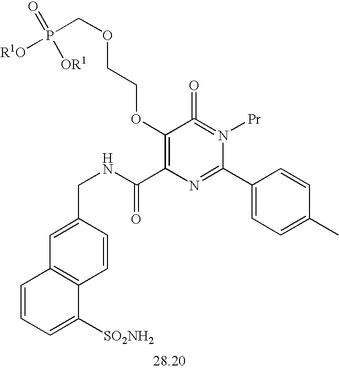

Scheme 29 illustrates the preparation of phosphonate esters Ia in which the phosphonate is attached either directly, or by means of a saturated or unsaturated carbon chain at the 2-position. In this procedure, a bromo-substituted anhydride 29.1 is converted, as described above, into the phenol-protected amide 29.2. The product is then subjected to a Heck coupling reaction, in the presence of a palladium (0) catalyst, as described in Scheme 4, with a dialkyl alkenyl phosphonate 29.3, to afford the phosphonate 29.4. Optionally, the olefinic bond is reduced, as described in Scheme 4, to yield the saturated analog 29.5.

Alternatively, the bromo-substituted amide 29.1 is coupled, as described in Scheme 3, with a dialkyl phosphite, in the presence of a palladium (0) catalyst, to generate, after deprotection of the phenolic hydroxyl group, the amide phosphonate 29.6.

For example, 2-bromo-4,5-dihydroxy-pyrimidine-6-carboxylic acid 4-trifluoromethyl-benzylamide 29.8. This compound is then reacted, in dimethylformamide solution at 80° C., with one molar equivalent of a dialkyl vinyl phosphonate 29.9, (Aldrich), triethylamine and a catalytic amount of tetrakis(triphenylphosphine)palladium(0) to yield, after desilylation, the unsaturated phosphonate 29.10. The product is then reacted with diimide, prepared by basic hydrolysis of diethyl azodicarboxylate, as described in Angew. Chem. Int. Ed., 4, 271, 1965, to yield the saturated product 29.11.

Alternatively, 29.8 is reacted in toluene solution at ca. 100° C., with one molar equivalent of a dialkyl phosphite 29.2, triethylamine and 3 mol % tetrakis(triphenylphosphine)palladium(0), to give, after desilylation, the phosphonate product 29.12.

Using the above procedures, but employing, in place of the anhydride 29.7, different anhydrides 29.1, and/or different phosphonates 29.3, the corresponding products 29.4, 29.5 and 29.6 are obtained.

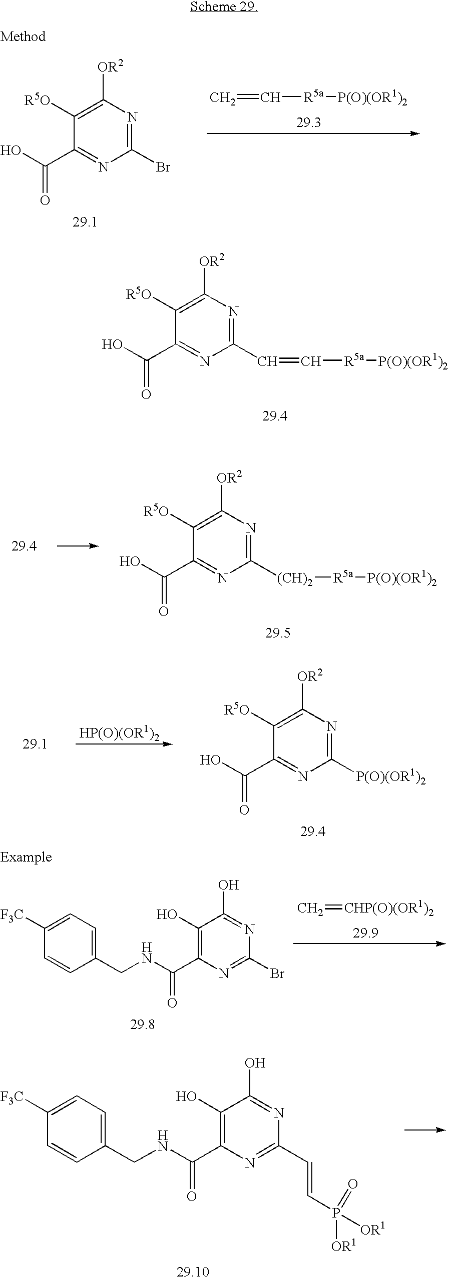

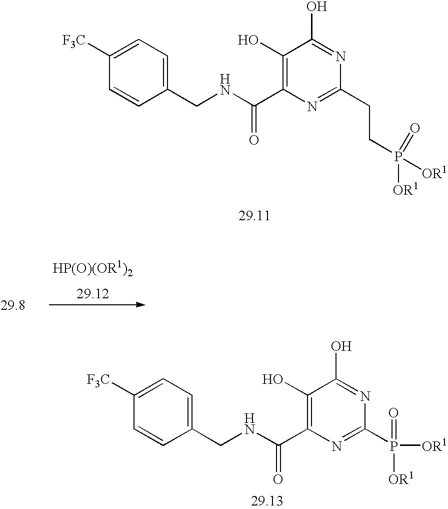

Scheme 30 illustrates the preparation of phosphonate esters IIa in which the phosphonate is attached by means of a saturated or unsaturated carbon link at the 2-position. In this procedure, the amide 30.2 is condensed, under basic conditions, with a diallyl formyl-substituted phosphonate 30.3, to afford the unsaturated phosphonate 30.4. The reaction is conducted at from ambient temperature to about 100° C., in a polar aprotic solvent such as dimethylformamide or dioxane, in the presence of a base such as sodium hydride, potassium tert butoxide or lithium hexamethyldisilazide. Optionally, the product 30.4 is reduced, as described in Scheme 4, to afford the saturated analog 30.5.

For example, 3-(4-methoxy-benzyl)-2-methyl-4-oxo-5-triisopropylsilanyloxy-3,4-dihydro-pyrimidine-6-carboxylic acid (3,5-dichloro-benzyl)-ethyl-amide 30.7 is reacted, in dimethylformamide solution at 60° C., with one molar equivalent of a diallyl formylmethyl phosphonate 30.8 (Aurora) and sodium hydride, to give, after desilylation, the unsaturated phosphonate 30.9. The product is then reacted with diimide, prepared by basic hydrolysis of diethyl azodicarboxylate, as described in Angew. Chem. Int. Ed., 4, 271, 1965, to yield the saturated phosphonate 30.10.

Using the above procedures, but employing, in place of the anhydride 30.6, different anhydrides 30.1, and/or different phosphonates 30.3, the corresponding products 30.4, and 30.5 are obtained.

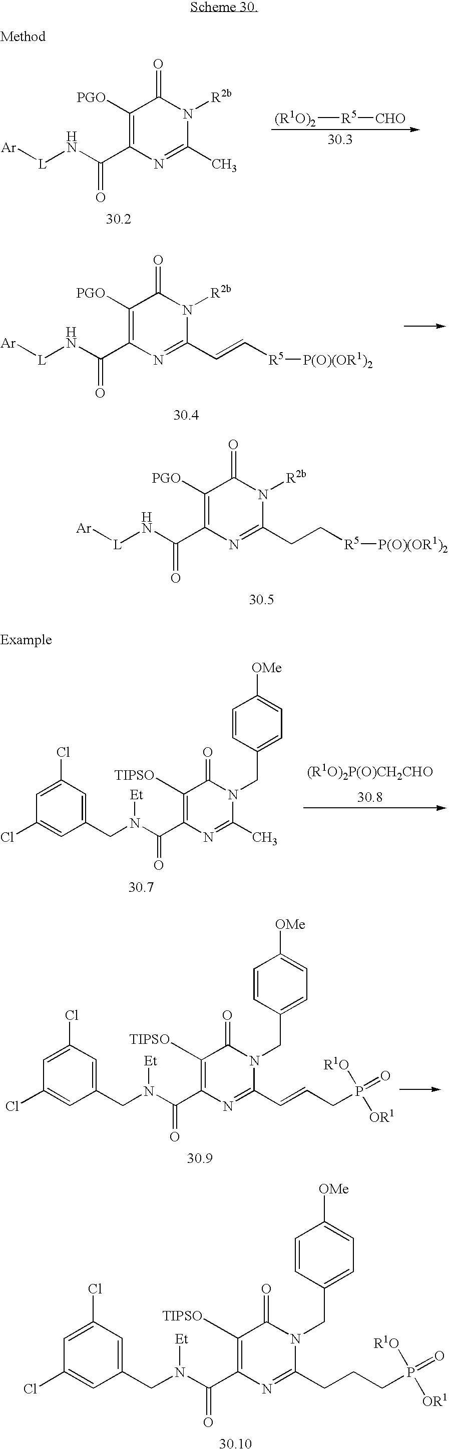

Scheme 31 illustrates the preparation of phosphonate esters Ia in which the phosphonate is attached by means of an oxime linkage at the 2-position. In this procedure, a 2-methyl, 6-amide 31.2 is brominated to give the 2-bromomethyl compound 31.3. Oxidation, as described in Scheme 26, of 31.3 affords the corresponding aldehyde 31.4. The aldehyde 31.4 is then converted, by reaction with hydroxylamine, into the oxime 31.5. The latter compound is then reacted, in a polar solvent such as tetrahydrofuran or dimethylformamide, in the presence of a base such as sodium hydroxide or potassium carbonate, with a dialkyl bromomethyl-substituted phosphonate 31.6, to prepare, after deprotection of the phenolic hydroxyl group, the oxime derivative 31.7.

For example, 2-formyl-4,5-dimethoxy-pyrimidine-6-carboxylic acid 4-fluoro-benzylamide 31.9 is reacted in tetrahydrofuran solution with three molar equivalents of hydroxylamine hydrochloride and sodium acetate, to produce 2-(hydroxyimino-methyl)-4,5-dimethoxy-pyrimidine-6-carboxylic acid 4-fluoro-benzylamide 31.10, which is then reacted in dioxane solution at ambient temperature, with one molar equivalent of a dialkyl bromopropyl phosphonate 31.11 (Synthelec) and potassium carbonate, to yield, after desilylation of the phenolic hydroxyl group, the oxime ether 31.12.

Also for example, a 2-phosphonate Formula Ia compound can be prepared with a morpholino linkage. The 5-hydroxyl of 3-[4-(4-Fluoro-benzylcarbamoyl)-5-hydroxy-3-methyl-4-oxo-3,4-dihydro-pyrimidin-2-yl]-morpholine-4-carboxylic acid tert-butyl ester 31.13 can be esterified as the 2-iodobenzoate to give 31.14. The Boc group can be removed under acidic conditions from 31.14 and the amino group of 2-iodo-benzoic acid 4-(4-fluoro-benzylcarbamoyl)-1-methyl-2-morpholin-3-yl-6-oxo-1,6-dihydro-pyrimidin-5-yl ester 31.15 may be condensed with aldehyde 31.16 to give 31.17 by reductive amination with sodium cyanoborohydride. The 2-iodobenzoate group may be removed under mild oxidative conditions, following the methods of R. Moss etal, Tetrahedron Letters, 28, 5005 (1989), to give morpholino phosphonate 31.18.

Using the above procedures, but employing, in place of the anhydride 31.8, different anhydrides 31.1, and/or different phosphonates 31.6, the corresponding products 31.7 are obtained.

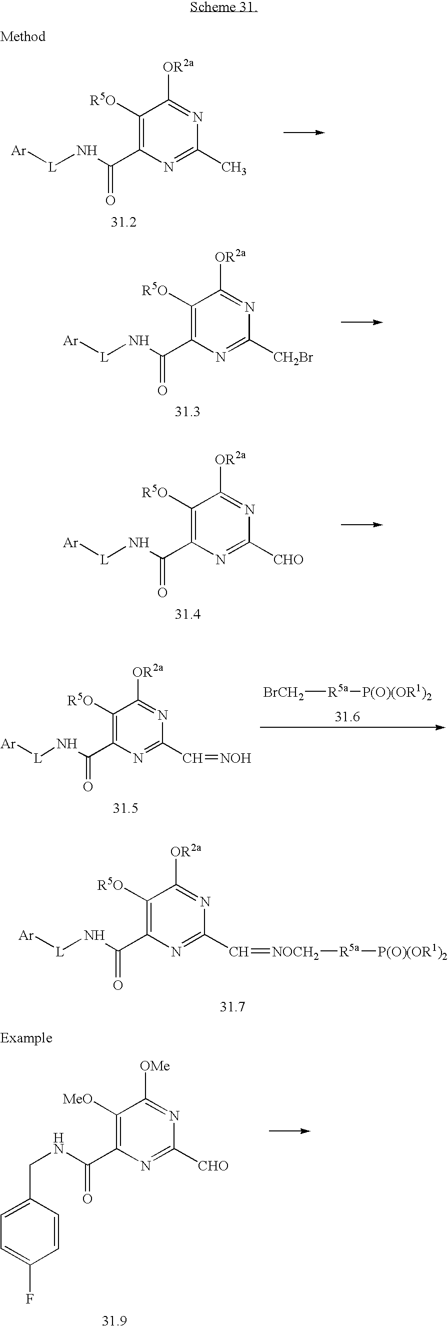

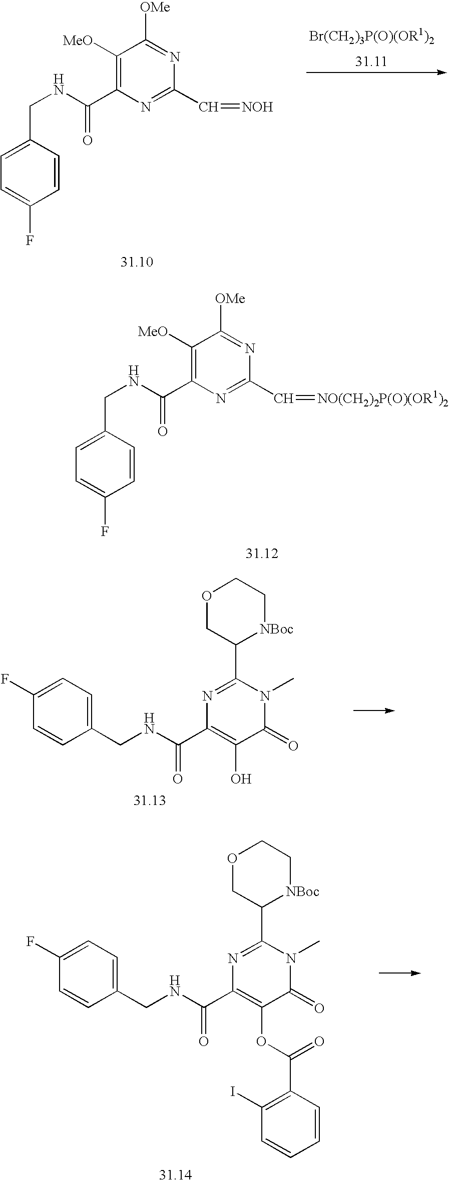

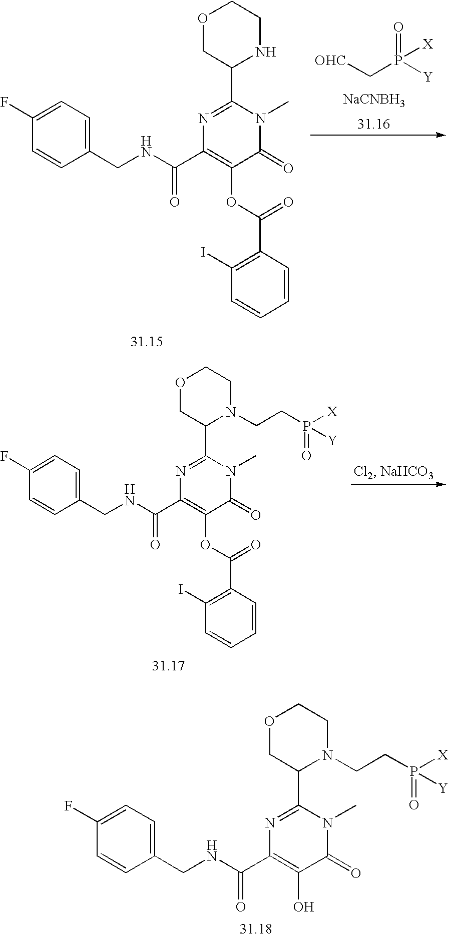

### Interconversions of the Phosphonates R-Link-P(O)(OR¹)₂, R-Link-P(O)(OR¹)(OH) and R-Link-P(O)(OH)₂.

Schemes 1-31 described the preparation of phosphonate esters of the general structure R-link-P(O)(OR¹)₂, in which the groups R¹ may be the same or different. The R¹ groups attached to a phosphonate ester Ia-d and IIa-d, or to precursors thereto, may be changed using established chemical transformations. The interconversion reactions of phosphonates are illustrated in Scheme 32. The group R in Scheme 32 represents the substructure to which the substituent link-P(O)(OR¹)₂ is attached, either in the compounds Ia-d and IIa-d, or in precursors thereto. The R¹ group may be changed, using the procedures described below, either in the precursor compounds, or in the esters Ia-d and IIa-d. The methods employed for a given phosphonate transformation depend on the nature of the substituent R¹, and of the substrate to which the phosphonate group is attached. The preparation and hydrolysis of phosphonate esters is described in Organic Phosphorus Compounds, G. M. Kosolapoff, L. Maeir, eds, Wiley, 1976, p. 9ff.

The conversion of a phosphonate diester 32.1 into the corresponding phosphonate monoester 32.2 (Scheme 32, Reaction 1) is accomplished by a number of methods. For example, the ester 32.1 in which R¹ is an aralkyl group such as benzyl, is converted into the monoester compound 32.2 by reaction with a tertiary organic base such as diazabicyclooctane (DABCO) or quinuclidine, as described in J. Org. Chem., 1995, 60, 2946. The reaction is performed in an inert hydrocarbon solvent such as toluene or xylene, at about 110° C. The conversion of the diester 32.1 in which R¹ is an aryl group such as phenyl, or an alkenyl group such as allyl, into the monoester 32.2 is effected by treatment of the ester 32.1 with a base such as aqueous sodium hydroxide in acetonitrile or lithium hydroxide in aqueous tetrahydrofuran. Phosphonate diesters 32.1 in which one of the groups R¹ is aralkyl, such as benzyl, and the other is alkyl, is converted into the monoesters 32.2 in which R¹ is alkyl by hydrogenation, for example using a palladium on carbon catalyst. Phosphonate diesters in which both of the groups R¹ are alkenyl, such as allyl, is converted into the monoester 32.2 in which R¹ is alkenyl, by treatment with chlorotris(triphenylphosphine)rhodium (Wilkinson's catalyst) in aqueous ethanol at reflux, optionally in the presence of diazabicyclooctane, for example by using the procedure described in J. Org. Chem., 38, 3224, 1973 for the cleavage of allyl carboxylates.

The conversion of a phosphonate diester 32.1 or a phosphonate monoester 32.2 into the corresponding phosphonic acid 32.3 (Scheme 32, Reactions 2 and 3) can effected by reaction of the diester or the monoester with trimethylsilyl bromide, as described in J. Chem. Soc., Chem. Comm., 739, 1979. The reaction is conducted in an inert solvent such as, for example, dichloromethane, optionally in the presence of a silylating agent such as bis(trimethylsilyl)trifluoroacetamide, at ambient temperature. A phosphonate monoester 32.2 in which R¹ is aralkyl such as benzyl, is converted into the corresponding phosphonic acid 32.3 by hydrogenation over a palladium catalyst, or by treatment with hydrogen chloride in an ethereal solvent such as dioxane. A phosphonate monoester 32.2 in which R¹ is alkenyl such as, for example, allyl, is converted into the phosphonic acid 32.3 by reaction with Wilkinson's catalyst in an aqueous organic solvent, for example in 15% aqueous acetonitrile, or in aqueous ethanol, for example using the procedure described in Helv. Chim. Acta., 68, 618, 1985. Palladium catalyzed hydrogenolysis of phosphonate esters 32.1 in which R¹ is benzyl is described in J. Org. Chem., 24, 434, 1959. Platinum-catalyzed hydrogenolysis of phosphonate esters 32.1 in which R¹ is phenyl is described in J. Am. Chem. Soc., 78, 2336, 1956.

The conversion of a phosphonate monoester 32.2 into a phosphonate diester 32.1 (Scheme 32, Reaction 4) in which the newly introduced R¹ group is alkyl, aralkyl, haloalkyl such as chloroethyl, or aralkyl is effected by a number of reactions in which the substrate 32.2 is reacted with a hydroxy compound R¹OH, in the presence of a coupling agent. Suitable coupling agents are those employed for the preparation of carboxylate esters, and include a carbodiimide such as dicyclohexylcarbodiimide, in which case the reaction is preferably conducted in a basic organic solvent such as pyridine, or (benzotriazol-1-yloxy)tripyrrolidinophosphonium hexafluorophosphate (PYBOP, Sigma), in which case the reaction is performed in a polar solvent such as dimethylformamide, in the presence of a tertiary organic base such as diisopropylethylamine, or Aldrithiol-2 (Aldrich) in which case the reaction is conducted in a basic solvent such as pyridine, in the presence of a triaryl phosphine such as triphenylphosphine. Alternatively, the conversion of the phosphonate monoester 32.2 to the diester 32.1 is effected by the use of the Mitsunobu reaction, as described above (Scheme 7). The substrate is reacted with the hydroxy compound R¹OH, in the presence of diethyl azodicarboxylate and a triarylphosphine such as triphenyl phosphine. Alternatively, the phosphonate monoester 32.2 is transformed into the phosphonate diester 32.1, in which the introduced R¹ group is alkenyl or aralkyl, by reaction of the monoester with the halide R¹Br, in which R¹ is as alkenyl or aralkyl. The alkylation reaction is conducted in a polar organic solvent such as dimethylformamide or acetonitrile, in the presence of a base such as cesium carbonate. Alternatively, the phosphonate monoester is transformed into the phosphonate diester in a two step procedure. In the first step, the phosphonate monoester 32.2 is transformed into the chloro analog RP(O)(OR¹)Cl by reaction with thionyl chloride or oxalyl chloride and the like, as described in Organic Phosphorus Compounds, G. M. Kosolapoff, L. Maeir, eds, Wiley, 1976, p. 17, and the thus-obtained product RP(O)(OR¹)Cl is then reacted with the hydroxy compound R¹OH, in the presence of a base such as triethylamine, to afford the phosphonate diester 32.1.

A phosphonic acid R-link-P(O)(OH)₂ is transformed into a phosphonate monoester RP(O)(OR¹)(OH) (Scheme 32, Reaction 5) by means of the methods described above of for the preparation of the phosphonate diester R-link-P(O)(OR¹)₂ 32.1, except that only one molar proportion of the component R¹OH or R¹Br is employed.

A phosphonic acid R-link-P(O)(OH)₂ 32.3 is transformed into a phosphonate diester R-link-P(O)(OR¹)₂ 32.1 (Scheme 32, Reaction 6) by a coupling reaction with the hydroxy compound R¹OH, in the presence of a coupling agent such as Aldrithiol-2 (Aldrich) and triphenylphosphine. The reaction is conducted in a basic solvent such as pyridine. Alternatively, phosphonic acids 32.3 is transformed into phosphonic esters 32.1 in which R¹ is aryl, by means of a coupling reaction employing, for example, dicyclohexylcarbodiimide in pyridine at ca 70° C. Alternatively, phosphonic acids 32.3 is transformed into phosphonic esters 32.1 in which R¹ is alkenyl, by means of an alkylation reaction. The phosphonic acid is reacted with the alkenyl bromide R¹Br in a polar organic solvent such as acetonitrile solution at reflux temperature, the presence of a base such as cesium carbonate, to afford the phosphonic ester 32.1.

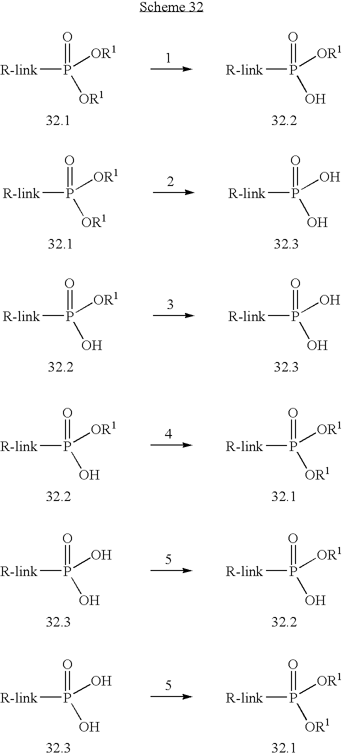

### Preparation of Carbamates.

The phosphonate esters 1-9 may contain a carbamate linkage. The preparation of carbamates is described in Comprehensive Organic Functional Group Transformations, A. R. Katritzky, ed., Pergamon, 1995, Vol. 6, p. 416ff, and in Organic Functional Group Preparations, by S. R. Sandler and W. Karo, Academic Press, 1986, p. 260ff.

Scheme 33 illustrates various methods by which the carbamate linkage is synthesized. As shown in Scheme 33, in the general reaction generating carbamates, a carbinol 33.1, is converted into the activated derivative 33.2 in which Lv is a leaving group such as halo, imidazolyl, benztriazolyl and the like, as described herein. The activated derivative 33.2 is then reacted with an amine 33.3, to afford the carbamate product 33.4. Examples 1-7 in Scheme 33 depict methods by which the general reaction is effected. Examples 8-10 illustrate alternative methods for the preparation of carbamates.

Scheme 33, Example 1 illustrates the preparation of carbamates employing a chloroformyl derivative of the carbinol 33.5. In this procedure, the carbinol 33.5 is reacted with phosgene, in an inert solvent such as toluene, at about 0°, as described in Org. Syn. Coll. Vol. 3, 167, 1965, or with an equivalent reagent such as trichloromethoxy chloroformate, as described in Org. Syn. Coll. Vol. 6, 715, 1988, to afford the chloroformate 33.6. The latter compound is then reacted with the amine component 33.3, in the presence of an organic or inorganic base, to afford the carbamate 33.7. For example, the chloroformyl compound 33.6 is reacted with the amine 33.3 in a water-miscible solvent such as tetrahydrofuran, in the presence of aqueous sodium hydroxide, as described in Org. Syn. Coll. Vol. 3, 167, 1965, to yield the carbamate 33.7. Alternatively, the reaction is performed in dichloromethane in the presence of an organic base such as diisopropylethylamine or dimethylaminopyridine.

Scheme 33, Example 2 depicts the reaction of the chloroformate compound 33.6 with imidazole to produce the imidazolide 33.8. The imidazolide product is then reacted with the amine 33.3 to yield the carbamate 33.7. The preparation of the imidazolide is performed in an aprotic solvent such as dichloromethane at 0°, and the preparation of the carbamate is conducted in a similar solvent at ambient temperature, optionally in the presence of a base such as dimethylaminopyridine, as described in J. Med. Chem., 1989, 32, 357.

Scheme 33 Example 3, depicts the reaction of the chloroformate 33.6 with an activated hydroxyl compound R″OH, to yield the mixed carbonate ester 33.10. The reaction is conducted in an inert organic solvent such as ether or dichloromethane, in the presence of a base such as dicyclohexylamine or triethylamine. The hydroxyl component R″OH is selected from the group of compounds 33.19-33.24 shown in Scheme 33, and similar compounds. For example, if the component R″OH is hydroxybenztriazole 33.19, N-hydroxysuccinimide 33.20, or pentachlorophenol, 33.21, the mixed carbonate 33.10 is obtained by the reaction of the chloroformate with the hydroxyl compound in an ethereal solvent in the presence of dicyclohexylamine, as described in Can. J. Chem., 1982, 60, 976. A similar reaction in which the component R″OH is pentafluorophenol 33.22 or 2-hydroxypyridine 33.23 is performed in an ethereal solvent in the presence of triethylamine, as described in Syn., 1986, 303, and Chem. Ber. 118, 468, 1985.

Scheme 33 Example 4 illustrates the preparation of carbamates in which an alkyloxycarbonylimidazole 33.8 is employed. In this procedure, a carbinol 33.5 is reacted with an equimolar amount of carbonyl diimidazole 33.11 to prepare the intermediate 33.8. The reaction is conducted in an aprotic organic solvent such as dichloromethane or tetrahydrofuran. The acyloxyimidazole 33.8 is then reacted with an equimolar amount of the amine R′NH₂ to afford the carbamate 33.7. The reaction is performed in an aprotic organic solvent such as dichloromethane, as described in Tet. Lett., 42, 2001, 5227, to afford the carbamate 33.7.

Scheme 33, Example 5 illustrates the preparation of carbamates by means of an intermediate alkoxycarbonylbenztriazole 33.13. In this procedure, a carbinol ROH is reacted at ambient temperature with an equimolar amount of benztriazole carbonyl chloride 33.12, to afford the alkoxycarbonyl product 33.13. The reaction is performed in an organic solvent such as benzene or toluene, in the presence of a tertiary organic amine such as triethylamine, as described in Synthesis., 1977, 704. The product is then reacted with the amine R′NH₂ to afford the carbamate 33.7. The reaction is conducted in toluene or ethanol, at from ambient temperature to about 80° C. as described in Synthesis, 1977, 704.

Scheme 33, Example 6 illustrates the preparation of carbamates in which a carbonate (R″O)₂CO, 33.14, is reacted with a carbinol 33.5 to afford the intermediate allyloxycarbonyl intermediate 33.15. The latter reagent is then reacted with the amine R′NH₂ to afford the carbamate 33.7. The procedure in which the reagent 33.15 is derived from hydroxybenztriazole 33.19 is described in Synthesis, 1993, 908; the procedure in which the reagent 33.15 is derived from N-hydroxysuccinimide 33.20 is described in Tet. Lett., 1992, 2781; the procedure in which the reagent 33.15 is derived from 2-hydroxypyridine 33.23 is described in Tet. Lett., 1991, 4251; the procedure in which the reagent 33.15 is derived from 4-nitrophenol 33.24 is described in Synthesis. 1993, 103. The reaction between equimolar amounts of the carbinol ROH and the carbonate 33.14 is conducted in an inert organic solvent at ambient temperature.

Scheme 33, Example 7 illustrates the preparation of carbamates from alkoxycarbonyl azides 33.16. In this procedure, an alkyl chloroformate 33.6 is reacted with an azide, for example sodium azide, to afford the alkoxycarbonyl azide 33.16. The latter compound is then reacted with an equimolar amount of the amine R′NH₂ to afford the carbamate 33.7. The reaction is conducted at ambient temperature in a polar aprotic solvent such as dimethylsulfoxide, for example as described in Synthesis, 1982, 404.

Scheme 33, Example 8 illustrates the preparation of carbamates by means of the reaction between a carbinol ROH and the chloroformyl derivative of an amine 33.17. In this procedure, which is described in Synthetic Organic Chemistry, R. B. Wagner, H. D. Zook, Wiley, 1953, p. 647, the reactants are combined at ambient temperature in an aprotic solvent such as acetonitrile, in the presence of a base such as triethylamine, to afford the carbamate 33.7.

Scheme 33, Example 9 illustrates the preparation of carbamates by means of the reaction between a carbinol ROH and an isocyanate 33.18. In this procedure, which is described in Synthetic Organic Chemistry, R. B. Wagner, H. D. Zook, Wiley, 1953, p. 645, the reactants are combined at ambient temperature in an aprotic solvent such as ether or dichloromethane and the like, to afford the carbamate 33.7.

Scheme 33, Example 10 illustrates the preparation of carbamates by means of the reaction between a carbinol ROH and an amine R′NH₂. In this procedure, which is described in Chem. Lett. 1972, 373, the reactants are combined at ambient temperature in an aprotic organic solvent such as tetrahydrofuran, in the presence of a tertiary base such as triethylamine, and selenium. Carbon monoxide is passed through the solution and the reaction proceeds to afford the carbamate 33.7.

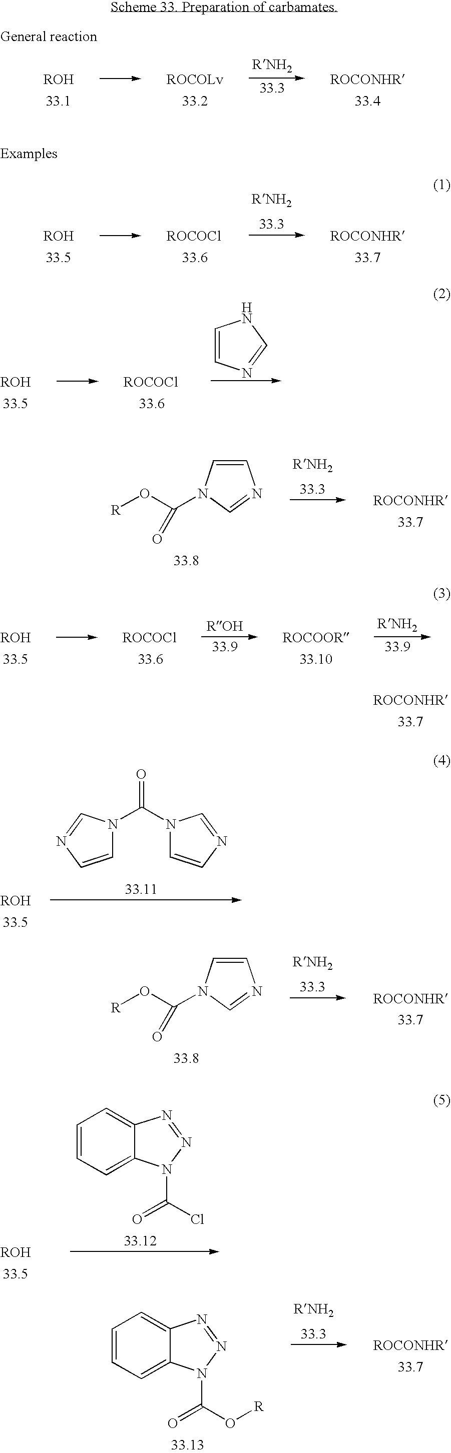

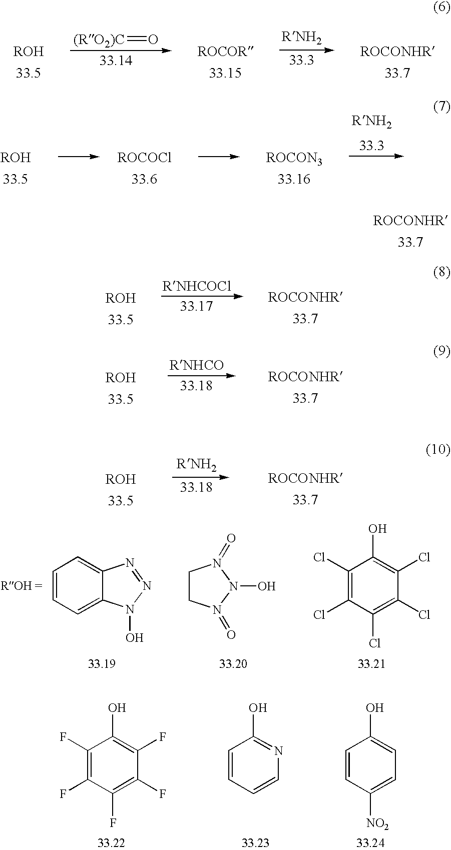

### Preparation of Carboalkoxy-Substituted Phosphonate Diamidates Monoamidates Diesters and Monoesters.

A number of methods are available for the conversion of phosphonic acids into amidates and esters. In one group of methods, the phosphonic acid is either converted into an isolated activated intermediate such as a phosphoryl chloride, or the phosphonic acid is activated in situ for reaction with an amine or a hydroxy compound.

The conversion of phosphonic acids into phosphoryl chlorides is accomplished by reaction with thionyl chloride, for example as described in J. Gen. Chem. USSR, 1983, 53, 480, Zh. Obschei Khim., 1958, 28, 1063, or J. Org Chem., 1994, 59, 6144, or by reaction with oxalyl chloride, as described in J. Am. Chem. Soc., 1994, 116, 3251, or J. Org. Chem., 1994, 59, 6144, or by reaction with phosphorus pentachloride, as described in J. Org. Chem., 2001, 66, 329, or in J. Med. Chem., 1995, 38, 1372. The resultant phosphoryl chlorides are then reacted with amines or hydroxy compounds in the presence of a base to afford the amidate or ester products.

Phosphonic acids are converted into activated imidazolyl derivatives by reaction with carbonyl diimidazole, as described in J. Chem. Soc., Chem. Comm., 1991, 312, or Nucleosides & Nucleotides 2000, 19, 1885. Activated sulfonyloxy derivatives are obtained by the reaction of phosphonic acids with trichloromethylsulfonyl chloride, as described in J. Med. Chem. 1995, 38, 4958, or with triisopropylbenzenesulfonyl chloride, as described in Tet. Lett., 1996, 7857, or Bioorg. Med. Chem. Lett., 1998, 8, 663. The activated sulfonyloxy derivatives are then reacted with amines or hydroxy compounds to afford amidates or esters.

Alternatively, the phosphonic acid and the amine or hydroxy reactant are combined in the presence of a diimide coupling agent. The preparation of phosphonic amidates and esters by means of coupling reactions in the presence of dicyclohexyl carbodiimide is described, for example, in J. Chem. Soc., Chem. Comm., 1991, 312, or J. Med. Chem., 1980, 23, 1299 or Coll. Czech. Chem. Comm., 1987, 52, 2792. The use of ethyl dimethylaminopropyl carbodiimide for activation and coupling of phosphonic acids is described in Tet. Lett., 2001, 42, 8841, or Nucleosides & Nucleotides, 2000, 19, 1885.

A number of additional coupling reagents have been described for the preparation of amidates and esters from phosphonic acids. The agents include Aldrithiol-2, and PYBOP and BOP, as described in J. Org. Chem., 1995, 60, 5214, and J. Med. Chem., 1997, 40, 3842, mesitylene-2-sulfonyl-3-nitro-1,2,4-triazole (MSNT), as described in J. Med. Chem., 1996, 39, 4958, diphenylphosphoryl azide, as described in J. Org. Chem., 1984, 49, 1158, 1-(2,4,6-triisopropylbenzenesulfonyl-3-nitro-1,2,4-triazole (TPSNT) as described in Bioorg. Med. Chem. Lett., 1998, 8, 1013, bromotris(dimethylamino)phosphonium hexafluorophosphate (BroP), as described in Tet. Lett., 1996, 37, 3997, 2-chloro-5,5-dimethyl-2-oxo-1,3,2-dioxaphosphinane, as described in Nucleosides Nucleotides 1995, 14, 871, and diphenyl chlorophosphate, as described in J. Med. Chem., 1988, 31, 1305.

Phosphonic acids are converted into amidates and esters by means of the Mitsunobu reaction, in which the phosphonic acid and the amine or hydroxy reactant are combined in the presence of a triaryl phosphine and a diallyl azodicarboxylate. The procedure is described in Org. Lett., 2001, 3, 643, or J. Med. Chem., 1997, 40, 3842.

Phosphonic esters are also obtained by the reaction between phosphonic acids and halo compounds, in the presence of a suitable base. The method is described, for example, in Anal. Chem., 1987, 59, 1056, or J. Chem. Soc. Perkin Trans., I, 1993, 19, 2303, or J. Med. Chem., 1995, 38, 1372, or Tet. Lett., 2002, 43, 1161.

Schemes 34-37 illustrate the conversion of phosphonate esters and phosphonic acids into carboalkoxy-substituted phosphondiamidates (Scheme 34), phosphonamidates (Scheme 35), phosphonate monoesters (Scheme 36) and phosphonate diesters, (Scheme 37). Scheme 38 illustrates synthesis of gem-dialkyl amino phosphonate reagents.

Scheme 34 illustrates various methods for the conversion of phosphonate diesters 34.1 into phosphondiamidates 34.5. The diester 34.1, prepared as described previously, is hydrolyzed, either to the monoester 34.2 or to the phosphonic acid 34.6. The methods employed for these transformations are described above. The monoester 34.2 is converted into the monoamidate 34.3 by reaction with an aminoester 34.9, in which the group R² is H or alkyl, the group R^{4b} is an alkylene moiety such as, for example, CHCH₃, CHPr^{I}, CH(CH₂Ph), CH₂CH(CH₃) and the like, or a group present in natural or modified aminoacids, and the group R^{5b} is alkyl. The reactants are combined in the presence of a coupling agent such as a carbodiimide, for example dicyclohexyl carbodiimide, as described in J. Am. Chem. Soc., 1957, 79, 3575, optionally in the presence of an activating agent such as hydroxybenztriazole, to yield the amidate product 34.3. The amidate-forming reaction is also effected in the presence of coupling agents such as BOP, as described in J. Org. Chem., 1995, 60, 5214, Aldrithiol, PYBOP and similar coupling agents used for the preparation of amides and esters. Alternatively, the reactants 34.2 and 34.9 are transformed into the monoamidate 34.3 by means of a Mitsunobu reaction. The preparation of amidates by means of the Mitsunobu reaction is described in J. Med. Chem., 1995, 38, 2742. Equimolar amounts of the reactants are combined in an inert solvent such as tetrahydrofuran in the presence of a triaryl phosphine and a dialkyl azodicarboxylate. The thus-obtained monoamidate ester 34.3 is then transformed into amidate phosphonic acid 34.4. The conditions used for the hydrolysis reaction depend on the nature of the R¹ group, as described previously. The phosphonic acid amidate 34.4 is then reacted with an aminoester 34.9, as described above, to yield the bisamidate product 34.5, in which the amino substituents are the same or different.

An example of this procedure is shown in Scheme 34, Example 1. In this procedure, a dibenzyl phosphonate 34.14 is reacted with diazabicyclooctane (DABCO) in toluene at reflux, as described in J. Org. Chem., 1995, 60, 2946, to afford the monobenzyl phosphonate 34.15. The product is then reacted with equimolar amounts of ethyl alaninate 34.16 and dicyclohexyl carbodiimide in pyridine, to yield the amidate product 34.17. The benzyl group is then removed, for example by hydrogenolysis over a palladium catalyst, to give the monoacid product 34.18. This compound is then reacted in a Mitsunobu reaction with ethyl leucinate 34.19, triphenyl phosphine and diethylazodicarboxylate, as described in J. Med. Chem., 1995, 38, 2742, to produce the bisamidate product 34.20.

Using the above procedures, but employing in place of ethyl leucinate 34.19 or ethyl alaninate 34.16, different aminoesters 34.9, the corresponding products 34.5 are obtained.

Alternatively, the phosphonic acid 34.6 is converted into the bisamidate 34.5 by use of the coupling reactions described above. The reaction is performed in one step, in which case the nitrogen-related substituents present in the product 34.5 are the same, or in two steps, in which case the nitrogen-related substituents can be different.

An example of the method is shown in Scheme 34, Example 2. In this procedure, a phosphonic acid 34.6 is reacted in pyridine solution with excess ethyl phenylalaninate 34.21 and dicyclohexylcarbodiimide, for example as described in J. Chem. Soc., Chem. Comm., 1991, 1063, to give the bisamidate product 34.22.

Using the above procedures, but employing, in place of ethyl phenylalaninate, different aminoesters 34.9, the corresponding products 34.5 are obtained.

As a further alternative, the phosphonic acid 34.6 is converted into the mono or bis-activated derivative 34.7, in which Lv is a leaving group such as chloro, imidazolyl, triisopropylbenzenesulfonyloxy etc. The conversion of phosphonic acids into chlorides 34.7 (Lv=Cl) is effected by reaction with thionyl chloride or oxalyl chloride and the like, as described in Organic Phosphorus Compounds. G. M. Kosolapoff, L. Maeir, eds, Wiley, 1976, p. 17. The conversion of phosphonic acids into monoimidazolides 34.7 (Lv=imidazolyl) is described in J. Med. Chem., 2002, 45, 1284 and in J. Chem. Soc. Chem. Comm., 1991, 312. Alternatively, the phosphonic acid is activated by reaction with triisopropylbenzenesulfonyl chloride, as described in Nucleosides and Nucleotides, 2000, 10, 1885. The activated product is then reacted with the aminoester 34.9, in the presence of a base, to give the bisamidate 34.5. The reaction is performed in one step, in which case the nitrogen substituents present in the product 34.5 are the same, or in two steps, via the intermediate 34.11, in which case the nitrogen substituents can be different.

Examples of these methods are shown in Scheme 34, Examples 3 and 5. In the procedure illustrated in Scheme 34, Example 3, a phosphonic acid 34.6 is reacted with ten molar equivalents of thionyl chloride, as described in Zh. Obschei Khim., 1958, 28, 1063, to give the dichloro compound 34.23. The product is then reacted at reflux temperature in a polar aprotic solvent such as acetonitrile, and in the presence of a base such as triethylamine, with butyl serinate 34.24 to afford the bisamidate product 34.25.

Using the above procedures, but employing, in place of butyl serinate 34.24, different aminoesters 34.9, the corresponding products 34.5 are obtained.

In the procedure illustrated in Scheme 34, Example 5, the phosphonic acid 34.6 is reacted, as described in J. Chem. Soc. Chem. Comm., 1991, 312, with carbonyl diimidazole to give the imidazolide 34.32. The product is then reacted in acetonitrile solution at ambient temperature, with one molar equivalent of ethyl alaninate 34.33 to yield the monodisplacement product 34.34. The latter compound is then reacted with carbonyl diimidazole to produce the activated intermediate 34.35, and the product is then reacted, under the same conditions, with ethyl N-methylalaninate 34.33a to give the bisamidate product 34.36.

Using the above procedures, but employing, in place of ethyl alaninate 34.33 or ethyl N-methylalaninate 34.33a, different aminoesters 34.9, the corresponding products 34.5 are obtained.

The intermediate monoamidate 34.3 is also prepared from the monoester 34.2 by first converting the monoester into the activated derivative 34.8 in which Lv is a leaving group such as halo, imidazolyl etc, using the procedures described above. The product 34.8 is then reacted with an aminoester 34.9 in the presence of a base such as pyridine, to give an intermediate monoamidate product 34.3. The latter compound is then converted, by removal of the R¹ group and coupling of the product with the aminoester 34.9, as described above, into the bisamidate 34.5.

An example of this procedure, in which the phosphonic acid is activated by conversion to the chloro derivative 34.26, is shown in Scheme 34, Example 4. In this procedure, the phosphonic monobenzyl ester 34.15 is reacted, in dichloromethane, with thionyl chloride, as described in Tet. Letters., 1994, 35, 4097, to afford the phosphoryl chloride 34.26. The product is then reacted in acetonitrile solution at ambient temperature with one molar equivalent of ethyl 3-amino-2-methylpropionate 34.27 to yield the monoamidate product 34.28. The latter compound is hydrogenated in ethylacetate over a 5% palladium on carbon catalyst to produce the monoacid product 34.29. The product is subjected to a Mitsunobu coupling procedure, with equimolar amounts of butyl alaninate 34.30, triphenyl phosphine, diethylazodicarboxylate and triethylamine in tetrahydrofuran, to give the bisamidate product 34.31.

Using the above procedures, but employing, in place of ethyl 3-amino-2-methylpropionate 34.27 or butyl alaninate 34.30, different amino esters 34.9, the corresponding products 34.5 are obtained.

The activated phosphonic acid derivative 34.7 is also converted into the bisamidate 34.5 via the diamino compound 34.10. The conversion of activated phosphonic acid derivatives such as phosphoryl chlorides into the corresponding amino analogs 34.10, by reaction with ammonia, is described in Organic Phosphorus Compounds, G. M. Kosolapoff, L. Maeir, eds, Wiley, 1976. The diamino compound 34.10 is then reacted at elevated temperature with a haloester 34.12 (Hal=halogen, i.e. F, Cl, Br, I), in a polar organic solvent such as dimethylformamide, in the presence of a base such as 4,4-dimethylaminopyridine (DMAP) or potassium carbonate, to yield the bisamidate 34.5.

An example of this procedure is shown in Scheme 34, Example 6. In this method, a dichlorophosphonate 34.23 is reacted with ammonia to afford the diamide 34.37. The reaction is performed in aqueous, aqueous alcoholic or alcoholic solution, at reflux temperature. The resulting diamino compound is then reacted with two molar equivalents of ethyl 2-bromo-3-methylbutyrate 34.38, in a polar organic solvent such as N-methylpyrrolidinone at ca 150° C., in the presence of a base such as potassium carbonate, and optionally in the presence of a catalytic amount of potassium iodide, to afford the bisamidate product 34.39.

Using the above procedures, but employing, in place of ethyl 2-bromo-3-methylbutyrate 34.38, different haloesters 34.12 the corresponding products 34.5 are obtained.

The procedures shown in Scheme 34 are also applicable to the preparation of bisamidates in which the aminoester moiety incorporates different functional groups. Scheme 34, Example 7 illustrates the preparation of bisamidates derived from tyrosine. In this procedure, the monoimidazolide 34.32 is reacted with propyl tyrosinate 34.40, as described in Example 5, to yield the monoamidate 34.41. The product is reacted with carbonyl diimidazole to give the imidazolide 34.42, and this material is reacted with a further molar equivalent of propyl tyrosinate to produce the bisamidate product 34.43.

Using the above procedures, but employing, in place of propyl tyrosinate 34.40, different aminoesters 34.9, the corresponding products 34.5 are obtained. The aminoesters employed in the two stages of the above procedure can be the same or different, so that bisamidates with the same or different amino substituents are prepared.

Scheme 35 illustrates methods for the preparation of phosphonate monoamidates.

In one procedure, a phosphonate monoester 34.1 is converted, as described in Scheme 34, into the activated derivative 34.8. This compound is then reacted, as described above, with an aminoester 34.9, in the presence of a base, to afford the monoamidate product 35.1.

The procedure is illustrated in Scheme 35, Example 1. In this method, a monophenyl phosphonate 35.7 is reacted with, for example, thionyl chloride, as described in J. Gen. Chem. USSR., 1983, 32, 367, to give the chloro product 35.8. The product is then reacted, as described in Scheme 34, with ethyl alaninate 35.9, to yield the amidate 35.10.

Using the above procedures, but employing, in place of ethyl alaninate 35.9, different aminoesters 34.9, the corresponding products 35.1 are obtained.

Alternatively, the phosphonate monoester 34.1 is coupled, as described in Scheme 34, with an aminoester 34.9 to produce the amidate 35.1. If necessary, the R¹ substituent is then altered, by initial cleavage to afford the phosphonic acid 35.2. The procedures for this transformation depend on the nature of the R¹ group, and are described above. The phosphonic acid is then transformed into the ester amidate product 35.3, by reaction with the hydroxy compound R³OH, in which the group R³ is aryl, heterocycle, alkyl, cycloalkyl, haloalkyl etc, using the same coupling procedures (carbodiimide, Aldrithiol-2, PYBOP, Mitsunobu reaction etc) described in Scheme 34 for the coupling of amines and phosphonic acids.

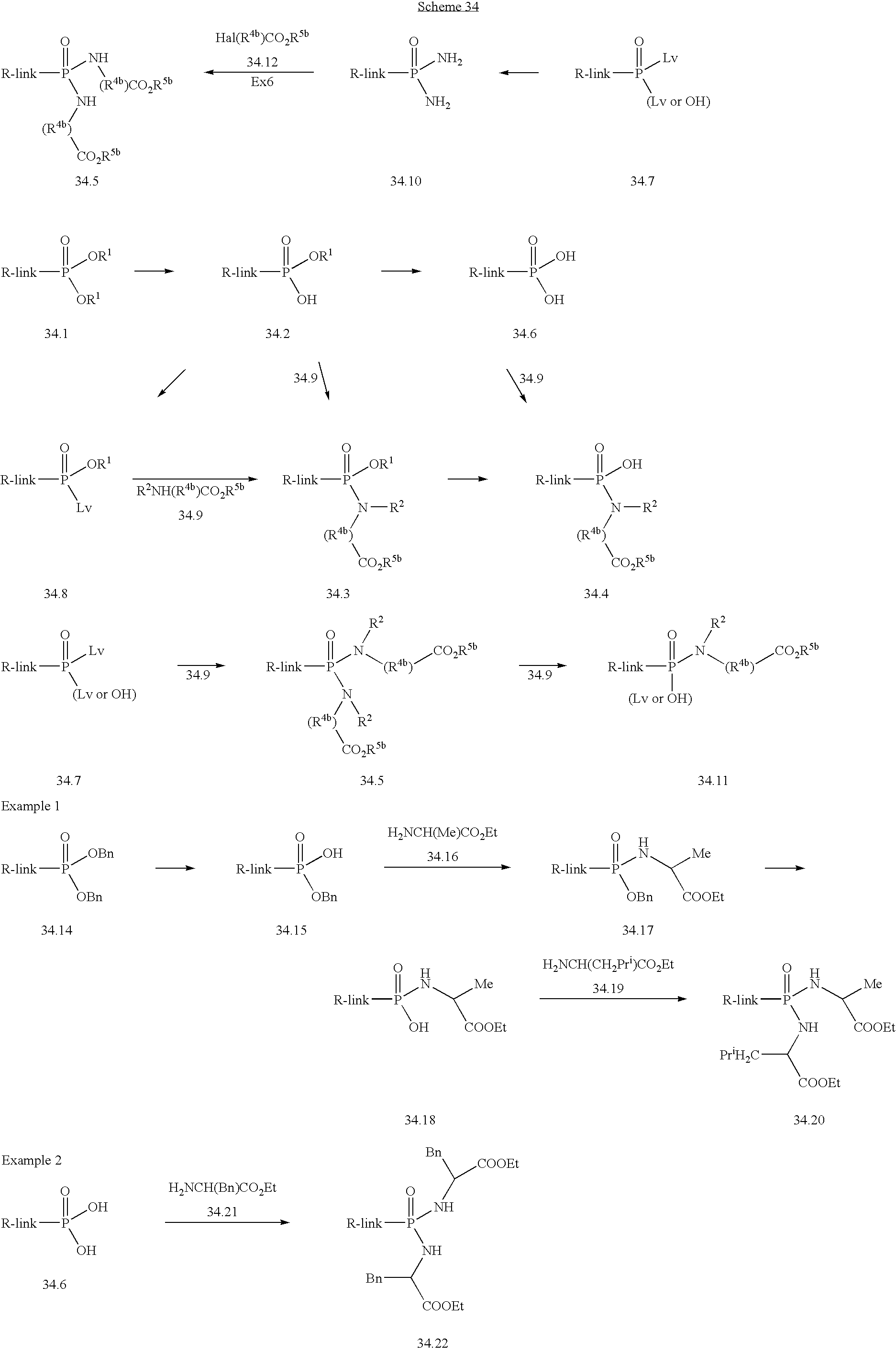

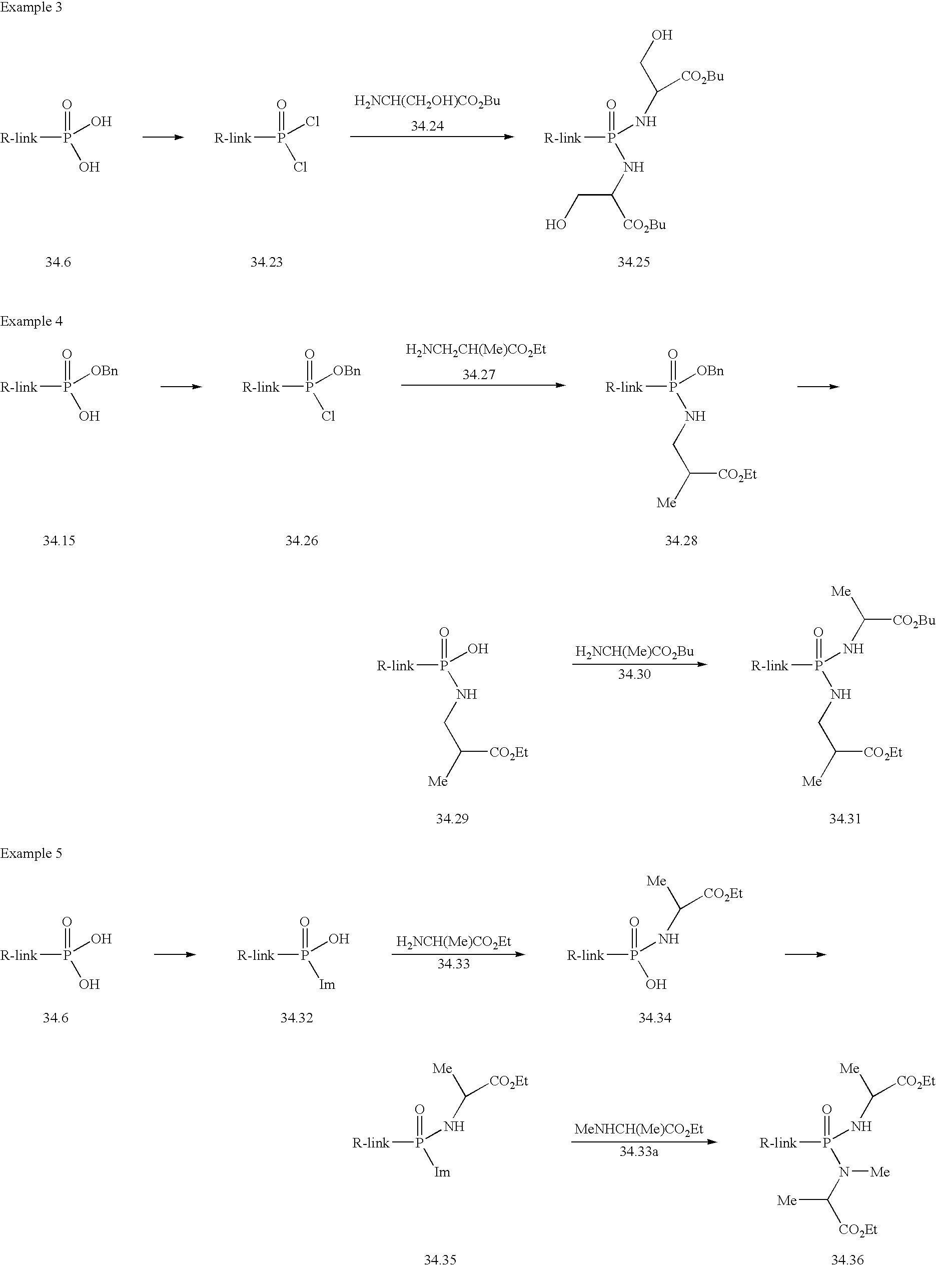

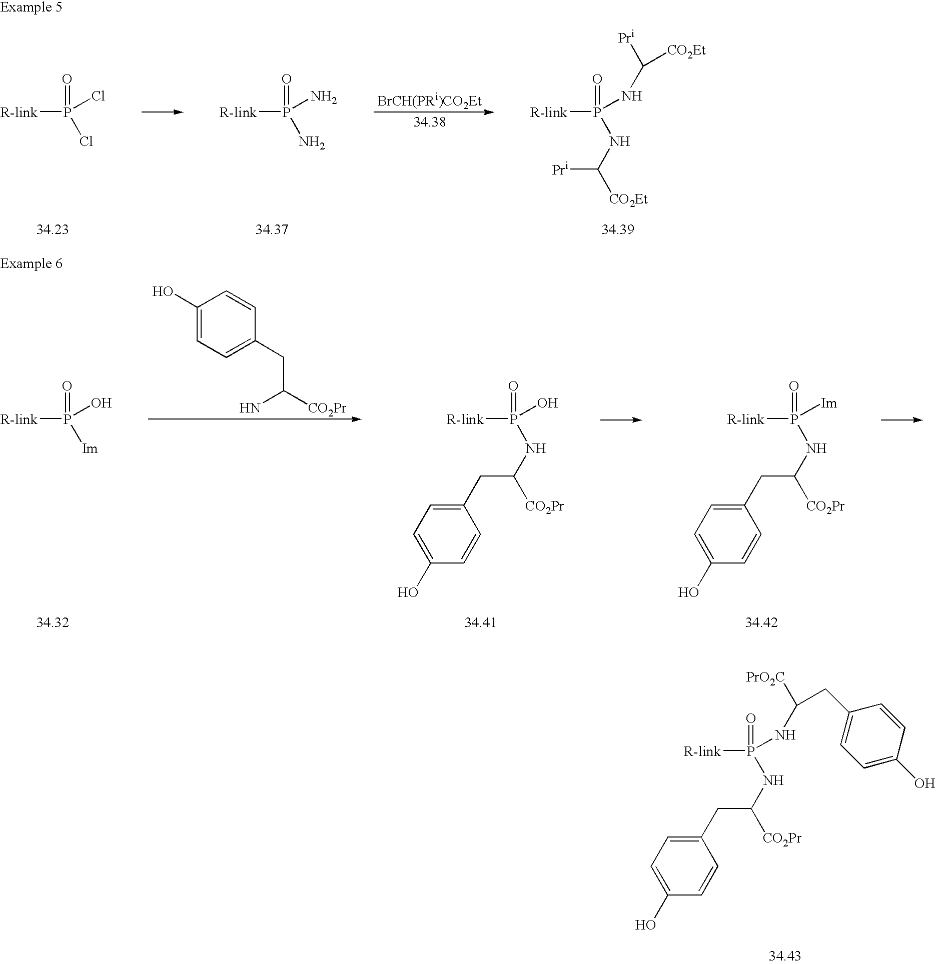

Examples of this method are shown in Scheme 35, Examples and 2 and 3. In the sequence shown in Example 2, a monobenzyl phosphonate 35.11 is transformed by reaction with ethyl alaninate, using one of the methods described above, into the monoamidate 35.12. The benzyl group is then removed by catalytic hydrogenation in ethylacetate solution over a 5% palladium on carbon catalyst, to afford the phosphonic acid amidate 35.13. The product is then reacted in dichloromethane solution at ambient temperature with equimolar amounts of 1-(dimethylaminopropyl)-3-ethylcarbodiimide and trifluoroethanol 35.14, for example as described in Tet. Lett., 2001, 42, 8841, to yield the amidate ester 35.15.

In the sequence shown in Scheme 35, Example 3, the monoamidate 35.13 is coupled, in tetrahydrofuran solution at ambient temperature, with equimolar amounts of dicyclohexyl carbodiimide and 4-hydroxy-N-methylpiperidine 35.16, to produce the amidate ester product 35.17.

Using the above procedures, but employing, in place of the ethyl alaninate product 35.12 different monoacids 35.2, and in place of trifluoroethanol 35.14 or 4-hydroxy-N-methylpiperidine 35.16, different hydroxy compounds R³OH, the corresponding products 35.3 are obtained.

Alternatively, the activated phosphonate ester 34.8 is reacted with ammonia to yield the amidate 35.4. The product is then reacted, as described in Scheme 34, with a haloester 35.5, in the presence of a base, to produce the amidate product 35.6. If appropriate, the nature of the R¹ group is changed, using the procedures described above, to give the product 35.3. The method is illustrated in Scheme 35, Example 4. In this sequence, the monophenyl phosphoryl chloride 35.18 is reacted, as described in Scheme 34, with ammonia, to yield the amino product 35.19. This material is then reacted in N-methylpyrrolidinone solution at 170° with butyl 2-bromo-3-phenylpropionate 35.20 and potassium carbonate, to afford the amidate product 35.21.

Using these procedures, but employing, in place of butyl 2-bromo-3-phenylpropionate 35.20, different haloesters 35.5, the corresponding products 35.6 are obtained.

The monoamidate products 35.3 are also prepared from a the doubly activated phosphonate derivatives 34.7. In this procedure, examples of which are described in Synlett., 1998, 1, 73, the intermediate 34.7 is reacted with a limited amount of the aminoester 34.9 to give the mono-displacement product 34.11. The latter compound is then reacted with the hydroxy compound R³OH in a polar organic solvent such as dimethylformamide, in the presence of a base such as diisopropylethylamine, to yield the monoamidate ester 35.3.

The method is illustrated in Scheme 35, Example 5. In this method, the phosphoryl dichloride 35.22 is reacted in dichloromethane solution with one molar equivalent of ethyl N-methyl tyrosinate 35.23 and dimethylaminopyridine, to generate the monoamidate 35.24. The product is then reacted with phenol 35.25 in dimethylformamide containing potassium carbonate, to yield the ester amidate product 35.26.

Using these procedures, but employing, in place of ethyl N-methyl tyrosinate 35.23 or phenol 35.25, the aminoesters 34.9 and/or the hydroxy compounds R³OH, the corresponding products 35.3 are obtained.

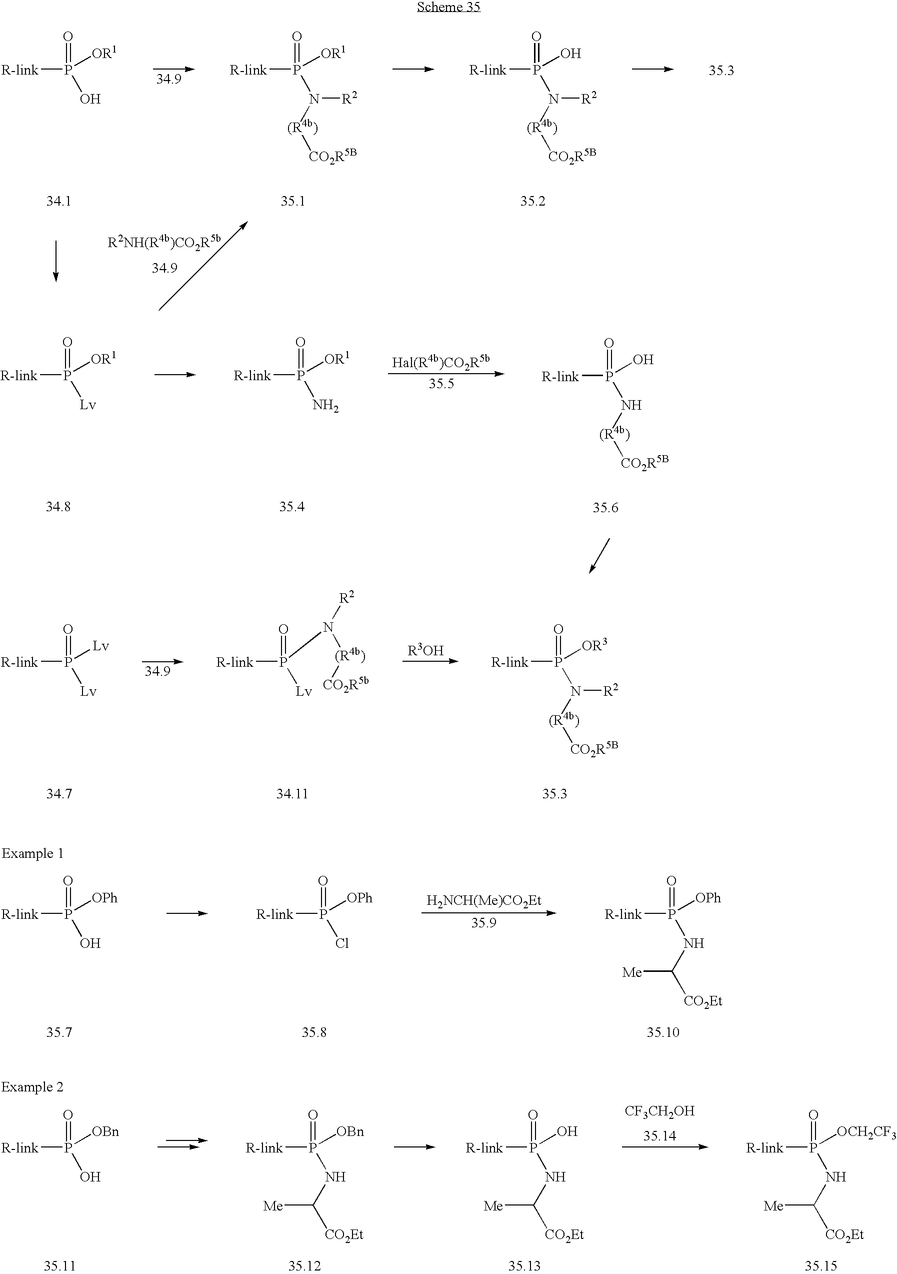

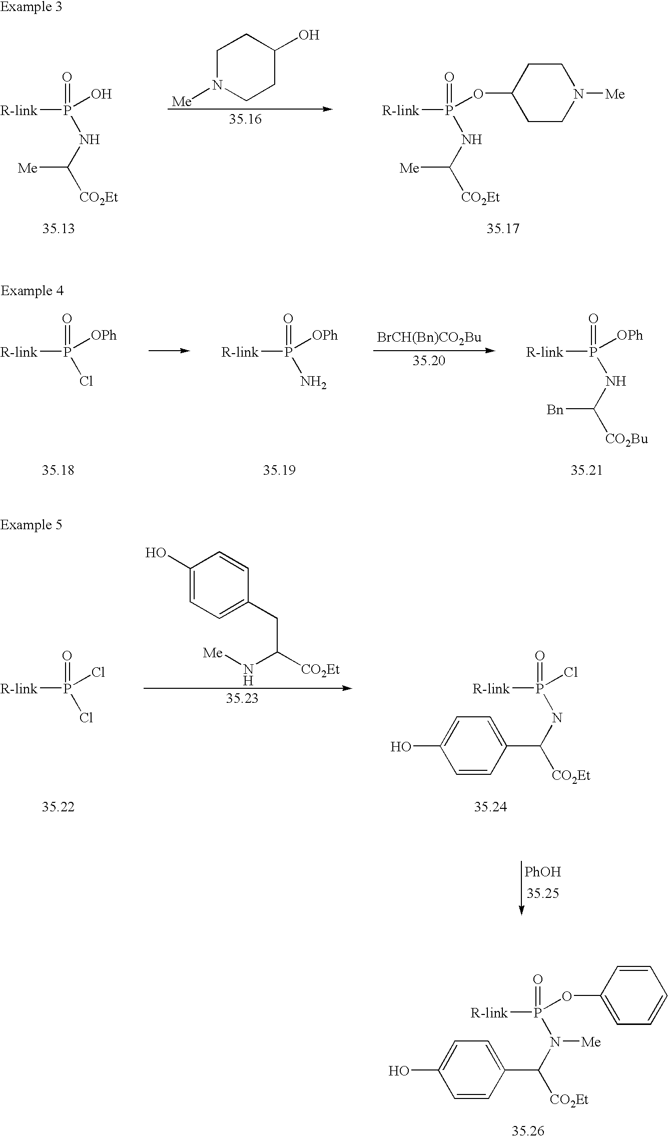

Scheme 36 illustrates methods for the preparation of carboalkoxy-substituted phosphonate diesters in which one of the ester groups incorporates a carboalkoxy substituent.

In one procedure, a phosphonate monoester 34.1, prepared as described above, is coupled, using one of the methods described above, with a hydroxyester 36.1, in which the groups R^{4b} and R^{5b} are as described in Scheme 34. For example, equimolar amounts of the reactants are coupled in the presence of a carbodiimide such as dicyclohexyl carbodiimide, as described in Aust. J. Chem., 1963, 609, optionally in the presence of dimethylaminopyridine, as described in Tet., 1999, 55, 12997. The reaction is conducted in an inert solvent at ambient temperature.

The procedure is illustrated in Scheme 36, Example 1. In this method, a monophenyl phosphonate 36.9 is coupled, in dichloromethane solution in the presence of dicyclohexyl carbodiimide, with ethyl 3-hydroxy-2-methylpropionate 36.10 to yield the phosphonate mixed diester 36.11.

Using this procedure, but employing, in place of ethyl 3-hydroxy-2-methylpropionate 36.10, different hydroxyesters 33.1, the corresponding products 33.2 are obtained.

The conversion of a phosphonate monoester 34.1 into a mixed diester 36.2 is also accomplished by means of a Mitsunobu coupling reaction with the hydroxyester 36.1, as described in Org. Lett., 2001, 643. In this method, the reactants 34.1 and 36.1 are combined in a polar solvent such as tetrahydrofuran, in the presence of a triarylphosphine and a dialkyl azodicarboxylate, to give the mixed diester 36.2. The R¹ substituent is varied by cleavage, using the methods described previously, to afford the monoacid product 36.3. The product is then coupled, for example using methods described above, with the hydroxy compound R³OH, to give the diester product 36.4.

The procedure is illustrated in Scheme 36, Example 2. In this method, a monoallyl phosphonate 36.12 is coupled in tetrahydrofuran solution, in the presence of triphenylphosphine and diethylazodicarboxylate, with ethyl lactate 36.13 to give the mixed diester 36.14. The product is reacted with tris(triphenylphosphine) rhodium chloride (Wilkinson catalyst) in acetonitrile, as described previously, to remove the allyl group and produce the monoacid product 36.15. The latter compound is then coupled, in pyridine solution at ambient temperature, in the presence of dicyclohexyl carbodiimide, with one molar equivalent of 3-hydroxypyridine 36.16 to yield the mixed diester 36.17.

Using the above procedures, but employing, in place of the ethyl lactate 36.13 or 3-hydroxypyridine, a different hydroxyester 36.1 and/or a different hydroxy compound R³OH, the corresponding products 36.4 are obtained.

The mixed diesters 36.2 are also obtained from the monoesters 34.1 via the intermediacy of the activated monoesters 36.5. In this procedure, the monoester 34.1 is converted into the activated compound 36.5 by reaction with, for example, phosphorus pentachloride, as described in J. Org. Chem., 2001, 66, 329, or with thionyl chloride or oxalyl chloride (Lv=Cl), or with triisopropylbenzenesulfonyl chloride in pyridine, as described in Nucleosides and Nucleotides, 2000, 19, 1885, or with carbonyl diimidazole, as described in J. Med. Chem., 2002, 45, 1284. The resultant activated monoester is then reacted with the hydroxyester 36.1, as described above, to yield the mixed diester 36.2.

The procedure is illustrated in Scheme 36, Example 3. In this sequence, a monophenyl phosphonate 36.9 is reacted, in acetonitrile solution at 70° C., with ten equivalents of thionyl chlorides so as to produce the phosphoryl chloride 36.19. The product is then reacted with ethyl 4-carbamoyl-2-hydroxybutyrate 36.20 in dichloromethane containing triethylamine, to give the mixed diester 36.21.

Using the above procedures, but employing, in place of ethyl 4-carbamoyl-2-hydroxybutyrate 36.20, different hydroxyesters 36.1, the corresponding products 36.2 are obtained.

The mixed phosphonate diesters are also obtained by an alternative route for incorporation of the R³O group into intermediates 36.3 in which the hydroxyester moiety is already incorporated. In this procedure, the monoacid intermediate 36.3 is converted into the activated derivative 36.6 in which Lv is a leaving group such as chloro, imidazole, and the like, as previously described. The activated intermediate is then reacted with the hydroxy compound R³OH, in the presence of a base, to yield the mixed diester product 36.4.

The method is illustrated in Scheme 36, Example 4. In this sequence, the phosphonate monoacid 36.22 is reacted with trichloromethanesulfonyl chloride in tetrahydrofuran containing collidine, as described in J. Med. Chem., 1995, 38, 4648, to produce the trichloromethanesulfonyloxy product 36.23. This compound is reacted with 3-(morpholinomethyl)phenol 36.24 in dichloromethane containing triethylamine, to yield the mixed diester product 36.25.

Using the above procedures, but employing, in place of with 3-(morpholinomethyl)phenol 36.24, different carbinols R³OH, the corresponding products 36.4 are obtained.

The phosphonate esters 36.4 are also obtained by means of allylation reactions performed on the monoesters 34.1. The reaction between the monoacid 34.1 and the haloester 36.7 is performed in a polar solvent in the presence of a base such as diisopropylethylamine, as described in Anal. Chem., 1987, 59, 1056, or triethylamine, as described in J. Med. Chem., 1995, 38, 1372, or in a non-polar solvent such as benzene, in the presence of 18-crown-6, as described in Syn. Comm., 1995, 25, 3565.

The method is illustrated in Scheme 36, Example 5. In this procedure, the monoacid 36.26 is reacted with ethyl 2-bromo-3-phenylpropionate 36.27 and diisopropylethylamine in dimethylformamide at 80° C. to afford the mixed diester product 36.28.

Using the above procedure, but employing, in place of ethyl 2-bromo-3-phenylpropionate 36.27, different haloesters 36.7, the corresponding products 36.4 are obtained.

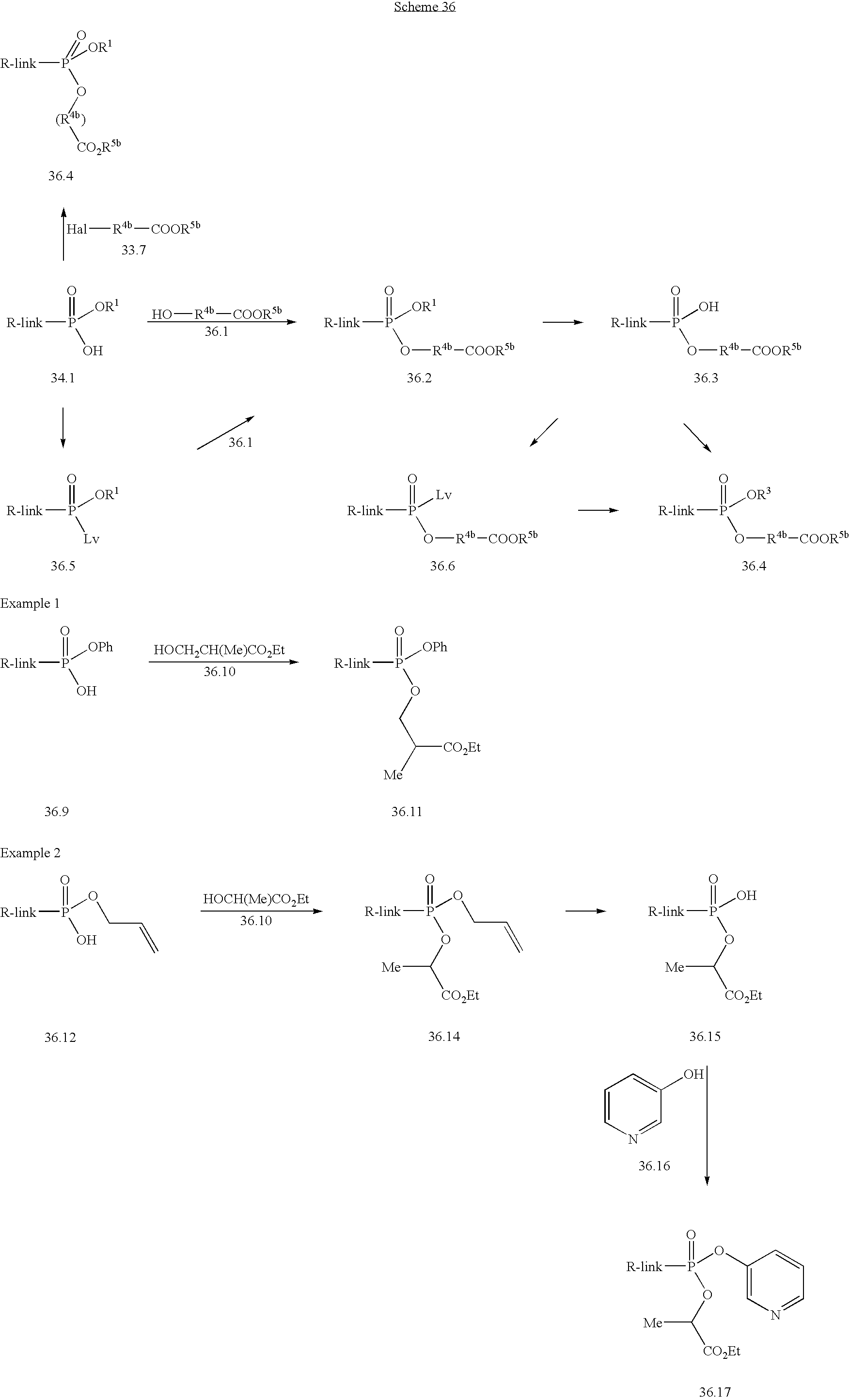

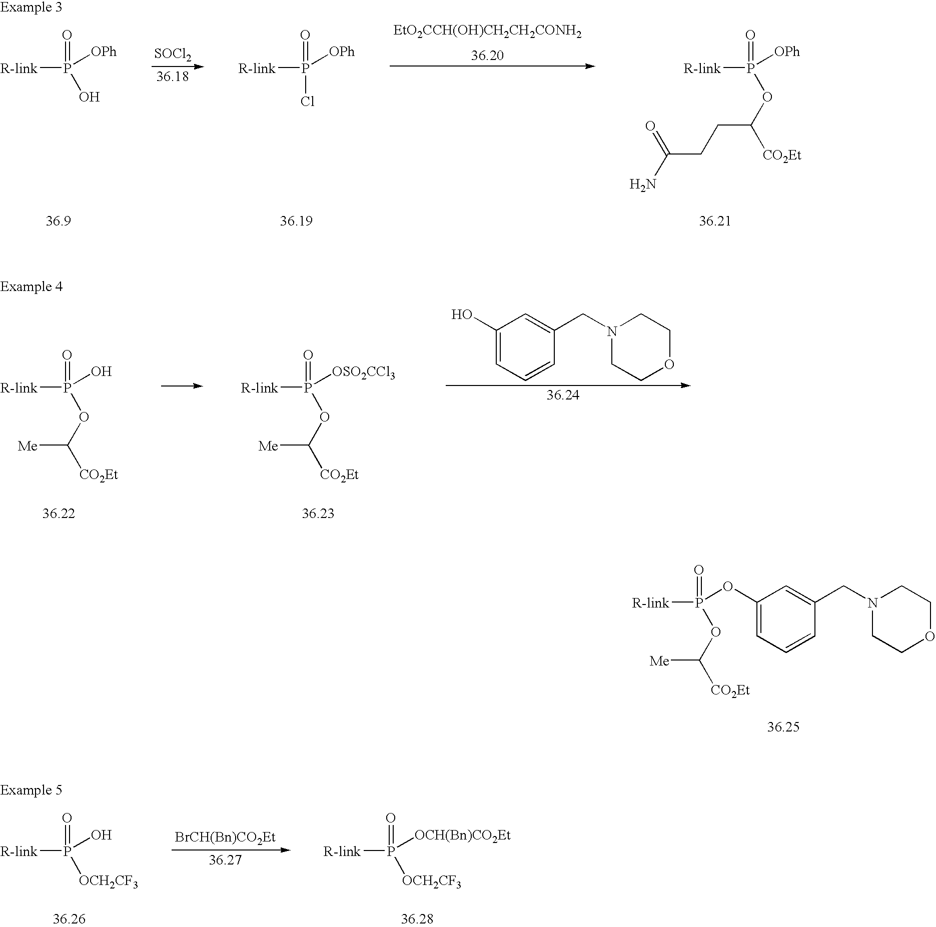

Scheme 37 illustrates methods for the preparation of phosphonate diesters in which both the ester substituents incorporate carboalkoxy groups.

The compounds are prepared directly or indirectly from the phosphonic acids 34.6. In one alternative, the phosphonic acid is coupled with the hydroxyester 37.2, using the conditions described previously in Schemes 34-36, such as coupling reactions using dicyclohexyl carbodiimide or similar reagents, or under the conditions of the Mitsunobu reaction, to afford the diester product 37.3 in which the ester substituents are identical.

This method is illustrated in Scheme 37, Example 1. In this procedure, the phosphonic acid 34.6 is reacted with three molar equivalents of butyl lactate 37.5 in the presence of Aldrithiol-2 and triphenyl phosphine in pyridine at ca. 70° C., to afford the diester 37.6.

Using the above procedure, but employing, in place of butyl lactate 37.5, different hydroxyesters 37.2, the corresponding products 37.3 are obtained.

Alternatively, the diesters 37.3 are obtained by alkylation of the phosphonic acid 34.6 with a haloester 37.1. The alkylation reaction is performed as described in Scheme 36 for the preparation of the esters 36.4.

This method is illustrated in Scheme 37, Example 2. In this procedure, the phosphonic acid 34.6 is reacted with excess ethyl 3-bromo-2-methylpropionate 37.7 and diisopropylethylamine in dimethylformamide at ca. 80° C., as described in Anal. Chem., 1987, 59, 1056, to produce the diester 37.8.

Using the above procedure, but employing, in place of ethyl 3-bromo-2-methylpropionate 37.7, different haloesters 37.1, the corresponding products 37.3 are obtained.

The diesters 37.3 are also obtained by displacement reactions of activated derivatives 34.7 of the phosphonic acid with the hydroxyesters 37.2. The displacement reaction is performed in a polar solvent in the presence of a suitable base, as described in Scheme 36. The displacement reaction is performed in the presence of an excess of the hydroxyester, to afford the diester product 37.3 in which the ester substituents are identical, or sequentially with limited amounts of different hydroxyesters, to prepare diesters 37.3 in which the ester substituents are different.

The methods are illustrated in Scheme 37, Examples 3 and 4. As shown in Example 3, the phosphoryl dichloride 35.22 is reacted with three molar equivalents of ethyl 3-hydroxy-2-(hydroxymethyl)propionate 37.9 in tetrahydrofuran containing potassium carbonate, to obtain the diester product 37.10.

Using the above procedure, but employing, in place of ethyl 3-hydroxy-2-(hydroxymethyl)propionate 37.9, different hydroxyesters 37.2, the corresponding products 37.3 are obtained.

Scheme 37, Example 4 depicts the displacement reaction between equimolar amounts of the phosphoryl dichloride 35.22 and ethyl 2-methyl-3-hydroxypropionate 37.11, to yield the monoester product 37.12. The reaction is conducted in acetonitrile at 70° in the presence of diisopropylethylamine. The product 37.12 is then reacted, under the same conditions, with one molar equivalent of ethyl lactate 37.13, to give the diester product 37.14.

Using the above procedures, but employing, in place of ethyl 2-methyl-3-hydroxypropionate 37.11 and ethyl lactate 37.13, sequential reactions with different hydroxyesters 37.2, the corresponding products 37.3 are obtained.

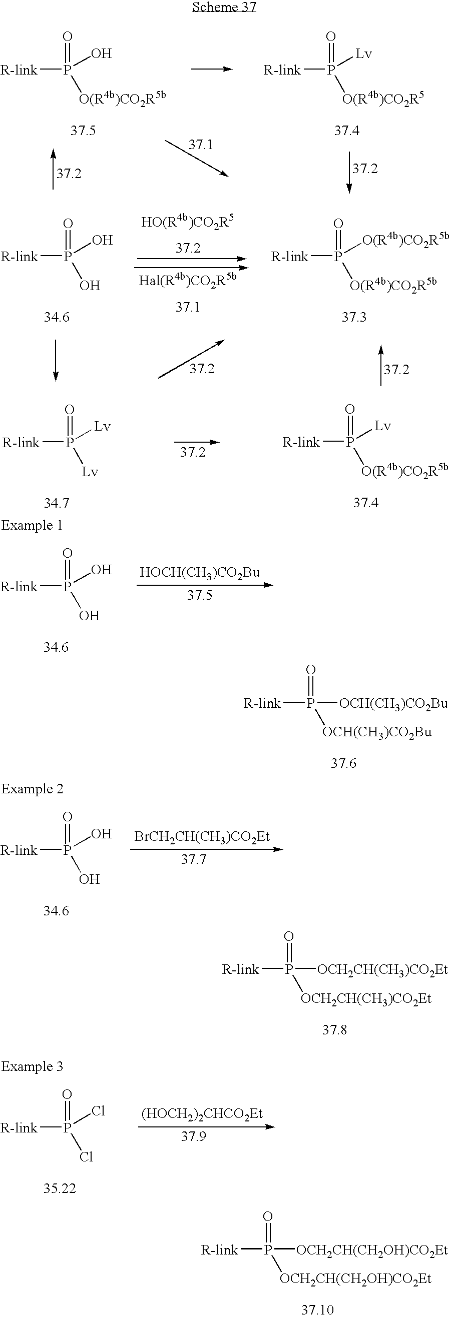

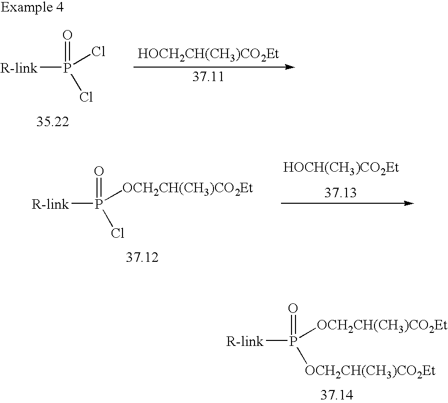

2,2-Dimethyl-2-aminoethylphosphonic acid intermediates can be prepared by the route in Scheme 5. Condensation of 2-methyl-2-propanesulfinamide with acetone give sulfinyl imine 38.11 (J. Org. Chem. 1999, 64, 12). Addition of dimethyl methylphosphonate lithium to 38.11 afford 38.12. Acidic methanolysis of 38.12 provide amine 38.13. Protection of amine with Cbz group and removal of methyl groups yield phosphonic acid 38.14, which can be converted to desired 38.15 (Scheme 5a) using methods reported earlier on. An alternative synthesis of compound 38.14 is also shown in Scheme 5b. Commercially available 2-amino-2-methyl-1-propanol is converted to aziridines 38.16 according to literature methods (J. Org. Chem. 1992, 57, 5813; Syn. Lett. 1997, 8, 893). Aziridine opening with phosphite give 38.17 (Tetrahedron Lett. 1980, 21, 1623). Reprotection) of 38.17 affords 38.14.

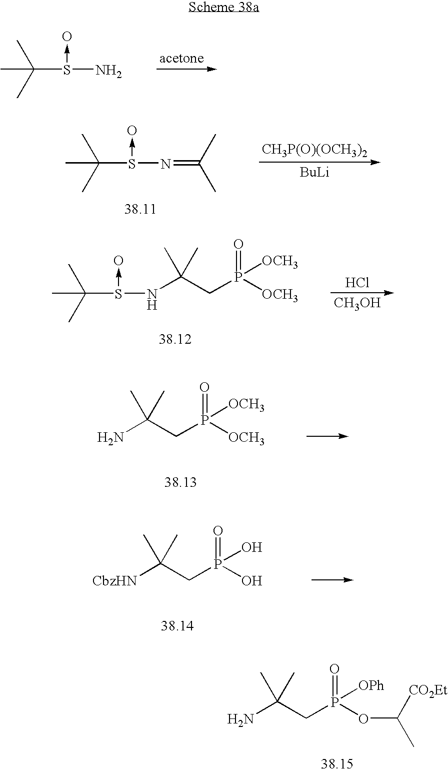

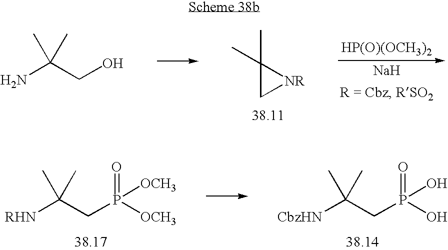

### Biological Activity of HIV-Integrase Inhibitor Compounds

Representative compounds of the invention are tested for biological activity by methods including anti-HIV assay, measuring inhibition of HIV-integrase strand transfer catalysis, and cytotoxicity. See: Wolfe, etal J. Virol. (1996) 70:1424-1432; Hazuda, etal Nucleic Acids Res. (1994) 22:1121-22; Hazuda, etal J. Virol. (1997) 71:7005-7011; Hazuda, etal Drug Design and Discovery (1997) 15:17-24; and Hazuda, etal Science (2000) 287:646-650. The antiviral activity of a compound of the invention can be determined using pharmacological models which are well known in the art. While many of the compounds of the present invention demonstrate inhibition of integration of HIV reverse-transcribed DNA, there may be other mechanisms of action whereby HIV replication or proliferation is affected. The compounds of the invention may be active via inhibition of HIV-integrase or other enzymes associated with HIV infection, AIDS, or ARC. Furthermore, the compounds of the invention may have significant activity against other viral diseases. Thus, the specific assays embodied in Examples x-y are not meant to limit the present invention to a specific mechanism of action.

### Pharmaceutical Formulations and Routes of Administration

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the Handbook of Pharmaceutical Excipients (1986) and include ascorbic acid and other antioxidants, chelating agents such as EDTA, carbohydrates such as dextrin, hydroxyalkylcellulose, hydroxyallylmethylcellulose, stearic acid and the like.

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferably to present them as pharmaceutical formulations. The formulations, both for veterinary and for human use, of the present invention comprise at least one active ingredient, as above defined, together with one or more pharmaceutically acceptable carriers therefor and optionally other. therapeutic ingredients. The carrier(s) must be “acceptable” in the sense of being compatible with the other ingredients of the formulation and not deleterious to the recipient thereof.

The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the present invention include Tween™ 60, Span™ 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents such as pentamidine for treatment of pneumocystis pneumonia.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

The present invention further provides veterinary compositions comprising at least one active ingredient as above defined together with a veterinary carrier therefor. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Controlled release formulations may be employed for the treatment or prophylaxis of various microbial infections particularly human bacterial, human parasitic protozoan or human viral infections caused by microbial species including Plasmodium, Pneumocystis, herpes viruses (CMV, HSV 1, HSV 2, VZV, and the like), retroviruses, adenoviruses and the like. The controlled release formulations can be used to treat HIV infections and related conditions such as tuberculosis, malaria, pneumocystis pneumonia, CMV retinitis, AIDS, AIDS-related complex (ARC) and progressive generalized lymphadeopathy (PGL), and AIDS-related neurological conditions such as multiple sclerosis, and tropical spastic paraparesis. Other human retroviral infections that may be treated with the controlled release formulations according to the invention include Human T-cell Lymphotropic virus (HTLV)-I and IV and HIV-2 infections. The invention accordingly provides pharmaceutical formulations for use in the treatment or prophylaxis of the above-mentioned human or veterinary conditions and microbial infections.

### Combination Therapy

The compounds of the invention may be employed in combination with other therapeutic agents for the treatment or prophylaxis of the infections or conditions indicated above. Examples of such further therapeutic agents include agents that are effective for the treatment or prophylaxis of viral, parasitic or bacterial infections or associated conditions or for treatment of tumors or related conditions include 3′-azido-3′-deoxythymidine (zidovudine, AZT), 2′-deoxy-3′-thiacytidine (3TC), 2′,3′-dideoxy-2′,3′-didehydroadenosine (D4A), 2′,3′-dideoxy-2′,3′-didehydrothymidine (D4T), carbovir (carbocyclic 2′,3′-dideoxy-2′,3′-didehydroguanosine), 3′-azido-2′,3′-dideoxyuridine, 5-fluorothymidine, (E)-5-(2-bromovinyl)-2′-deoxyuridine (BVDU), 2-chlorodeoxyadenosine, 2-deoxycoformycin, 5-fluorouracil, 5-fluorouridine, 5-fluoro-2′-deoxyuridine, 5-trifluoromethyl-2′-deoxyuridine, 6-azauridine, 5-fluoroorotic acid, methotrexate, triacetyluridine, 1-(2′-deoxy-2′-fluoro-1-β-arabinosyl)-5-iodocytidine (FIAC), tetrahydro-imidazo(4,5,1 jk)-(1,4)-benzodiazepin-2(1H)-thione (TIBO), 2′-nor-cyclicGMP, 6-methoxypurine arabinoside (ara-M), 6-methoxypurine arabinoside 2′-O-valerate, cytosine arabinoside (ara-C), 2′,3′-dideoxynucleosides such as 2′,3′-dideoxycytidine (ddC), 2′,3′-dideoxyadenosine (ddA) and 2′,3′-dideoxyinosine (ddI), acyclic nucleosides such as acyclovir, penciclovir, famciclovir, ganciclovir, HPMPC, PMEA, PMEG, PMPA, PMPDAP, FPMPA, HPMPA, HPMPDAP, (2R,5R)-9-<tetrahydro-5-(phosphonomethoxy)-2-furanyladenine, (2R,5R)-1-<tetrahydro-<5-(phosphonomethoxy)-2-furanylthymine, other antivirals including ribavirin (adenine arabinoside), 2-thio-6-azauridine, tubercidin, aurintricarboxylic acid, 3-deazaneoplanocin, neoplanocin, rimantidine, adamantine, and foscarnet (trisodium phosphonoformate), antibacterial agents including bactericidal fluoroquinolones (ciprofloxacin, pefloxacin and the like), aminoglycoside bactericidal antibiotics (streptomycin, gentamicin, amicacin and the like) β-lactamase inhibitors (cephalosporins, penicillins and the like), other antibacterials including tetracycline, isoniazid, rifampin, cefoperazone, claithromycin and azithromycin, antiparasite or antifungal agents including pentamidine (1,5-bis(4′-aminophenoxy)pentane), 9-deaza-inosine, sulfamethoxazole, sulfadiazine, quinapyramine, quinine, fluconazole, ketoconazole, itraconazole, Amphotericin B, 5-fluorocytosine, clotrimazole, hexadecylphosphocholine and nystatin, renal excretion inhibitors such as probenicid, nucleoside transport inhibitors such as dipyridamole, dilazep and nitrobenzylthioinosine, immunomodulators such as FK506, cyclosporin A, thymosin α-1, cytokines including TNF and TGF-β, interferons including IFN-α, IFN-β, and IFN-γ, interleukins including various interleukins, macrophage/granulocyte colony stimulating factors including GM-CSF, G-CSF, M-CSF, cytolcine antagonists including anti-TNF antibodies, anti-interleukin antibodies, soluble interleukin receptors, protein kinase C inhibitors and the like.

The invention includes a pharmaceutical composition comprising a therapeutically effective amount of a Formula I or II compound in combination with a therapeutically effective amount of an AIDS treatment agent selected from:

(1) an AIDS antiviral agent,

(2) an anti-infective agent, and

(3) an immunomodulator.

It is also possible to combine any of the compounds of the invention in a unitary dosage form for simultaneous administration with a second, or third, active pharmaceutical ingredient. The two or three-part combination may also be administered sequentially in two or three administrations. Second and third active ingredients may have anti-HIV activity and include protease inhibitors (Prt), nucleoside reverse transcriptase inhibitors (NRTI), non-nucleoside reverse transcriptase inhibitors (NNRTI), and integrase inhibitors. Exemplary second and third active anti-HIV ingredients to be administered in combination with the compounds of the invention, i.e. Formulas I and II compounds, are:
5,6 dihydro-5-azacytidine
5-aza 2′deoxycytidine
5-azacytidine
5-yl-carbocyclic 2′-deoxyguanosine (BMS200,475)
9 (arabinofuranosyl)guanine; 9-(2′ deoxyribofuranosyl)guanine
9-(2′-deoxy 2′fluororibofuranosyl)-2,6-diaminopurine
9-(2′-deoxy 2′fluororibofuranosyl)guanine
9-(2′-deoxyribofuranosyl)-2,6 diaminopurine
9-(arabinofuranosyl)-2,6 diaminopurine
Abacavir, Ziagen®
Acyclovir, ACV; 9-(2-hydroxyethoxylmethyl)guanine
Adefovir dipivoxil, Hepsera®
amdoxivir, DAPD
Amprenavir, Agenerase®
araA; 9-b-D-arabinofuranosyladenine (Vidarabine)
AZT; 3′-azido-2′,3′-dideoxythymdine, Zidovudine, (Retrovir®)
BHCG; (.+−.)-(1a,2b,3a)-9-[2,3-bis(hydroxymethyl)cyclobutyl]guanine
BMS200,475; 5-yl-carbocyclic 2′-deoxyguanosine
Buciclovir; (R) 9-(3,4-dihydroxybutyl)guanine
BvaraU; 1-b-D-arabinofuranosyl-E-5-(2-bromovinyl)uracil (Sorivudine)
Calanolide A
Capravirine
CDG; carbocyclic 2′-deoxyguanosine
Cidofovir, HPMPC; (S)-9-(3-hydroxy-2-phosphonylmethoxypropyl)cytosine
Clevudine, L-FMAU; 2′-Fluoro-5-methyl-p-L-arabino-furanosyluracil
Cytallene; [1-(4′-hydroxy-1′,2′-butadienyl)cytosine]
d4C; 3′-deoxy-2′,3′-didehydrocytidine
DAPD; (−)-β-D-2,6-diaminopurine dioxolane
ddA; 2′,3′-dideoxyadenosine
ddAPR; 2,6-diaminopurine-2′,3′-dideoxyriboside
ddC; 2′,3′-dideoxycytidine (Zalcitabine)
ddI; 2′,3′-dideoxyinosine, didanosine, (Videx®)
Delavirdine, Rescriptor®
Didanosine, ddI, Videx®; 2′,3′-dideoxyinosine
DXG; dioxolane guanosine
E-5-(2-bromovinyl)-2′-deoxyuridine
Efavirenz, Sustiva®
Enfuvirtide, Fuzeon®
P F-ara-A; fluoroarabinosyladenosine (Fludarabine)
FDOC; (−)-β-D-5-fluoro-1-[2-(hydroxymethyl)-1,3-dioxolane]cytosine
FEAU; 2′-deoxy-2′-fluoro-1-β-D-arabinofuranosyl-5-ethyluracil
FIAC; 1-(2-deoxy-2-fluoro-β-D-arabinofuranosyl)-5-iodocytosine
FIAU; 1-(2-deoxy-2-fluoro-β-D-arabinofuranosyl)-5-iodouridine
FLG; 2′,3′-dideoxy-3′-fluoroguanosine
FLT; 3′-deoxy-3′-fluorothymidine
Fludarabine; F-ara-A; fluoroarabinosyladenosine
FMAU; 2′-Fluoro-5-methyl-b-L-arabino-furanosyluracil
FMdC
Foscarnet; phosphonoformic acid, PFA
FPMPA; 9-(3-fluoro-2-phosphonylmethoxypropyl)adenine
Gancyclovir, GCV; 9-(1,3-dihydroxy-2-propoxymethyl)guanine
GS-7340; 9-[R-2-[[(S)-[[(S)-1-(isopropoxycarbonyl)ethyl]amino]-phenoxyphosphinyl]methoxy]propyl]adenine
HPMPA; (S)-9-(3-hydroxy-2-phosphonylmethoxypropyl)adenine
HPMPC; (S)-9-(3-hydroxy-2-phosphonylmethoxypropyl)cytosine (Cidofovir)
Hydroxyurea, Droxia®
Indinavir, Crixivan®
Lamivudine, 3TC, Epivir™; (2R,5S, cis)-4-amino-1-(2-hydroxymethyl-1,3-oxathiolan-5-yl)-(1H)-pyrimidin-2-one
L-d4C; L-3′-deoxy-2′,3′-didehydrocytidine
L-ddC; L-2′,3′-dideoxycytidine
L-Fd4C; L-3′-deoxy-2′,3′-didehydro-5-fluorocytidine
L-FddC; L-2′,3′-dideoxy-5-fluorocytidine
Lopinavir
Nelfinavir, Viracept®
Nevirapine, Viramune®
Oxetanocin A; 9-(2-deoxy-2-hydroxymethyl-beta-D-erythro-oxetanosyl)adenine
Oxetanocin G; 9-(2-deoxy-2-hydroxymethyl-β-D-erythro-oxetanosyl)guanine
Penciclovir
PMEDAP; 9-(2-phosphonylmethoxyethyl)-2,6-diaminopurine
PMPA, tenofovir; (R)-9-(2-phosphonylmethoxypropyl)adenine
PPA; phosphonoacetic acid
Ribavirin; 1-β-D-ribofuranosyl-1,2,4-triazole-3-carboxamide
Ritonavir, Norvir®
Saquinavir, Invirase®, Fortovase®
Sorivudine, Bvara U; 1-β-D-arabinofuranosyl-E-5-(2-bromovinyl)uracil
Stavudine, d4T, Zerit®; 2′,3′-didehydro-3′-deoxythymidine
Trifluorothymidine, TFT; Trifluorothymidine
Vidarabine, araA; 9-O-D-arabinofuranosyladenine
Zalcitabine, Hivid®, ddC; 2′,3′-dideoxycytidine
Zidovudine, AZT, Retrovir®; 3′-azido-2′,3′-dideoxythymdine
Zonavir; 5-propynyl-1-arabinosyluracil

### Assay Protocol Examples

### HIV Integrase Assay (IC₅₀ Determination)

IC50 (also referred to as CC50, CD50, TC50, TD50 or cytotoxicity) is the inhibitory concentration that reduces cellular growth or viability of uninfected cells by 50%.

HIV Integrase assay is carried out in Reacti-Bind High Binding Capacity Streptavidin coated plates (Pierce # 15502) in 100 μl reactions. The wells of the plate are rinsed once with PBS. Each well is then coated at room temperature for 1 h with 100 μl of 0.14 μM double-stranded, 5′-biotin labelled donor DNA.

After coating, the plate is washed twice with PBS. 3′ Processing of the donor DNA is started by adding 80 μl of Integrase/buffer mixture (25 mM HEPES, pH 7.3, 12.5 mM DTT, 93.75 mM NaCl, 12.5 mM MgCl₂, 1.25% Glycerol, 0.3125 μM integrase) to each well. 3′-Processing is allowed to proceed for 30 min at 37° C., after which, 10 μl of test compound and 101 of 2.5 μM 3′-DIG (digitoxigenin)-labeled, double-stranded Target DNA are added to each well to allow strand transfer to proceed for 30 min at 37° C. The plate is then washed three times with 2×SSC for 5 min and rinsed once with PBS. For detection of integrated product, 100 μl of a 1/2000 dilution of HRP-conjugated anti-DIG antibody (Pierce #31468) are added to each well and incubated for 1 hour. The plate is then washed three times for 5 min each, with 0.05% Tween-20 in PBS. For signal development and amplification, 100 μl of SuperSignal ELISA Femto Substrate (Pierce #37075) are added to each well. Chemiluminescence (in relative light units) is read immediately at 425 nm in the SPECTRAmax GEMINI Microplate Spectrophotometer using the end point mode at 5 sec per well. For IC₅₀ determinations, eight concentrations of test compounds in a 1/2.2 dilution series are used. Certain compounds of the invention, including those in Tables 1-5, had a strand transfer IC₅₀ less than about 10 μM.

### Anti-HIV Assay (EC₅₀ Determination)

EC50 (also commonly referred to as ED50 or IC50) is the effective concentration that inhibits 50% of viral production, 50% of viral infectivity, or 50% of the virus-induced cytopathic effect.

Anti-HIV assay is carried out in 96-well Clear Bottom Black Assay Plate (Costar # 3603) in 100 μl of culture medium, using the CellTiter-Glo™ Reagent (Promega # G7570) for signal detection. MT-2 cells (1.54×10⁴ cells) are infected with wild-type virus at an m.o.i. (multiplicity of infection, i.e. the ratio between the number of infectious viral particles and cells in an assay) of about 0.025, and grown in the presence of various drug concentrations (serial 5-fold dilutions) in 100 μl of RPMI medium containing 10% FBS, 2% glutamine, 1% HEPES and 1% penicillin/streptomycin for 5 days. At the end of the incubation period, 100 μl of CellTiter-Glo™ Reagent is added to each well in the Assay Plate and the chemiluminescence (in relative light units) is measured after 10 mins of incubation with the Wallac Victor² 1420 MultiLabel Counter. Certain compounds of the invention, including those in Tables 1-5, had an anti-HIV MT2 EC₅₀ less than about 10 μM.

### Cytotoxicity Assay (CC₅₀ Determination)

For the determination of compound cytotoxicity, the plate and reagents are the same as those of anti-HIV assay. Uninfected MT-2 cells (1.54×10⁴ cells) are grown in the presence of various drug concentrations (serial 2-fold dilutions) in 100 μl of RPMI medium containing 10% FBS, 2% glutamine, 1% HEPES and 1% penicillin/streptomycin for 5 days. At the end of the incubation period, 100 μl of CellTiter-Glo™ Reagent is added to each well in the assay plate and the chemiluminescence (in relative light units) is measured after 10 mins of incubation with the Wallac Victor² 1420 MultiLabel Counter.

What is claimed:

1. A compound selected from Formulas I and II: 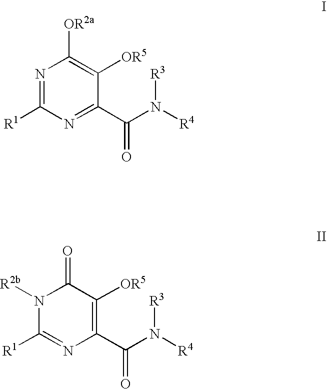
or a pharmaceutically acceptable salt thereof, and including all enol, tautomeric, and resonance isomers, enantiomers, diastereomers, and racemic mixtures thereof;
wherein:
R¹ is selected from H, F, Cl, Br, I, OH, OR, amino (—NH₂), ammonium (—NH₃⁺), alkylamino (—NHR), dialkylamino (—NR₂), trialkylammonium (—NR₃⁺), carboxyl (—CO₂H), sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), allylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ allyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, L-A³, and a prodrug moiety;
R^{2a} and R⁵ are each independently selected from H, carboxyl (—CO₂H), sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ alkyl, C₁-C₁₈ substituted allyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, L-A³, and a prodrug moiety;
R^{2b}, R³, and R⁴ are each independently selected from H, OH, OR, amino (—NH₂), ammonium (—NH₃⁺), alkylamino (—NHR), dialkylamino (—NR₂), trialkylammonium (—NR₃⁺), carboxyl (—CO₂H), sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, and C₂-C₂₀ substituted heterocycle phosphonate, phosphate, polyethyleneoxy, a protecting group, L-A³, and a prodrug moiety;
R is independently selected from H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, C₂-C₂₀ substituted heterocycle, phosphonate, phosphate, polyethyleneoxy, a protecting group, and a prodrug moiety;
L is selected from a bond, O, S, NR, N—OR, C₁-C₁₂ alkylene, C₁-C₁₂ substituted allylene, C₂-C₁₂ alkenylene, C₂-C₁₂ substituted alkenylene, C₂-C₁₂ alkynylene, C₂-C₁₂ substituted alkynylene, C₆-C₂₀ arylene, C₆-C₂₀ substituted arylene, C(═O)NH, C(═O), S(═O)₂, C(═O)NH(CH₂)ₙ, and (CH₂CH₂O)ₙ, where n may be 1, 2, 3, 4, 5, or 6;
A³ has the structure: 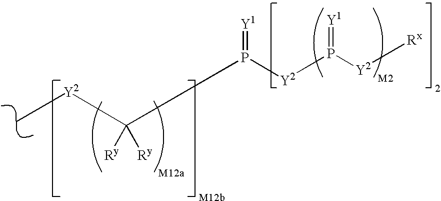
where:
Y¹ is independently O, S, NR^{x}, N(O)(R^{x}), N(OR^{x}), N(O)(O^{x}), or N(N(R^{x})₂);
Y² is independently a bond, O, NR^{x}, N(O)(R^{x}), N(OR^{x}), N(O)(OR^{x}), N(N(R^{x})₂), —S(O)— (sulfoxide), —S(O)₂— (sulfone), —S-(sulfide), or —S—S-(disulfide);
M2 is 0, 1 or 2;
M12a is 1, 2, 3, 4, 5, 6, 7, 8, 9, 10, 11, or 12;
M12b is 0, 1, 2, 3, 4, 5, 6, 7, 8, 9, 10, 11, or 12;
R^{y} is independently H, C₁-C₁₈ alkyl, C₁-C₁₈ substituted alkyl, CC₁₋₂₀ aryl, C₆-C₂₀ substituted aryl, or a protecting group, or where taken together at a carbon atom, two vicinal R^{y} groups form a carbocycle or a heterocycle; and
R^{x} is independently H, C₁-C₁₈ alkyl, C₁-C₁₈ substituted alkyl, C₆-C₂₀ aryl, C₆-C₂₀ substituted aryl, or a protecting group, or the formula: 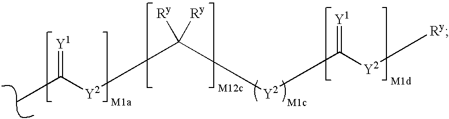
where M1a, M1c, and M1d are independently 0 or 1, and M12c is 0, 1, 2, 3, 4, 5, 6, 7, 8, 9, 10, 11 or 12; and
wherein at least one of R, R¹, R^{2a}, R^{2b}, R³, R⁴, and R⁵ comprises a phosphonate group.

2. A compound according to claim 1 having the structure: 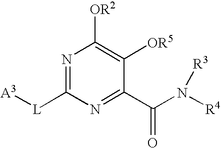
or a pharmaceutically acceptable salt thereof, and including enol and tautomeric resonance isomers.

3. A compound according to claim 1 having the structure: 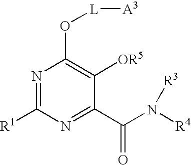
or a pharmaceutically acceptable salt thereof, and including enol and tautomeric resonance isomers.

4. A compound according to claim 1 having the structure: 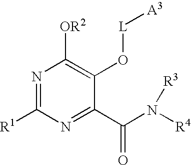
or a pharmaceutically acceptable salt thereof, and including enol and tautomeric resonance isomers.

5. A compound according to claim 1 having the structure: 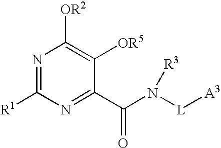
or a pharmaceutically acceptable salt thereof, and including enol and tautomeric resonance isomers.

6. A compound according to claim 1 having the structure: 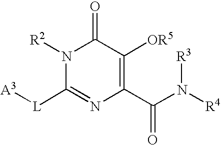
or a pharmaceutically acceptable salt thereof, and including all enol, tautomeric, and resonance isomers, enantiomers, diastereomers, and racemic mixtures thereof.

7. A compound according to claim 1 having the structure: 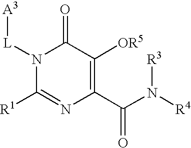
or a pharmaceutically acceptable salt thereof, and including enol and tautomeric resonance isomers.

8. A compound according to claim 1 having the structure: 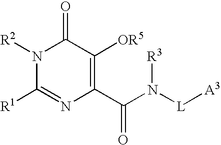
or a pharmaceutically acceptable salt thereof, and including enol and tautomeric resonance isomers.

9. A compound according to claim 1 having the structure: 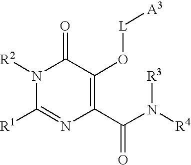
or a pharmaceutically acceptable salt thereof, and including enol and tautomeric resonance isomers.

10. The compound of claim 1 wherein substituted allyl, substituted alkenyl, substituted alkynyl, substituted aryl, and substituted heterocycle are independently substituted with one or more substituents selected from F, Cl, Br, I, OH, amino (—NH₂), ammonium (—NH₃⁺), alkylamino (—NHR), dialkylamino (—NR₂), trialkylammonium (—NR₃⁺), C₁-C₈ alkyl, C₁-C₈ alkylhalide, carboxylate, thiol (—SH), sulfate (—OSO₃R), sulfamate, sulfonate (—SO₃R), 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), allylsulfoxide (—S OR), ester (—C(═O)OR), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ alkyl, C₁-C₈ substituted alkyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, and a prodrug moiety.

11. A compound of claim 1 wherein R^{2a} and R^{2b} are selected from H, C(═O)OR, C(═O)NR₂, C(═O)R, SO₂NR² (sulfamate), and a prodrug moiety.

12. The compound of claim 1 where R³ or R⁴ is 4-fluorobenzyl.

13. The compound of claim 1 wherein at least one of R¹, R^{2a}, R^{2b}, R³, R⁴, and R⁵ comprise a prodrug moiety selected from the structures: 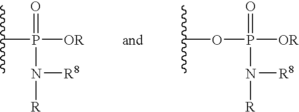
wherein R⁸ is comprised of an ester, an amide, or a carbamate.

14. The compound of claim 1 wherein phosphonate group has the structure:

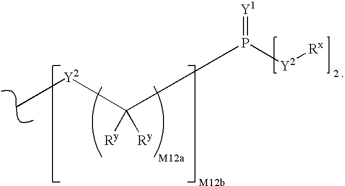

15. The compound of claim 14 wherein phosphonate group has the structure: 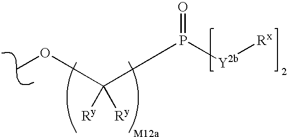
where Y^{2b} is O or N(R^{x}).

16. The compound of claim 14 wherein phosphonate group has the structure: 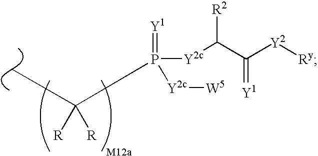
where W⁵ is a carbocycle, and Y^{2c} is O, N(R^{y}) or S.

17. The compound of claim 16 wherein W⁵ is selected from the structures:

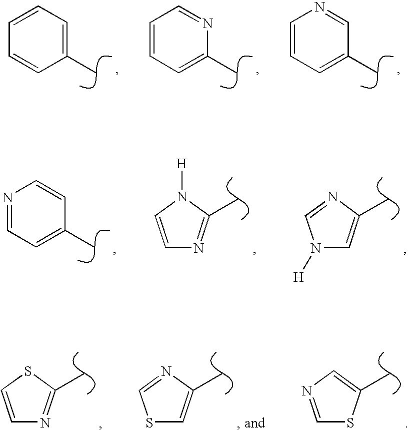

18. The compound of claim 14 wherein phosphonate group has the structure:

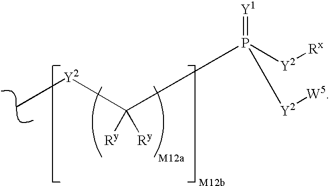

19. The compound of claim 18 wherein phosphonate group has the structure: 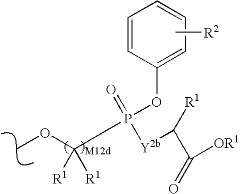
wherein Y^{2b} is O or N(R^{x}); M12d is 1, 2, 3, 4, 5, 6, 7 or 8; R¹ is H or C₁-C₆ alkyl; and the phenyl carbocycle is substituted with 0 to 3 R² groups where R² is C₁-C₆ alkyl or substituted alkyl.

20. The compound of claim 19 wherein phosphonate group has the structure:

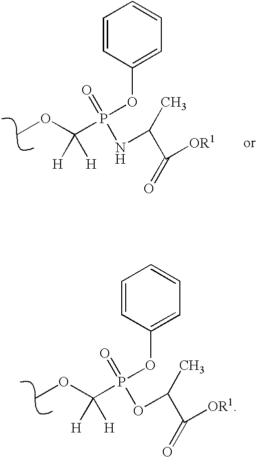

21. The compound of claim 14 wherein R^{x} is selected from the structures:

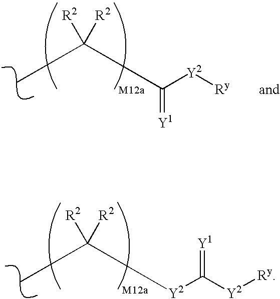

22. The compound of claim 21 wherein R¹ is selected from the structures:

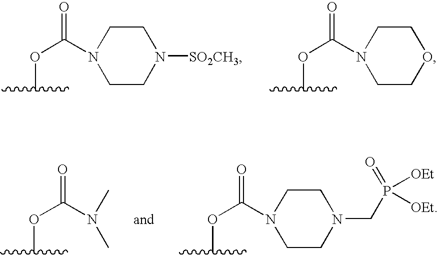

23. The compound of claim 21 wherein R¹ is selected from the structures:

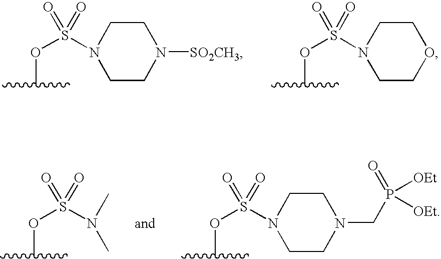

24. A compound of claim 1 wherein R¹ comprises a phosphonate prodrug moiety.

25. The compound of claim 1 wherein R³ or R⁴ is selected from the structures:

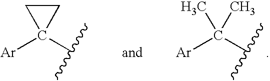

26. The compound of claim 6 wherein L is arylene.

27. The compound of claim 6 wherein L is C₁-C₁₂ alkylene.

28. The compound of claim 26 wherein L is

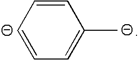

29. The compound of claim 27 wherein L is C₂ alkylene.

30. The compound of claim 6 wherein A³ has the structure:

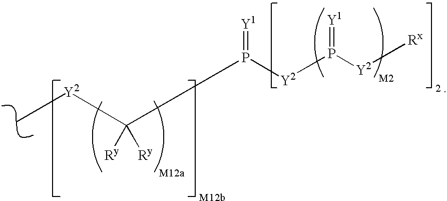

31. The compound of claim 6 wherein A³ has the structure:

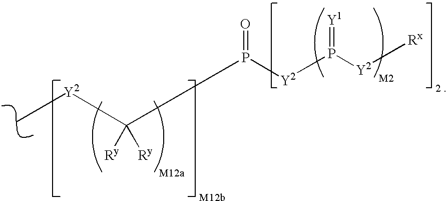

32. The compound of claim 6 wherein A³ has the structure:

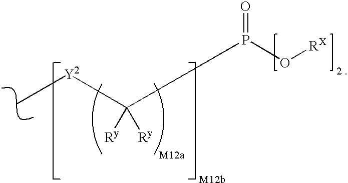

33. The compound of claim 6 wherein A³ has the structure:

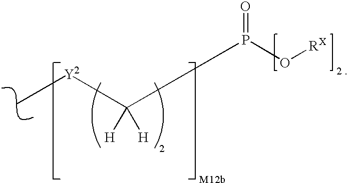

34. The compound of claim 6 wherein A³ has the structure:

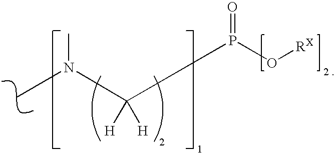

35. The compound of claim 30 wherein A³ has the structure,

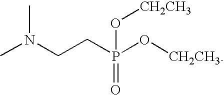

36. The compound of claim 30 wherein A³ has the structure,

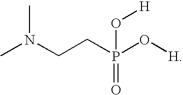

37. A compound of claim 1 having the structure:

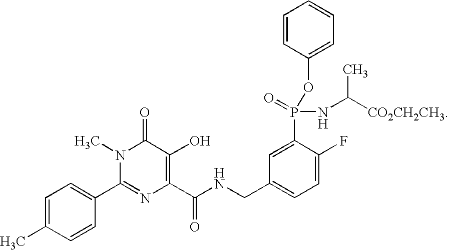

38. A compound of claim 1 having the structure:

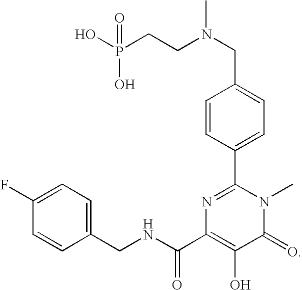

39. A compound of claim 1 having the structure:

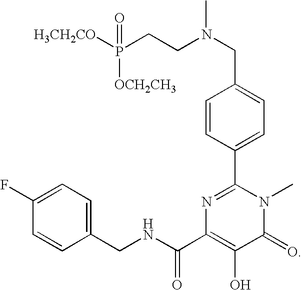

40. A compound of claim 1 having the structure:

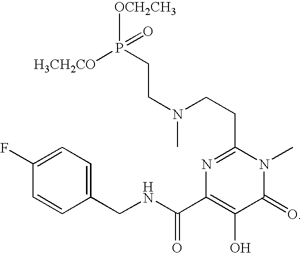

41. A pharmaceutical composition comprising a therapeutically effective amount of a compound of claim 1 and a pharmaceutically acceptable carrier.

42. A pharmaceutical composition comprising a therapeutically effective amount of a compound of claim 1 in combination with a therapeutically effective amount of an AIDS treatment agent selected from:
(1) an AIDS antiviral agent,
(2) an anti-infective agent, and
(3) an immunomodulator.

43. The composition of claim 42 wherein the antiviral agent is an HIV protease inhibitor.

44. A process for making a pharmaceutical composition comprising combining a compound of claim 1 and a pharmaceutically acceptable carrier.

45. A method of inhibiting HIV integrase, comprising the administration to a mammal in need of such treatment of a therapeutically effective amount of a compound of claim 1.

46. A method of treating infection by HIV, or of treating AIDS or ARC, comprising administration to a mammal in need of such treatment of a therapeutically effective amount of a compound of claim 1.
